(19) United States
(12) Patent Application Publication
(10) Pub. No.: US 2014/0286894 A1
(43) Pub. Date: Sep. 25, 2014

(54) COMPOSITIONS COMPRISING CROSSLINKED CATION-BINDING POLYMERS AND CALCIUM AND/OR MAGNESIUM CATIONS, AND USES THEREOF

(71) Applicants: Alan D. Strickland, Lake Jackson, TX (US); George M. Grass, Tahoe City, CA (US)

(72) Inventors: Alan D. Strickland, Lake Jackson, TX (US); George M. Grass, Tahoe City, CA (US)

(73) Assignee: Sorbent Therapeutics, Inc., Sunnyvale, CA (US)

(21) Appl. No.: 13/978,872

(22) PCT Filed: Jan. 10, 2012
(86) PCT No.: PCT/US12/20849

§ 371 Date: Apr. 28, 2014

Related U.S. Application Data
(60) Provisional application No. 61/431,428, filed on Jan. 10, 2011.

(51) Int. Cl. A61K 31/78

(52) U.S. Cl. CPC A61K 31/78
USPC 424/78.31; 526/318.43; 526/318.42

(57) ABSTRACT

The present disclosure relates generally to crosslinked cation-binding polymers comprising monomers containing carboxylic acid groups, wherein the polymer contains calcium and/or magnesium cations that are counterions to about 15% to about 35% of the carboxylic acid groups in the polymer. The present disclosure also relates to methods of preparation of the polymers, and compositions, formulations, and dosage forms containing the polymers, and methods of using the polymers, compositions, formulations, and/or dosage forms to treat various diseases or disorders, including those involving ion and/or fluid imbalances.

### CROSS-REFERENCE TO RELATED APPLICATIONS

This application claims the benefit of U.S. Provisional Application No. 61/431,428, filed on Jan. 10, 2011, which is incorporated by reference herein in its entirety.

### FIELD OF THE DISCLOSURE

The present disclosure relates generally to crosslinked cation-binding polymers comprising monomers comprising carboxylate groups and calcium and/or magnesium cations, wherein the calcium and/or magnesium cations are counterions to about 15% to about 35% of the carboxylate groups in the polymer, and wherein the polymer comprises no more than about 5% sodium cations as counterions to the carboxylate groups in the polymer. The present disclosure also relates to methods of preparation of the polymers or compositions, formulations, and/or dosage forms containing the polymers, and methods of using the polymers or compositions, formulations, and/or dosage forms containing the polymers to treat various diseases or disorders, including those involving ion and/or fluid imbalances.

### BACKGROUND

Numerous diseases and disorders are associated with ion imbalances (e.g., hyperkalemia, hypernatremia, hypercalcemia, and hypermagnesia) and/or increased retention of fluid (e.g., heart failure and end stage renal disease (ESRD)). For example, patients afflicted with an increased level of potassium (e.g., hyperkalemia) may exhibit a variety of symptoms ranging from malaise, palpitations, muscle weakness and, in severe cases, cardiac arrhythmias. Patients afflicted with increased levels of sodium (e.g., hypernatremia) may exhibit a variety of symptoms including, lethargy, weakness, irritability, edema and in severe cases, seizures and coma. Patients afflicted with retention of fluid often suffer from edema (e.g., pulmonary edema, peripheral edema, edema of the legs, etc.) and the buildup of waste products in the blood (e.g., urea, creatinine, other nitrogenous waste products, and electrolytes or minerals such as sodium, phosphate and potassium).

Treatments for diseases or disorders associated with ion imbalances and/or an increased retention of fluid attempt to restore the ion balance and decrease the retention of fluid. For example, treatment of diseases or disorders associated with ion imbalances may employ the use of ion exchange resins to restore ion balance. Treatment of diseases or disorders associated with an increased retention of fluid may involve the use of diuretics (e.g., administration of diuretic agents and/or dialysis, such as hemodialysis or peritoneal dialysis and remediation of waste products that accumulate in the body). Additionally or alternatively, treatment for ion imbalances and/or increased retention of fluid may include restrictions on dietary consumption of electrolytes and water. However, the effectiveness and/or patient compliance with present treatments is less than desired.

### BRIEF SUMMARY OF THE DISCLOSURE

The present disclosure relates generally to crosslinked cation-binding polymers comprising monomers containing carboxylate groups, wherein the polymers further comprise calcium and/or magnesium cations (e.g., calcium cations or magnesium cations or a mixture thereof), wherein the calcium and/or magnesium cations are counterions to about 15% to about 35% of the carboxylate groups in the polymer (alternatively, the polymers comprise calcium and/or magnesium cations that are counterions to about 15% to about 30%, about 20% to about 30%, or about 25% to about 35%, for example, about 25%, of the carboxylate groups in the polymer), and wherein sodium cations are counterions to no more than about 5% (alternatively, no more than about 4%, about 3%, about 2%, or about 1%, about 0.5%, about 0.05%, or about 0.01%) of the carboxylate groups on the polymer.

In some embodiments, at least a portion of the polymer is derived from acrylic acid monomers or acrylic acid derivative monomers. In some embodiments, all or substantially all of the polymer is derived from acrylic acid monomers or acrylic acid derivative monomers. In some embodiments, the polymer is crosslinked polyacrylate. In some embodiments, the polymer comprises calcium cations. The present disclosure also relates to methods of preparation of crosslinked cation-binding polymers comprising monomers that comprise carboxylate groups, for example, crosslinked polyacrylic acid, wherein the polymers further comprise calcium and/or magnesium cations, wherein the calcium and/or magnesium cations are counterions to about 15% to about 35% of the carboxylate groups in the polymer (alternatively, the polymers comprise calcium and/or magnesium cations that are counterions to about 15% to about 30%, about 20% to about 30%, or about 25% to about 35%, for example, about 25%, of the carboxylate groups in the polymer), and wherein the polymers may comprise sodium cations that are counterions to no more than about 5% (alternatively, no more than about 4%, about 3%, about 2%, or about 1%, about 0.5%, about 0.1%, or about 0.05%) of the carboxylate groups in the polymer. Any suitable carboxylic acid-containing monomer known in the art may be used to prepare the compositions as disclosed herein, such as acrylic acid or a derivative thereof. Acrylic acid is a preferred monomer.

The present disclosure also relates to compositions, formulations, and/or dosage forms comprising crosslinked cation-binding polymers comprising monomers that comprise carboxylate groups, for example, crosslinked polyacrylic acid, wherein the polymers further comprise and/or magnesium cations, wherein the calcium and/or magnesium cations are counterions to about 15% to about 35% of the carboxylate groups in the polymer (alternatively, the polymers comprise calcium and/or magnesium cations that are counterions to about 15% to about 30%, about 20% to about 30%, or about 25% to about 35%, for example, about 25%, of the carboxylate groups in the polymer), and wherein the polymer may optionally comprise sodium cations that are counterions to less than about 5%, less than about 4%, less than about 3%, less than about 2%, or less than about 1% of the carboxylate groups. In some embodiments, compositions, formulations, and/or dosage forms according to the present disclosure may optionally further comprise an added base (for example, calcium carbonate). The added base may be included in an amount to provide up to about 0.8 equivalents of base per equivalent of carboxylate groups in the polymer. In alternate embodiments, composition, formulations, and/or dosage forms according to the present disclosure do not comprise an added base.

Any suitable base or combination of two or more bases may be used to prepare compositions as disclosed herein that contain a disclosed polymer and added base. In some embodiments, the composition comprises a base such as an alkali earth metal carbonate, an alkali earth metal acetate, an alkali earth metal oxide, an alkali earth metal bicarbonate, an alkali earth metal hydroxide, an organic base, or combinations thereof. In some embodiments, the base is a calcium base such as calcium carbonate, calcium acetate, calcium oxide, or combinations thereof. In some embodiments, the base is a magnesium base such as magnesium oxide. In some embodiments, the base is an organic base such as lysine, choline, histidine, arginine, or combinations thereof.

The present disclosure also relates to methods of preparation of the polymers and compositions, formulations, and/or dosage forms containing the polymers.

The present disclosure also relates to dosage forms (e.g., oral dosage forms) comprising one or more of the polymers, compositions and/or formulations disclosed herein.

The present disclosure also relates to methods of using the disclosed polymers, compositions, formulations, and/or dosage forms to treat various diseases or disorders, including those involving ion imbalances and/or fluid imbalances (e.g., overloads). In some embodiments, the disease is heart failure. In some embodiments, the disease is heart failure with chronic kidney disease. In some embodiments, the disease is end stage renal disease. In some embodiments, the disease is end stage renal disease with heart failure. In some embodiments, the disease is chronic kidney disease. In some embodiments, the disease is hypertension. In some embodiments, the disease is salt-sensitive hypertension. In some embodiments, the disease is refractory hypertension. In some embodiments, the disease involves an ion imbalance such as hyperkalemia, hypernatremia, hypercalcemia, etc. In some embodiments, the disease or disorder involves a fluid maldistribution or fluid overload state such as edema or ascites.

In some embodiments, the disease or disorder is the result of, or is associated with, administration of another agent (e.g., drug). For example, compositions, formulations, and/or dosage forms according to the present disclosure are useful in treating an increase in a subject's potassium level when co-administered with an agent (e.g., drug) known to cause increases in potassium levels, such as an alpha-adrenergic agonist, a RAAS inhibitor, an ACE inhibitor, an angiotensin II receptor blocker, a beta blocker, an aldosterone antagonist, etc.

These and other embodiments will be described more fully by the detailed description and examples that follow.

### DETAILED DESCRIPTION

The present disclosure relates generally to crosslinked cation-binding polymers comprising monomers that comprise carboxylate groups, e.g., crosslinked polyacrylic acid, and compositions, formulations, and/or dosage forms that contain the polymers, wherein the polymers further comprise calcium and/or magnesium cations (i.e., calcium cations, magnesium cations, or a mixture of calcium and magnesium cations), wherein the calcium and/or magnesium cations are counterions to about 15% to about 35% of the carboxylate groups in the polymer (i.e., the polymer comprises an amount of calcium cations, an amount of magnesium cations, or an amount of a mixture of calcium and magnesium cations sufficient to provide calcium and/or magnesium counterions to about 15% to about 35% of the carboxylate groups in the polymer). Alternatively, the polymer comprises calcium and/or magnesium cations that are counterions to about 15% to about 30%, about 20% to about 30%, about 25% to about 35%, about 15% to about 20%, about 20% to about 25%, or about 25% to about 30% of the carboxylate groups in the polymer. In some embodiments, the polymer comprises calcium and/or magnesium cations that are counterions to about 15%, about 20%, about 25%, about 30%, or about 35% of the carboxylate groups in the polymer. In some embodiments, the calcium and/or magnesium counterions consist of calcium cations. In other embodiments, the calcium and/or magnesium counterions consist of magnesium cations. In further embodiments, the calcium and/or magnesium cations consist of a mixture of calcium and magnesium cations. In some embodiments, the polymers may comprise sodium cations that are counterions to up to about 5% of the carboxylate groups on the polymer. In some embodiments, the polymer comprises sodium cations that are counterions to about 5% of the carboxylate groups on the polymer. In some embodiments, the polymer comprises sodium cations that are counterions to about 4% of the carboxylate groups on the polymer. In some embodiments, the polymer comprises sodium cations that are counterions to about 3% of the carboxylate groups on the polymer. In some embodiments, the polymer comprises sodium cations that are counterions to about 2% of the carboxylate groups on the polymer. In some embodiments, the polymer comprises sodium cations that are counterions to about 1% of the carboxylate groups on the polymer. In some embodiments, the polymer comprises sodium cations that are counterions to less than 1% of the carboxylate groups on the polymer. In some embodiments, the polymer comprises calcium and/or magnesium cations as counterions to about 15% to about 35% of the carboxylate groups on the polymer, sodium cations as counterions up to about 5% of the carboxylate groups on the polymer, and hydrogen cations (e.g., protons) as counterions to about 60% to about 90% of the carboxylate groups on the polymer. In some embodiments, the polymer comprises calcium and/or magnesium cations as counterions to about 15% to about 35% of the carboxylate groups on the polymer and hydrogen cations (e.g., protons) are counterions to the remainder or substantially the remainder of the carboxylate groups on the polymer (e.g., counterions that are not calcium, magnesium, or sodium are hydrogen). It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, crosslinked cation-binding polymers are provided that comprise monomers that comprise carboxylate groups, e.g., crosslinked polyacrylic acid, and compositions, formulations, and/or dosage forms that contain the polymers, wherein the polymers further comprise calcium cations, wherein the calcium cations are counterions to about 15% to about 35% of the carboxylate groups in the polymer (i.e., the polymer comprises an amount of calcium cations sufficient to provide calcium and/or magnesium counterions to about 15% to about 35% of the carboxylate groups in the polymer). Alternatively, the polymer comprises calcium cations that are counterions to about 15% to about 30%, about 20% to about 30%, or about 25% to about 35%. About 15% to about 20%, about 20% to about 25%, or about 25% to about 30% of the carboxylate groups in the polymer. In some embodiments, the polymer comprises calcium cations that are counterions to about 15%, about 20%, about 25%, about 30%, or about 35% of the carboxylate groups in the polymer. In some embodiments, the calcium counterions consist of calcium cations. In some embodiments, the polymers may comprise sodium cations that are counterions to up to about 5% of the carboxylate groups on the polymer. In some embodiments, the polymer comprises sodium cations that are counterions to about 5% of the carboxylate groups on the polymer. In some embodiments, the polymer comprises sodium cations that are counterions to about 4% of the carboxylate groups on the polymer. In some embodiments, the polymer comprises sodium cations that are counterions to about 3% of the carboxylate groups on the polymer. In some embodiments, the polymer comprises sodium cations that are counterions to about 2% of the carboxylate groups on the polymer. In some embodiments, the polymer comprises sodium cations that are counterions to about 1% of the carboxylate groups on the polymer. In some embodiments, the polymer comprises sodium cations that are counterions to less than 1% of the carboxylate groups on the polymer. In some embodiments, the polymer comprises calcium cations as counterions to about 15% to about 35% of the carboxylate groups on the polymer, sodium cations as counterions up to about 5% of the carboxylate groups on the polymer, and hydrogen cations (e.g., protons) as counterions to about 60% to about 90% of the carboxylate groups on the polymer. In some embodiments, the polymer comprises calcium cations as counterions to about 15% to about 35% of the carboxylate groups on the polymer and hydrogen cations (e.g., protons) are counterions to the remainder or substantially the remainder of the carboxylate groups on the polymer (e.g., counterions that are not calcium or sodium are hydrogen). It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, crosslinked cation-binding polymers are provided that comprise monomers that comprise carboxylate groups, e.g., crosslinked polyacrylic acid, and compositions, formulations, and/or dosage forms that contain the polymers, wherein the polymers further comprise magnesium cations, wherein the magnesium cations are counterions to about 15% to about 35% of the carboxylate groups in the polymer (i.e., the polymer comprises an amount of magnesium cations sufficient to provide magnesium counterions to about 15% to about 35% of the carboxylate groups in the polymer). Alternatively, the polymer comprises magnesium cations that are counterions to about 15% to about 30%, about 20% to about 30%, about 25% to about 35%, about 15% to about 20%, about 20% to about 25%, or about 25% to about 30% of the carboxylate groups in the polymer. In some embodiments, the polymer comprises magnesium cations that are counterions to about 15%, about 20%, about 25%, about 30%, or about 35% of the carboxylate groups in the polymer. In some embodiments, the magnesium counterions consist of magnesium cations. In some embodiments, the polymers may comprise sodium cations that are counterions to up to about 5% of the carboxylate groups on the polymer. In some embodiments, the polymer comprises sodium cations that are counterions to about 5% of the carboxylate groups on the polymer. In some embodiments, the polymer comprises sodium cations that are counterions to about 4% of the carboxylate groups on the polymer. In some embodiments, the polymer comprises sodium cations that are counterions to about 3% of the carboxylate groups on the polymer. In some embodiments, the polymer comprises sodium cations that are counterions to about 2% of the carboxylate groups on the polymer. In some embodiments, the polymer comprises sodium cations that are counterions to about 1% of the carboxylate groups on the polymer. In some embodiments, the polymer comprises sodium cations that are counterions to less than 1% of the carboxylate groups on the polymer. In some embodiments, the polymer comprises magnesium cations as counterions to about 15% to about 35% of the carboxylate groups on the polymer, sodium cations as counterions up to about 5% of the carboxylate groups on the polymer, and hydrogen cations (e.g., protons) as counterions to about 60% to about 90% of the carboxylate groups on the polymer. In some embodiments, the polymer comprises magnesium cations as counterions to about 15% to about 35% of the carboxylate groups on the polymer and hydrogen cations (e.g., protons) are counterions to the remainder or substantially the remainder of the carboxylate groups on the polymer (e.g., counterions that are not magnesium or sodium are hydrogen). It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, the polymer comprises calcium and/or magnesium cations as counterions to about 15% to about 35% of the carboxylate groups on the polymer, wherein counterions that are not calcium or magnesium are hydrogen (e.g., counterions that are not calcium or magnesium are hydrogen). In some embodiments, the polymer comprises calcium and/or magnesium cations as counterions to about 15% to about 35% of the carboxylate groups on the polymer and hydrogen cations (e.g., protons) are counterions to the remainder or substantially the remainder of the carboxylate groups on the polymer. It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, the polymer comprises calcium cations as counterions to about 15% to about 35% of the carboxylate groups on the polymer, wherein counterions that are not calcium are hydrogen (e.g., counterions that are not calcium are hydrogen). In some embodiments, the polymer comprises calcium cations as counterions to about 15% to about 35% of the carboxylate groups on the polymer and hydrogen cations (e.g., protons) are counterions to the remainder or substantially the remainder of the carboxylate groups on the polymer. It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, the polymer comprises magnesium cations as counterions to about 15% to about 35% of the carboxylate groups on the polymer, wherein counterions that are not magnesium are hydrogen (e.g., counterions that are not magnesium are hydrogen). In some embodiments, the polymer comprises magnesium cations as counterions to about 15% to about 35% of the carboxylate groups on the polymer and hydrogen cations (e.g., protons) are counterions to the remainder or substantially the remainder of the carboxylate groups on the polymer. It is understood by those of skill in the art that these hydrogen cations are essentially hydrogen and may include small amounts (e.g., less than about 10,000 ppm) of non-hydrogen elements, such as iron, copper, aluminum, arsenic, mercury, manganese, phosphorous, lead, selenium, titanium, and/or zinc.

In some embodiments, the polymer comprises calcium cations as counterions to about 15% to about 35% of the carboxylate groups on the polymer, and sodium cations as counterions to no more than about 5% of the carboxylate groups on the polymer. In such embodiments, the polymer further comprises hydrogen cations (e.g., protons) as counterions to all or substantially all of the carboxylate groups to which calcium and sodium are not counterions (e.g., “free carboxylates”), for example about 95% of the free carboxylates, about 96% of the free carboxylates, about 97% of the free carboxylates, about 98% of the free carboxylates, about 99% of the free carboxylates, about 99.5% of the free carboxylates, or about 100% of the free carboxylates. In some embodiments, the polymer comprises calcium cations as counterions to about 25% of the carboxylate groups on the polymer, sodium cations as counterions to no more than about 5% of the carboxylate groups on the polymer, and hydrogen cations (e.g., protons) as counterions to all or substantially all of the free carboxylates.

In some embodiments, the polymer comprises magnesium cations as counterions to about 15% to about 35% of the carboxylate groups on the polymer and sodium cations as counterions to no more than about 5% of the carboxylate groups on the polymer, and further comprises hydrogen cations (e.g., protons) as counterions to all or substantially all of the carboxylate groups to which magnesium or sodium are not counterions (e.g., “free carboxylates”), for example about 95% of the free carboxylates, about 96% of the free carboxylates, about 97% of the free carboxylates, about 98% of the free carboxylates, about 99% of the free carboxylates, about 99.5% of the free carboxylates, or about 100% of the free carboxylates. In some embodiments, the polymer comprises calcium cations as counterions to about 25% of the carboxylate groups on the polymer, sodium cations as counterions to no more than about 5% of the carboxylate groups on the polymer, and hydrogen cations (e.g., protons) as counterions to all or substantially all of the free carboxylates.

The cross-linked polymer, e.g., cross-linked polyacrylate polymer, comprising calcium and/or magnesium counterions as disclosed herein may absorb at least about 20-fold, 30-fold, or 40-fold or more of its mass in fluid, such as a sodium solution (e.g., a solution of sodium salts, such as a saline solution or a physiological saline solution, for example, 0.154 molar total sodium concentration). For example, saline holding capacity for a disclosed cross-linked cation-binding polymer may be determined in a buffered saline solution, e.g., a buffered saline solution that maintains pH at about 7.

Polymers as described herein and compositions that contain the polymers have unexpected cation binding and/or removal, and/or fluid binding and/or removal properties when administered to an individual (e.g., a mammal, such as a human) and therefore are useful for the treatment of a variety of diseases or disorders, including those involving ion and/or fluid imbalances (e.g., overloads). Surprisingly, ranges of calcium ions have been discovered and are disclosed herein that are optimized for maintaining the cation binding and/or removal properties of the polymer (e.g., for potassium and/or sodium) and/or the fluid binding and/or removal properties of the polymer in individuals, for example, humans. In some embodiments, inclusion of the calcium and/or magnesium ions on the polymer minimizes or prevents acidosis effects from administration of the polymer. In some embodiments, a neutral or substantially neutral acid/base status is maintained in the body of a subject, for example, a human subject. In some embodiments, an acid/base balance associated with a subject does not change, for example, as measured by total serum CO₂, arterial blood pH, urine pH, and/or urine phosphorous, after administration of the polymer, composition comprising a disclosed polymer, formulation comprising a disclosed polymer, and/or dosage form comprising a disclosed polymer as described herein. The present disclosure also relates to methods of preparation of such polymers, and compositions, formulations, and dosage forms containing the polymers. The present disclosure also relates to methods of using such polymers and/or compositions, for example, in dosage forms, for the treatment of various diseases or disorders as disclosed herein, including, for example, heart failure (e.g., with or without chronic kidney disease), end stage renal disease (e.g., with or without heart failure), chronic kidney disease, hypertension (including, e.g., salt sensitive and refractory), hyperkalemia (e.g., any origin), hypernatremia (e.g., any origin), and/or fluid overload states (e.g., edema or ascities).

In some embodiments, compositions, formulations, and/or dosage forms are provided that comprise a base (for example, a calcium-containing base, such as calcium carbonate) and a cross-linked cation-binding polymer comprising monomers that comprise carboxylate groups, such as a cross-linked polyacrylate polymer, wherein the polymer further comprises calcium and/or magnesium cations that are counterions to about 15% to about 35% of the carboxylate groups in the polymer (alternatively, calcium and/or magnesium cations that are counterions to about 15% to about 30%, about 20% to about 30%, or about 25% to about 35% of the carboxylate groups). In some embodiments, the polymer comprises calcium and/or magnesium cations that are counterions to about 15%, about 20%, about 25%, about 30%, or about 35% of the carboxylate groups.

Non-limiting examples of suitable carboxylic acid-containing monomers for production of a polymer as described herein include, for example: acrylic acid and its salts, methacrylic acid and its salts, crotonic acid and its salts, tiglinic acid and its salts, 2-methyl-2-butenoic acid (Z) and its salts, 3-butenoic acid (vinylacetic acid) and its salts, 1-cyclopentene carboxylic acid and its salts, 2-cyclopentene carboxylic acid and its salts; and unsaturated dicarboxylic acids and their salts, such as maleic acid, fumaric acid, itaconic acid, glutaconic acid, and their salts. Polymers may include copolymers of the above monomers. Other cross-linked cation-binding polyelectrolyte polymers may be based on sulfonic acids and their salts, phosphonic acids and their salts, or amines and their salts, for example, acrylic acid with sulfonic acids or salts thereof, phosphonic acids or salts thereof, or amines and their salts. Regardless of the choice of monomer, polymers useful in the present disclosure contain a plurality of carboxylic acid (—C(O)OH) and/or carboxylate (—C(O)O⁻) groups. Polymers of the present disclosure are crosslinked. Any crosslinker known in the art may be used. Crosslinking agents contemplated for use in the present disclosure, include, for example, diethelyeneglycol diacrylate (diacryl glycerol), triallylamine, tetraallyloxyethane, allylmethacrylate, 1,1,1-trimethylolpropane triacrylate (TMPTA), and divinylbenzene. The amount of crosslinking agent used may vary depending on the absorbent characteristics desired. For example, increasing amounts of crosslinking agent will yield polymers with increasing degrees of crosslinking. Such polymers with higher degrees of crosslinking may be preferred over less crosslinked polymers when fluid absorption is unnecessary. For polymers of the present disclosure, an amount of crosslinking may be chosen that yields a polymer with a desired in vitro saline absorption capacity, for example, a saline absorption of at least or greater than about 20, 30, or 40 times its own weight. In some embodiments, the amount of crosslinker used to crosslink polymers according to the present disclosure may range from about 0.08 mol % to about 0.2 mol %.

In certain exemplary embodiments, the crosslinked cation-binding polymer, as described, for example, for inclusion in compositions, formulations, and/or dosage forms and/or for use in methods for treatment of various diseases or disorders as described herein, and/or for use in methods for cation binding and/or removal, and/or fluid binding and/or removal, as described herein, is a crosslinked polyacrylate polymer (i.e., derived from acrylic acid monomers or a salt thereof) that comprises calcium and/or magnesium cations, wherein the calcium and/or magnesium cations are counterions to about 15% to about 35% of the carboxylate groups in the polymer, and wherein sodium cations (if present) are counterions to no more than about 5% of the carboxylate groups in the polymer. For example, the polymer may be a polyacrylate polymer crosslinked with about 0.08 mol % to about 0.2 mol % crosslinker, and for example, may comprise an in vitro saline absorption capacity of at least about 20 times its weight (e.g., at least about 20 grams of saline per gram of polymer, or “g/g”), at least about at least about 30 times its weight, at least about 40 times its weight, at least about 50 times its weight, at least about 60 times its weight, at least about 70 times its weight, at least about 80 times its weight, at least about 90 times its weight, at least about 100 times its weight, or more. In some embodiments, the crosslinked polyacrylate polymer comprises individual particles or particles that are agglomerated (for example, flocculated) to form a larger particle, wherein the individual or agglomerated particle diameter is about 1 to about 10,000 microns (alternatively, about 1 micron to about 10 microns, about 1 micron to about 50 microns, about 10 microns to about 50 microns, about 10 microns to about 200 microns, about 50 microns to about 100 microns, about 50 microns to about 200 microns, about 50 microns to about 1000 microns, about 500 microns to about 1000 microns, about 1000 to about 5000 microns, or about 5000 microns to about 10,000 microns. In one embodiment, the polyacrylate polymer is in the form of small particles that flocculate to form agglomerated particles with a diameter of about 1 micron to about 10 microns.

In some embodiments, administration of such a crosslinked polyacrylate polymer, comprising comprises calcium and/or magnesium cations, wherein the calcium and/or magnesium cations are counterions to about 15% to about 35% of the carboxylate groups in the polymer, does not change or does not significantly change acid/base balance in an individual to whom it is administered, for example, as measured by serum total bicarbonate, arterial blood pH, urine pH, and/or urine phosphorous. In some embodiments, a crosslinked polyacrylate polymer, comprising comprises calcium and/or magnesium cations, wherein the calcium and/or magnesium cations are counterions to about 15% to about 35% of the carboxylate groups in the polymer, is administered with an added base (e.g., up to about 0.8 equivalents of added base per equivalents of carboxylate groups in the polymer), and such administration of the polymer and base does not change or does not significantly change acid/base balance in an individual to whom it is administered, for example, as measured by serum total bicarbonate, arterial blood pH, urine pH, and/or urine phosphorous. In some embodiments, such a crosslinked polyacrylate polymer, comprising calcium and/or magnesium cations, wherein the calcium and/or magnesium cations are counterions to about 15% to about 35% of the carboxylate groups in the polymer, may be administered, optionally with added base as described herein, to an individual for removal of fluid and/or ions, for example, sodium and/or potassium cations, wherein such administration does not change or does not significantly change acid/base balance in the individual, for example, as measured by serum total bicarbonate, arterial blood pH, urine pH, and/or urine phosphorous.

As used herein, the term “cation” or “cations” refers to atomic, polyatomic, or molecular ions having a net positive charge, and may include one such cation or a combination of more than one cation. Non-limiting examples of cations include: hydrogen cations (H⁺), sodium cations (Na⁺), potassium cations (K⁺), magnesium cations (Mg²⁺), calcium cations (Ca²⁺), iron cations (e.g., Fe²⁺, Fe³⁺), and combinations thereof. As used herein, the term “non-hydrogen cation” or “non-hydrogen cations” refers to cation(s) (e.g., as defined above) other than hydrogen (H⁺; proton). Mixtures of more than one cation are within the scope of the terms cation or cations, as used herein. Counterions to carboxylate groups on the polymers described herein are cations. Crosslinked cation-binding polymers as disclosed herein can be described by the percentage of carboxylate groups for which one or more cation serves as a counterion. For example, a polymer according to the present disclosure may be referred to as “25% calcium” to indicate that calcium cations are counterions to about 25% of the carboxylate groups in the polymer. Or, when expressed as a molar ratio, a “25% calcium” polymer according to the present disclosure includes about 12.5 moles of calcium cations (i.e., divalent Ca²⁺ cations) per 100 moles of carboxylate groups in the polymer (e.g., a mole fraction with respect to calcium of 0.125). In another example, a “15% magnesium” polymer according to the present disclosure indicates that magnesium cations (i.e., divalent Mg²⁺ cations) are counterions to about 15% of the carboxylate groups in the polymer (e.g., a mole fraction with respect to magnesium of 0.05). A “15% calcium/15% magnesium” polymer according to the present disclosure likewise indicates that calcium cations are counterions to about 15% of the carboxylate groups in the polymer and magnesium cations are counterions to about 15% of the carboxylate groups in the polymer (e.g., mole fractions of 0.075 for calcium and 0.05 for magnesium). In some embodiments, hydrogen cations (e.g., protons) may be counterions to all or substantially all of the carboxylate groups for which calcium and/or magnesium are not counterions.

In some embodiments, crosslinked cation-binding polymers according to the present disclosure comprise calcium and/or magnesium cations that are counterions to about 15% to about 35% of the carboxylate groups in the polymer, and further comprise one or more additional cations. In some embodiments, the one or more additional cations are monovalent cations such as sodium, potassium, ammonium, arginine, lysine, choline, histidine, serine, and the like. In some embodiments, the one or more additional cations are divalent cations such as iron(II), zinc, a lanthanide, and the like. In some embodiments, the one or more cations are trivalent cations such as aluminum, iron(III), and the like. Nomenclature of a polymer which comprises one or more additional cations thus depends on the identity of the one or more additional cations, the amount of each of the one or more additional cations, and the valency of each of the one or more additional cations. For example, a polymer according to the present disclosure denoted as “25% calcium/9% iron (III)” would indicate that calcium cations are counterions to about 25% of the carboxylate groups in the polymer, trivalent iron cations (Fe³⁺ cations) are counterions to about 9% of the carboxylate groups in the polymer, and hydrogen cations (e.g., protons) are counterions to about 66% of the carboxylate groups in the polymer. To avoid any doubt, a “25% calcium/9% trivalent iron” polymer according to the present disclosure comprises about 12.5 moles of calcium cations and about 3 moles of trivalent iron cations per 100 moles of carboxylate groups in the polymer (e.g., 0.25 equivalents of calcium cations and 0.9 equivalents of iron).

Determination of the percentage of cations that serve as counterions to carboxylate groups in a polymer as disclosed herein can be accomplished by any suitable means known in the art. When the polymer comprises calcium as a counterion to the carboxylate groups of the polymer, the polymer may be referred to as Ca—CLP. When the comprises magnesium as a counterion to the carboxylate groups of the polymer, the polymer may be referred to as Mg—CLP. When the comprises sodium as a counterion to the carboxylate groups of the polymer, the polymer may be referred to as Na—CLP. For example and without limitation, the polymer may be analyzed with an inductively coupled plasma (“ICP”) spectrometer (e.g., by mass spectroscopy (ICP-MS), atomic emission spectroscopy (ICP-AES), or optical emission spectroscopy (ICP-OES)) using methods known to those skilled in the art. The percentage of cations serving as counterions to carboxylate groups in the polymer (e.g., calcium and/or magnesium counterions to the carboxylate groups in the polymer) may be confirmed, for example, by ICP spectroscopy, atomic absorption spectroscopy, ion chromatography, or similar analytic methods. Such methods are well known in the art.

Cation content of polymers disclosed herein may be determined by ICP, including ICP-AES, ICP-MS, or ICP-OES (see, e.g., Example 6). In some exemplary embodiments, content of calcium, magnesium, sodium, potassium, and/or iron may be determined. The ICP analysis may be reported in μg cation/g polymer, which may then be converted to weight percent (wt. %). Weight percent may be converted to % of cations that are counterions to the carboxylate groups in the polymer. The % of cations that are counterions to the carboxylate groups in the polymer determined in different measurements may vary by ±20% or less. For example, the determination of 15% to 35% calcium cations as counterions to carboxylate groups in the polymer may vary in different measurements by ICP (e.g., 15%±20% to 35%±20%.)

For example, an ICP analysis of a crosslinked cation-binding polyacrylate polymer as disclosed herein, comprising acrylic acid monomers comprising carboxylate groups and calcium cations, wherein the calcium cations are counterions to about 15% to about 35% of the carboxylate groups in the polymer, and wherein the polymer comprises no more than about 5% sodium cations as counterions to the carboxylate groups in the polymer, reporting calcium content at 4.0-8.9 wt. % calcium in a polymer containing essentially calcium cations and protons represents about 15% to about 35% calcium as counterions to carboxylate groups in the polyacrylate polymer (e.g., about 15% Ca—CLP to about 35% Ca—CLP), as calculated by the following formula for a polyacrylate polymer:

[x]% Ca—CLP=(72.06)(wt. % Ca)/(20.05−(0.19)(wt. % Ca))

In an embodiment, an ICP analysis that reports calcium content at 5.7 wt. % calcium represents a 22% Ca—CLP polymer (e.g., calcium cations are counterions to about 22% of the carboxylate groups in the polymer), as calculated by the following formula for a polyacrylate polymer:

[x]% Ca—CLP=(72.06)(5.7)/(20.05−(0.19)(5.7))=22% Ca—CLP

In another embodiment, an ICP analysis that reports calcium content at 5.6 wt. % represents a 21% Ca—CLP) polymer (e.g., calcium cations are counterions to about 21% of the carboxylate groups in the polymer). In another embodiment, an ICP analysis that reports calcium content at 7.4 wt. % represents a 29% Ca—CLP) polymer (e.g., calcium cations are counterions to about 29% of the carboxylate groups in the polymer). In Examples 13-14, Ca—CLP of 4.0-8.9 wt. % (e.g., corresponding to 15% to 35%, including 21%, 22%, and 29% Ca—CLP, designated as 25% Ca—CLP, was used.

For example, an ICP analysis that reports magnesium content at 2.5-5.6 wt. % magnesium represents about 15% to about 35% magnesium as counterions to carboxylate groups in a polyacrylate polymer (e.g., about 15% Mg—CLP to about 35% Mg—CLP), as calculated by the following formula for a polyacrylate polymer:

[x]% Mg—CLP=(72.06)(wt. % Mg)/(12.15−(0.11(wt. % Mg))

For example, an ICP analysis that reports sodium content at 0.030 wt. % sodium represents about 0.09% sodium cations as counterions to carboxylate groups in the polymer, as calculated by the following formula for a polyacrylate polymer:

[x]% Na—CLP=(72.06)(wt. % Na)/(23.0−(0.23)(wt. % Na))

For 0.03 wt % sodium, the calculation is as follows:

[x]% Na—CLP=(72.06)(0.030)/(23.0−(0.23)(0.030))=0.09% sodium counterions to the carboxylate groups in the polymer.

In an embodiment, an ICP analysis that reports sodium content at 0.031 wt. % sodium represents a polyacrylate polymer having sodium counterions to about 0.10% of the carboxylate groups in the polymer.
In another embodiment, an ICP analysis that reports sodium content at 0.035 wt. % represents a polyacrylate polymer having sodium counterions to about 0.11% sodium of the carboxylate groups in the polymer.

The above equations are useful for determination of percentage cation when a single type of non-hydrogen cation (e.g., calcium) is present as a counterion to carboxylate groups in the polymer. As understood by persons skilled in the art, the above equations are modified when combinations of types cations are present (e.g., calcium, magnesium, and/or sodium).

Compositions, formulations, and/or dosage forms comprising a polymer as disclosed herein may optionally additionally comprise and/or be co-administered with a base (alternatively termed an alkali). As used herein, the term base may refer to a suitable compound or mixture of compounds that is capable of increasing the pH of the blood or other bodily fluids. Exemplary bases include, but are not limited to, calcium carbonate, calcium acetate, magnesium oxide, calcium oxide, potassium citrate, potassium acetate, and sodium bicarbonate. Generally, inorganic and organic bases can be used, provided they are physiologically and/or clinically acceptable. To be acceptable, the dose and route of administration of the specific base are important considerations. For example, oral administration of even small amounts of sodium hydroxide would cause local tissue damage and would not be acceptable on this basis while administration of intermittent, small amounts of sodium hydroxide intravenously is performed routinely. Similarly, though lithium carbonate or rubidium acetate would be an acceptable base, only small amounts could be used due to the effects of the lithium or the rubidium, regardless of the route of administration.

In some embodiments, compositions, formulations, and/or dosage forms comprising a polymer as disclosed herein additionally comprise a base, wherein the base is added during preparation or formulation of the composition, formulation and/or dosage form and is present in an amount sufficient to provide up to about 0.8 equivalents of base per equivalent of carboxylate groups in the polymer (alternatively, the base is present in an amount sufficient to provide about 0.3 to about 0.6 or about 0.35 to about 0.5 equivalents per equivalent of carboxylate groups in the polymer; alternatively, the base is present in an amount sufficient to provide about 0.65 to about 0.75, such as about 0.66, about 0.70, about 0.73, about 0.74 equivalents of base per equivalent of carboxylate groups in the polymer). For example, the composition, formulation, and/or dosage form may contain a disclosed polymer that contains calcium and/or magnesium cations that are counterions to about 15% to about 35% of the carboxylate groups (i.e., calcium and/or magnesium counterions that are added during manufacture of the polymer) and base that is added during preparation or formulation of the composition, formulation, and/or dosage form in an amount sufficient to provide up to about 0.8 equivalents per equivalent of carboxylate groups in the polymer.

In some embodiments, the base is present, for example, in a composition, formulation, and/or dosage form comprising a disclosed polymer and/or is co-administered with a disclosed polymer, in an amount sufficient to provide an equivalents ratio of up to about 85 equivalents of base per equivalent (e.g., mole) of carboxylic acid groups in the polymer. As used herein, the term “equivalents” or “equivalents ratio” (“ER”) refers to the ratio between the number of units (e.g., equivalents) of base present in the composition and the number of units (e.g., moles) of carboxylic acid groups in the polymer. A monobasic base provides one equivalent of base per mole of monobasic base. A dibasic base provides two equivalents of base per mole of dibasic base. A tribasic base provides three equivalents of base per mole of tribasic base. For example, a composition comprising a polymer derived from polymerization and crosslinking of 1.0 mole of acrylic acid monomers may contain up to about 0.8 moles of a monobasic base, such as a bicarbonate. If a dibasic base is used, such as a carbonate, a composition comprising 1.0 mole of carboxylic acid groups may contain up to about 0.425 moles of the dibasic base.

In some embodiments, compositions of the present disclosure comprise a monobasic base present in an amount sufficient to provide from up to about 0.8 moles of base per mole of carboxylic acid groups in the polymer, for example about 0.5 moles of base, about 0.1 moles of base, about 0.15 moles of base, about 0.2 moles of base, about 0.25 moles of base, about 0.3 moles of base, about 0.35 moles of base, about 0.4 moles of base, about 0.45 moles of base, about 0.5 moles of base, about 0.55 moles of base, about 0.6 moles of base, about 0.65 moles of base, about 0.7 moles of base, about 0.75 moles of base, or about 0.8 moles of base per mole of carboxylic acid groups in the polymer. In some embodiments, compositions of the present disclosure comprise a monobasic base present in an amount sufficient to provide from about 0.2 moles of base to about 0.8 moles of base of base, for example about 0.2 moles of base, about 0.25 moles of base, about 0.3 moles of base, about 0.35 moles of base, about 0.4 moles of base, about 0.45 moles of base, 0.5 moles of base, about 0.55 moles of base, about 0.6 moles of base, about 0.65 moles of base, about 0.7 moles of base, about 0.75 moles of base, or about 0.8 moles of base per mole of carboxylate groups in the polymer. In some embodiments, compositions of the present disclosure comprise a monobasic base present in an amount sufficient to provide from about 0.3 moles of base to about 0.6 moles of base of base, for example about 0.3 moles of base, about 0.35 moles of base, about or 0.4 moles of base, about 0.45 moles of base, about 0.5 moles of base, about 0.55 moles of base, or about 0.6 moles of base per mole of carboxylate groups in the polymer. In some embodiments, compositions of the present disclosure comprise a monobasic base present in an amount sufficient to provide about 0.5 moles of base per mole of carboxylate groups in the polymer.

In some embodiments, compositions of the present disclosure comprise a dibasic base present in an amount sufficient to provide from up to about 0.425 moles of base per mole of carboxylic acid groups in the polymer, for example about 0.05 moles of base, about 0.075 moles of base, about 0.1 moles of base, about 0.125 moles of base, about 0.15 moles of base, about 0.175 moles of base, about 0.2 moles of base, about 0.225 moles of base, about 0.25 moles of base, about 0.275 moles of base, about 0.3 moles of base, about 0.325 moles of base, about 0.35 moles of base, about 0.375 moles of base, about 0.4 moles of base, or about 0.425 moles of base per mole of carboxylic acid groups in the polymer. In some embodiments, compositions of the present disclosure comprise a dibasic base present in an amount sufficient to provide from about 0.1 moles of base to about 0.4 moles of base of base, for example about 0.1 moles of base, about 0.125 moles of base, about 0.15 moles of base, about 0.175 moles of base, about 0.2 moles of base, about 0.225 moles of base, about 0.25 moles of base, about 0.275 moles of base, about 0.3 moles of base, about 0.325 moles of base, about 0.35 moles of base, about 0.375 moles of base, or about 0.4 moles of base of base per mole of carboxylate groups in the polymer. In some embodiments, compositions of the present disclosure comprise a dibasic base present in an amount sufficient to provide from about 0.15 moles of base to about 0.25 moles of base of base, for example about 0.15 moles of base, about 0.175 moles of base, about or 0.2 moles of base, about 0.225 moles of base, or about 0.5 moles of base per mole of carboxylate groups in the polymer. In some embodiments, compositions of the present disclosure comprise a dibasic base present in an amount sufficient to provide about 0.25 moles of base per mole of carboxylate groups in the polymer.

In some embodiments, compositions of the present disclosure comprise a tribasic base present in an amount sufficient to provide up to about 0.28 moles of base per mole of carboxylic acid groups in the polymer, for example about 0.017 moles of base, about 0.033 moles of base, about 0.05 moles of base, 0.065 moles of base, about 0.07 moles of base, about 0.075 moles of base, about 0.08 moles of base, about 0.085 moles of base, about 0.09 moles of base, about 0.095 moles of base, about 0.1 moles of base, about 0.105 moles of base, about 0.11 moles of base, about 0.115 moles of base, about 0.12 moles of base, about 0.125 moles of base, about 0.13 moles of base, about 0.135 moles of base, about 0.14 moles of base, about 0.145 moles of base, about 0.15 moles of base, about 0.155 moles of base, about 0.16 moles of base, about 0.165 moles of base, about 0.17 moles of base, about 0.175 moles of base, about 0.18 moles of base, about 0.185 moles of base, about 0.19 moles of base, about 0.195 moles of base, about 0.2 moles of base, about 0.205 moles of base, about 0.21 moles of base, about 0.215 moles of base, about 0.22 moles of base, about 0.225 moles of base, about 0.23 moles of base, about 0.235 moles of base, about 0.24 moles of base, about 0.245 moles of base, about 0.25 moles of base, about 0.255 moles of base, about 0.26 moles of base, about 0.265 moles of base, about 0.27 moles of base, about 0.275 moles of base, or about 0.28 moles of base per mole of carboxylic acid groups in the polymer. In some embodiments, compositions of the present disclosure comprise a tribasic base present in an amount sufficient to provide from about 0.065 moles of base to about 0.26 moles of base of base, for example about 0.065 moles of base, about 0.07 moles of base, about 0.075 moles of base, about 0.08 moles of base, about 0.085 moles of base, about 0.09 moles of base, about 0.095 moles of base, about 0.1 moles of base, about 0.105 moles of base, about 0.11 moles of base, about 0.115 moles of base, about 0.12 moles of base, about 0.125 moles of base, about 0.13 moles of base, about 0.135 moles of base, about 0.14 moles of base, about 0.145 moles of base, about 0.15 moles of base, about 0.155 moles of base, about 0.16 moles of base, about 0.165 moles of base, about 0.17 moles of base, about 0.175 moles of base, about 0.18 moles of base, about 0.185 moles of base, about 0.19 moles of base, about 0.195 moles of base, about 0.2 moles of base, about 0.205 moles of base, about 0.21 moles of base, about 0.215 moles of base, about 0.22 moles of base, about 0.225 moles of base, about 0.23 moles of base, about 0.235 moles of base, about 0.24 moles of base, about 0.245 moles of base, about 0.25 moles of base, about 0.255 moles of base, or about 0.26 moles of base per mole of carboxylate groups in the polymer. In some embodiments, compositions of the present disclosure comprise a tribasic base present in an amount sufficient to provide from about 0.1 moles of base to about 0.2 moles of base of base, for example about 0.1 moles of base, about 0.105 moles of base, about 0.11 moles of base, about 0.115 moles of base, about 0.12 moles of base, about 0.125 moles of base, about 0.13 moles of base, about 0.135 moles of base, about 0.14 moles of base, about 0.145 moles of base, about 0.15 moles of base, about 0.155 moles of base, about 0.16 moles of base, about 0.165 moles of base, about 0.17 moles of base, about 0.175 moles of base, about 0.18 moles of base, about 0.185 moles of base, about 0.19 moles of base, about 0.195 moles of base, or about 0.2 moles of base per mole of carboxylate groups in the polymer. In some embodiments, compositions of the present disclosure comprise a tribasic base present in an amount sufficient to provide about 0.17 moles of base per mole of carboxylate groups in the polymer.

In some embodiments, compositions, formulations, and/or dosage forms of the present disclosure comprise more than one base (e.g., one or more monobasic bases, one or more dibasic bases, one or more tribasic bases, etc.). In such embodiments, the compositions comprise an amount of each base such that the total number of equivalents of base present is up to about 0.8 equivalents per mole of carboxylic acid groups in the polymer, for example, about 0.2 equivalents to about 0.8 equivalents, or about 0.3 equivalents to about 0.6 equivalents, per mole of carboxylic acid groups in the polymer.

Thus, as one example embodiment, a composition according to the present invention that comprises 1.0 mole of carboxylic acid groups and 0.1 moles of sodium bicarbonate may also comprise from about 0.05 moles to about 0.375 moles of a dibasic base such as magnesium carbonate. In such an embodiment, the total equivalents of base would be equal to 0.1+(2) (about 0.05 to about 0.375), or about 0.2 to about 0.8 equivalents of base.

In some embodiments, the base is present in an amount sufficient to provide from up to about 0.8 equivalents of base, for example about 0.05 equivalents, about 0.1 equivalents, about 0.15 equivalents, 0.2 equivalents, about 0.25 equivalents, about 0.3 equivalents, about 0.35 equivalents, about 0.4 equivalents, about 0.45 equivalents, about 0.5 equivalents, about 0.55 equivalents, about 0.6 equivalents, about 0.65 equivalents, about 0.7 equivalents, about 0.75 equivalents, about 0.8 equivalents, about 0.8 equivalents, about 0.9 equivalents, or about 0.95 equivalents of base per equivalent of carboxylic acid groups in the polymer. In some embodiments, the base is present in an amount sufficient to provide from about 0.5 equivalents to about 0.8 equivalents of base, for example about 0.5 equivalents, about 0.55 equivalents, about 0.6 equivalents, about 0.65 equivalents, about 0.7 equivalents, about 0.75 equivalents, about 0.8 equivalents, or about 0.8 equivalents of base per equivalent of carboxylate groups in the polymer. In some embodiments, the base is present in an amount sufficient to provide from about 0.3 equivalents to about 0.6 equivalents of base, for example, about 0.3 equivalents, about 0.35 equivalents, about 0.4 equivalents, about 0.45 equivalents, about 0.5 equivalents, about 0.55 equivalents, or about 0.6 equivalents base per equivalent of carboxylate groups in the polymer. In some embodiments, the base is present in an amount sufficient to provide about 0.5 equivalents of base per equivalent of carboxylate groups in the polymer.

In some embodiments, a composition, formulation, and/or dosage form contains a polymer that comprises calcium and/or magnesium cations that are counterions to about 15% to about 35% of the carboxylate groups (e.g., calcium and/or magnesium counterions that are added during manufacture of the polymer) and added base (e.g., base added during preparation or formulation of the composition, formulation, and/or dosage form that contains the polymer) in an amount sufficient to provide up to about 0.8 equivalents per equivalent of carboxylate groups in the polymer. In one embodiment, the disclosed polymer contains about 25% calcium and/or magnesium (e.g., calcium and/or magnesium counterions added during manufacture of the polymer) and a composition, formulation, and/or dosage form that contains the polymer comprises about 0.5 equivalents of added base. In another embodiment, the disclosed polymer contains about 25% calcium and/or magnesium (e.g., calcium and/or magnesium counterions added during manufacture of the polymer) and a composition, formulation, and/or dosage form that contains the polymer comprises about 0.35 equivalents of added base.

In some embodiments, a composition, formulation, and/or dosage form contains a crosslinked polyacrylate polymer that comprises calcium and/or magnesium cations that are counterions to about 15% to about 35% of the carboxylate groups (e.g., calcium and/or magnesium counterions that are added during manufacture of the polymer) and added base (e.g., base added during preparation or formulation of the composition, formulation, and/or dosage form that contains the polymer) in an amount sufficient to provide up to about 0.8 equivalents per equivalent of carboxylate groups in the polymer. In one embodiment, the crosslinked polyacrylate polymer contains about 25% calcium (e.g., calcium counterions added during manufacture of the polymer) and a composition, formulation, and/or dosage form that contains the polymer comprises about 0.5 equivalents of base. In another embodiment, the crosslinked polyacrylate polymer contains about 25% calcium (e.g., calcium counterions added during manufacture of the polymer) and a composition, formulation, and/or dosage form that contains the polymer comprises about 0.35 equivalents of base.

In some embodiments, a disclosed polymer, e.g., a crosslinked polyacrylate polymer, comprises calcium and/or magnesium cations that are counterions to about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35% of the carboxylate groups in the polymer (i.e., calcium and/or magnesium counterions added during manufacture of the polymer) and a composition, formulation, and/or dosage form that contains the polymer comprises 0.05 equivalents, about 0.1 equivalents, about 0.15 equivalents, about 0.2 equivalents, about 0.25 equivalents, about 0.3 equivalents, about 0.35 equivalents, about 0.4 equivalents, about 0.45 equivalents, about 0.5 equivalents, about 0.6 equivalents, about 0.7 equivalents, about 0.75 equivalents, or about 0.8 equivalents of added base per equivalent of carboxylic acid groups in the polymer (e.g., base added during preparation and/or formulation of the composition, formulation, and/or dosage form that contains the polymer).

In some embodiments, a polymer or composition, formulation, or dosage form containing a polymer as disclosed herein is administered with added base in the same composition, formulation, or dosage form with the polymer or in a separate composition, formulation, or dosage form from the polymer, wherein the base is present in an amount sufficient to provide up to about 0.8 equivalents of base per equivalent of carboxylate groups in the polymer (alternatively, the base is present in an amount sufficient to provide about 0.3 to about 0.6 or about 0.35 to about 0.5 equivalents per equivalent of carboxylate groups in the polymer; alternatively, the base is present in an amount sufficient to provide about 0.65 to about 0.75, such as about 0.66, about 0.70, about 0.73, about 0.74 equivalents of base per equivalent of carboxylate groups in the polymer). For example, a disclosed polymer that contains calcium and/or magnesium counterions to about 25% of the carboxylate groups on the polymer may be administered with base in an amount sufficient to provide about 0.5 equivalents of added base per equivalent of carboxylate groups in the polymer in the same or separate composition, formulation, or dosage form as the polymer. In another example, a disclosed polymer that contains calcium and/or magnesium counterions to about 25% of the carboxylate groups on the polymer may be administered with base in an amount sufficient to provide about 0.35 equivalents of added base per equivalent of carboxylate groups in the polymer.

In some embodiments, the base is one or more of: an alkali metal hydroxide, an alkali metal acetate, an alkali metal carbonate, an alkali metal bicarbonate, an alkali metal oxide, an alkaline earth metal hydroxide, an alkaline earth metal acetate, an alkaline earth metal carbonate, an alkaline earth metal bicarbonate, an alkaline earth metal oxide, and an organic base. In some embodiments, the base is choline, lysine, arginine, histidine, a pharmaceutically acceptable salt thereof, or a combination thereof. In some embodiments, the base is an acetate, a butyrate, a propionate, a lactate, a succinate, a citrate, an isocitrate, a fumarate, a malate, a malonate, an oxaloacetate, a pyruvate, a phosphate, a carbonate, a bicarbonate, a lactate, a benzoate, a sulfate, a lactate, a silicate, an oxide, an oxalate, a hydroxide, an amine, a dihydrogen citrate, or a combination thereof. In some embodiments, the base is a bicarbonate, a carbonate, an oxide, or a hydrochloride. In related embodiments, the base is one or more of: calcium bicarbonate, calcium carbonate, calcium oxide, and calcium hydroxide. In some embodiments, the base is a lithium salt, a sodium salt, a potassium salt, a magnesium salt, a calcium salt, an aluminum salt, a rubidium salt, a barium salt, a chromium salt, a manganese salt, an iron salt, a cobalt salt, a nickel salt, a copper salt, a zinc salt, an ammonium salt, a lanthanum salt, a choline salt, or a serine salt of to any of the foregoing anions or anion combinations.

In some embodiments, the base may be selected to avoid increasing a level of a particular cation associated with the subject. For example, a method of treatment for hyperkalemia in a subject would preferably include administering a base that does not include potassium cations, in conjunction with a polymer or composition, formulation or dosage form containing a polymer as disclosed herein. Similarly, a composition according to the present disclosure intended to treat hypernatremia in a subject would preferably contain a base that does not include sodium cations

In some embodiments, the base may be selected to specifically increase the amount of a particular cation important in the disease or condition of the subject. For example, a method of treatment for hyponatremia, for example, in a subject suffering simultaneously from hyperkalemia and hyponatremia, would preferably contain a base that either includes sodium cations or alters the polymer binding in such a manner that fewer sodium cations are removed by the polymer.

In some embodiments, the disclosed polymers and compositions, formulations, and/or dosage forms containing the polymers described herein have superior manufacturing and processing properties in comparison to analogous polymers, compositions, formulations, and/or dosage forms wherein the carboxylate groups of the polymer are bound to hydrogen cations (e.g., protons; H⁺) instead of calcium cations at the levels described herein. Typically, polymers with predominantly unneutralized carboxylate groups (e.g., more than about 95% of the carboxylates are bound to hydrogen cations) are very adhesive, which may result in manufacturing difficulties and poor oral delivery or mucoadhesive properties. For example, certain methods for preparing the disclosed polymers, compositions, formulations, and/or dosage forms require transferring the polymer from one vessel to another, drying the polymer, grinding or milling to form a powder, filtering the polymer, etc. The adhesive properties associated with polymer having predominantly unneutralized carboxylate groups may render one these exemplary processes difficult, time-consuming, cost-inefficient, or sub-optimal for scale up.

Generally speaking, processing of polycarboxylate polymers, handling of carboxylate polymer, and oral delivery properties of polycarboxylate polymers improve with increasing levels of bound non-hydrogen cations, e.g., calcium ions and/or magnesium ions. For example, polymers disclosed herein have about calcium and/or, magnesium counterions to about 15% to about 35% of the carboxylate groups in the polymer. Such polymers are characterized by dramatically improved manufacturability due to greatly reduced adhesive properties. In addition, the adhesive properties associated with polymers having predominantly unneutralized carboxylate groups lead to poor oral delivery properties as they generally hydrate rapidly when exposed to saliva, becoming bioadhesive. The hydrated form of the polymer causes the material to adhere to oral tissue, including teeth, which can lead to irritation. In contrast, polymers disclosed herein, which have calcium and/or magnesium counterions to about 15% to about 35% of the carboxylate groups in the polymer, possess improved oral delivery properties.

In some embodiments, a polymer and/or composition of the present disclosure has an in vitro saline absorption capacity of greater than or at least about 20 times its own weight (e.g., greater than or at least about 20 grams of saline per gram of composition, or “g/g”). In related embodiments, the polymer and/or composition has an in vitro saline absorption capacity of about 20 times, about 25 times, about 30 times, about 35 times, about 40 times, about 45 times, about 50 times, about 55 times, about 60 times, about 65 times, about 70 times, about 75 times, about 80 times, about 85 times, about 90 times, about 95 times, or about 100 times its own weight, or more. Measurement of the in vitro saline holding capacity of the polymers and compositions according to the present disclosure may be accomplished by any method known in the art, for example, methods as described in Examples 8 and 9.

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 35% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 30% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 20% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 30% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 25% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 30% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 35% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 35% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 30% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 20% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 30% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 25% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 30% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 35% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 35% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 30% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 20% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 30% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 25% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 30% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 35% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 35% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 30% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 20% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 30% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 25% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 30% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 35% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 35% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 30% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 15% to about 20% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 30% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 20% to about 25% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 30% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% to about 35% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium and/or magnesium cations that are counterions to about 25% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 35% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 30% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 20% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 30% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 25% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, crosslinked cation-binding polymer comprising monomers (e.g., acrylic acid) comprising carboxylate groups is a crosslinked polyacrylate, wherein said polymer contains calcium cations that are counterions to about 25% to about 30% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% to about 35% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% of the carboxylate groups of said polymer, and wherein the polymer comprises no more than about 5% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 35% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 30% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 20% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 30% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 25% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% to about 30% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% to about 35% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% of the carboxylate groups of said polymer, and wherein the polymer comprises no more than about 4% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 35% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, crosslinked cation-binding polymer comprising monomers (e.g., acrylic acid) comprising carboxylate groups is a crosslinked polyacrylate, wherein said polymer contains calcium cations that are counterions to about 15% to about 30% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 20% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 30% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 25% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% to about 30% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% to about 35% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% of the carboxylate groups of said polymer, and wherein the polymer comprises no more than about 3% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 35% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 30% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 20% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 30% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 25% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% to about 30% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% to about 35% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% of the carboxylate groups of said polymer, and wherein the polymer comprises no more than about 2% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 35% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 35% (0.35 equivalents), the amount of base is about 0.6 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 30% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 15% to about 20% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 15% (0.15 equivalents), the amount of base is about 0.80 equivalents base and with counterions to about 20% (0.20 equivalents), the amount of base is about 0.75 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 30% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 20% to about 25% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 20% (0.20 equivalents), the amount of base is about 0.75 equivalents base and with counterions to about 25% (0.25 equivalents), the amount of base is about 0.70 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% to about 30% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 30% (0.30 equivalents), the amount of base is about 0.65 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% to about 35% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base and with counterions to about 35% (0.35 equivalents), the amount of base is about 0.60 equivalents base).

In one embodiment, a crosslinked cation-binding polymer comprising monomers (e.g., acrylic acid) comprising carboxylate groups is a crosslinked polyacrylate, wherein said polymer contains calcium cations that are counterions to about 25% of the carboxylate groups of said polymer, and wherein the polymer comprises no more than about 1% sodium cations as counterions to the carboxylate groups of said polymer. In some embodiments, compositions, formulations and/or dosage forms may comprise such a polymer, wherein base is present in an amount up to a total of about 0.95 equivalents of carboxylic acid groups of the polymer (e.g., with counterions to about 25% (0.25 equivalents), the amount of base is about 0.70 equivalents base).

The present disclosure also relates to methods of using the polymers, and compositions, formulations, and/or dosage forms containing the polymers disclosed herein, with or without added base, to treat various diseases and disorders, ion imbalances, and fluid imbalances.

In some embodiments, the disease or disorder is one or more of: heart failure, a renal insufficiency disease, end stage renal disease, liver cirrhosis, chronic renal insufficiency, chronic kidney disease, fluid overload, fluid maldistribution, edema, pulmonary edema, peripheral edema, angioneurotic edema, lymphedema, nephrotic edema, idiopathic edema, ascites, cirrhotic ascites, chronic diarrhea, excessive interdialytic weight gain, high blood pressure, hyperkalemia, hypernatremia, abnormally high total body sodium, hypercalcemia, tumor lysis syndrome, head trauma, an adrenal disease, Addison's disease, salt-wasting congenital adrenal hyperplasia, hyporeninemic hypoaldosteronism, hypertension, salt-sensitive hypertension, refractory hypertension, hyperparathyroidism, renal tubular disease, rhabdomyolysis, electrical burns, thermal burns, crush injuries, renal failure, acute tubular necrosis, insulin insufficiency, hyperkalemic periodic paralysis, hemolysis, malignant hyperthermia, pulmonary edema secondary to cardiogenic pathophysiology, pulmonary edema with non-cardiogenic origin, drowning, acute glomerulonephritis, aspiration inhalation, neurogenic pulmonary edema, allergic pulmonary edema, high altitude sickness, Adult Respiratory Distress Syndrome, traumatic edema, cardiogenic edema, allergic edema, urticarial edema, acute hemorrhagic edema, papilledema, heatstroke edema, facial edema, eyelid edema, angioedema, cerebral edema, scleral edema, nephritis, nephrosis, nephrotic syndrome, glomerulonephritis, renal vein thrombosis, and/or premenstrual syndrome.

In some embodiments, the disease or disorder is the result of, or is associated with, administration of another drug. For example, compositions and/or dosage forms as disclosed herein are useful in treating an increase in a subject's potassium level when co-administered with a drug known to cause increases in potassium levels. In some embodiments, such a drug is an alpha-adrenergic agonist, a RAAS inhibitor, an ACE inhibitor, an angiotensin II receptor blocker, a beta blocker, an aldosterone antagonist, etc.

### Preparation of Crosslinked Cation-Binding Polymers

Crosslinked cation-binding polymers, including, for example, polyelectrolyte polymers, such as polyacrylate polymers, etc., may be prepared by methods known in the art, including by suspension methods and aqueous one-phase methods (e.g., Buchholz, F. L. and Graham, A. T., “Modern Superabsorbent Polymer Technology,” John Wiley & Sons (1998)) and by precipitation polymerization (see, e.g., European Patent Application No. EP0459373A2). Such methods may include manufacture of polyelectrolyte polymers by inverse suspension polymerization. Polymers with differential properties may be prepared that are useful as designer therapeutics for different diseases and disorders, including those involving an ion imbalance and/or a fluid imbalance. For example, methods are provided for washing the cross-linked polymer with an acid to replace bound counterions other than hydrogen with hydrogen. The polymeric material, including for example polymeric beads, may be further processed by milling or grinding the polymeric material into particles. A polymer as described herein may contain many carboxylic acid groups, for example, polyacrylic acid, which may be reacted with alkali metals, e.g., calcium, to produce a polycarboxylate, for example, polyacrylate. Many of these polycarboxylates act as superabsorbent polymers, absorbing over twenty times their mass in vitro in 0.9% saline (0.15 M sodium solution. Exemplary methods are provided below.

1. Manufacture of Crosslinked Cation-Binding Polymers

Cross-linked cation-binding polymers, including cross-linked polyacrylate and/or polyacrylic acid polymers, may be prepared by commonly known methods in the art. In an exemplary method, cross-linked polyelectrolyte polymers may be prepared as a suspension of drops of aqueous solution in a hydrocarbon, for example, a liquid hydrocarbon (e.g., by inverse suspension polymerization).

Cross-linked polyacrylate polymers may be prepared by polymerization of partially neutralized acrylic acid in an aqueous environment where an appropriate cross-linker is present in small quantities. Given that there is an inverse relationship between the amount of fluid the polymer will absorb and the degree of cross-linking of the polymer, it is desirable to have the minimum cross-linking possible to still produce a resin, for example, a resin that is suitable for use in methods as described herein. However, there is also an inverse relationship between the degree of cross-linking and the percentage of polymer chains that do not cross-link. Non-crosslinked polymer is soluble and does not contribute to the absorbency of the resin since it dissolves in the fluid. For example, polyacrylates can be designed to absorb about 35 times their mass in physiological saline as a compromise between maximal absorbency and minimal soluble polymer.

Since the amount of reactants used in an inverse suspension polymerization reaction varies depending upon the size of the reactor, the precise amount of each reactant used in the preparation of cross-linked polyelectrolyte polymer, such as polyacrylate, may be determined by one of skill in the art. For example, in a five-hundred gallon reactor, about 190 to 200 pounds (roughly 85 to 90 kg) of acrylic acid may be used while in a three liter reactor 150 to 180 g of acrylic acid may be used. Accordingly, the amount of each reactant used for the preparation of cross-linked polyacrylate is expressed as a weight ratio to acrylic acid. Thus, acrylic acid weight is taken as 1.0000 and other compounds are represented in relation to this value. Exemplary amounts of reactants used for the preparation of cross-linked polyacrylate by an inverse suspension polymerization are presented in Table 1.

TABLE 1
Exemplary amounts of reactants in an
inverse suspension polymerization

| Substance | Low value | High Value |
|---|---|---|
| Acrylic acid | 1.0000 | 1.0000 |
| Water | 0.5000 | 3.0000 |
| Hydrophobic solvent | 1.2000 | 12.0000 |
| Base (expressed | 0.6600 | 1.1100 |
| as 50% NaOH) | (60% neutral) | (100% neutralized) |
| Crosslinker | 0.0030 | 0.0080 |
| Initiator | 0.0005 | 0.0200 |
| Chelating agent | 0.0000 | 0.0050 |
| Surfactant | 0.0050 | 0.0400 |

An exemplary inverse suspension reaction to form a crosslinked polymer may involve preparation of two mixtures (e.g., a hydrophobic mixture and an aqueous mixture) in two different vessels followed by combination of the mixtures to form a reaction mixture. One vessel may be designated as a hydrophobic compound vessel and the other may be designated as an aqueous solution vessel. The hydrophobic compounds may be mixed in a larger vessel that will become a reaction vessel, while an aqueous solution may be prepared in a smaller vessel that may be discharged into the reaction vessel. In an exemplary embodiment, the hydrophobic mixture may contain solvent, surfactant, and crosslinking agent, and the aqueous mixture may contain water, base, monomer (e.g., acrylic acid), initiator, and optional chelating agent.

A hydrophobic solvent may be introduced into the reaction vessel. As will be appreciated by one of skill in the art, a hydrophobic solvent (also referred to herein as the “oil phase”) may be chosen based upon one or more considerations, including, for example, the density and viscosity of the oil phase, the solubility of water in the oil phase, the partitioning of the neutralized and unneutralized ethylenically unsaturated monomers between the oil phase and the aqueous phase, the partitioning of the crosslinker and the initiator between the oil phase and the aqueous phase and/or the boiling point of the oil phase.

Hydrophobic solvents contemplated for use in the present disclosure include, for example, Isopar™ L (isoparaffin fluid), toluene, benzene, dodecane, cyclohexane, n-heptane and/or cumene. Preferably, Isopar™ L is chosen as a hydrophobic solvent due to its low viscosity, high boiling point and low solubility for neutralized monomers such as sodium acrylate and/or potassium acrylate. One of skill in the art will appreciate that a large enough volume of hydrophobic solvent is used to ensure that the aqueous phase is suspended as droplets in the oil rather than the reverse and that the aqueous phase droplets are sufficiently separated to prevent coalescence into large masses of aqueous phase.

One or more surfactants and one or more crosslinkers may be added to the oil (hydrophobic) phase. The oil phase may then be agitated and sparged with an inert gas, such as nitrogen or argon to remove oxygen from the oil phase. It will be appreciated that the amount of surfactant used in the reaction depends on the size of the desired polymer particles and the agitator stir rate. This addition of surfactant is designed to coat the water droplets formed in the initial reaction mixture before the reaction starts. Higher amounts of surfactant and higher agitation rates produce smaller droplets with more total surface area. It will be understood by those of skill in the art that an appropriate choice of cross-linker and initiator may be used to prepare spherical to ellipsoid shaped beads. One of skill in the art will be capable of determining an appropriate cross-linker for the preparation of a specified cross-linked cation-binding polymer. For example: cross-linker choice depends on whether it needs to be hydrophobic or hydrophilic polymer or whether it needs to resist acidic or basic external conditions. An amount of cross-linker depends on how much soluble polymer is permissible and how much saline holding capacity is desired.

Exemplary surfactants include hydrophobic agents that are solids at room temperature, including, for example, hydrophobic silicas (such as Aerosil® or Perform-O-Sil™) and glycolipids (such as polyethylene glycol distearate, polyethylene glycol dioleate, sorbitan monostearate, sorbitan monooleate or octyl glucoside).

Crosslinking agents with two or more vinyl groups that are not in resonance with each other may be used, allowing for a wide variety in molecular weight, aqueous solubility and/or lipid (e.g., oil) solubility. Crosslinking agents contemplated for use in the present disclosure, include, for example, diethyleneglycol diacrylate (diacryl glycerol), triallylamine, tetraallyloxyethane, allylmethacrylate, 1,1,1-trimethylolpropane triacrylate (TMPTA), and divinylbenzene.

In some embodiments, the crosslinker is one or more compound having (in one molecule) 2-4 groups selected from the group consisting of CH₂═CHCO—, CH═C(CH₃)CO— and CH₂═CH—CH₂—, for example and without limitation: diacrylates and dimethacrylates of ethylene glycol, diethylene glycol, triethylene glycol, propylene glycol, 1,4-butanediol, 1,5-pentanediol, 1,6-hexanediol, neopentyl glycol, trimethylol propane, pentaerythritol, triacrylates and trimethacrylates of trimethylolpropane and pentaerythritol, highly ethoxylated trimethylol propane triacrylate, tetracrylate and tetramethacrylate of pentaerythritol, allyl methacrylate, and tetraallyloxyethane.

In some embodiments, a heat activated crosslinker may be used in the preparation of crosslinked polymers according to the present disclosure. Non-limiting examples of heat-activated crosslinkers include hydroxyl-containing crosslinking agents containing at least one hydroxyl functionality suitable to react with a carboxyl group on the polymer and containing at least two functional groups capable of forming covalent bonds with the polymer. Some non-limiting examples of heat-activated crosslinkers suitable for such use is the class of compounds commonly referred to as polyols or polyhydroxy compounds. Some non-limiting examples of polyols include: glycerin, ethylene glycol, diethylene glycol, triethylene glycol, propylene glycol, 1,4-butanediol, 1,5-pentanediol, 1,6-hexanediol, neopentyl glycol, polyglycerin, trimethylolpropane, polyethylene glycol, and polypropylene glycol-polyethylene glycol copolymers.

In some embodiments, dimodal crosslinkers may be used in the preparation of crosslinked polymers according to the present disclosure. Dimodal crosslinkers contain one or more hydroxyl groups and one or more ethylenically unsaturated groups in the same compound. Non-limiting examples of dimodal crosslinkers suitable for use to crosslink polymers according to the present disclosure include: 2-hydroxyethyl(meth)acrylate, polyethylene glycol monomethacrylate, glycidyl methacrylate, allyl glycidyl ether, hydroxypropyl methacrylate, hydroxyethyl methacrylate, and hexapropylene glycol monomethacrylate.

In some embodiments, polyvinyl compounds may be used in the preparation of crosslinked polymers according to the present disclosure. Non-limiting examples of polyvinyl crosslinkers include divinyl compounds or polyvinyl compounds such as: divinyl benzene, divinyl toluene, divinyl xylene, divinyl ether, divinyl ketone, trivinyl benzene; diesters or polyesters of unsaturated monocarboxylic acids or polycarboxylic acids with polyols, such as: di(meth)acrylic acid esters or tri(meth)acrylic acid esters of polyols such as ethylene glycol, diethylene glycol, triethylene glycol, tetra ethylene glycol, propylene glycol, dipropylene glycol, tri propylene glycol, tetra propylene glycol, trimethylol propane, glycerin, polyoxyethylene glycols and polyoxypropylene glycols; unsaturated polyesters that can be obtained by reacting any of the above-mentioned polyols with an unsaturated acid such as maleic acid; diesters or polyesters of unsaturated mono- or polycarboxylic acids with polyols derived from reaction of C₂-C₁₀ polyhydric alcohols with 2-8 C₂-C₄ alkylene oxide units per hydroxyl group, such as trimethylol propane hexaethoxyl triacrylate; di-methacrylic acid or tri-methacrylic acid esters that can be obtained by reacting polyepoxide with methacrylic acid; bis(meth)acrylamides such as N,N-methylene-bisacrylamide; carbamyl esters that can be obtained by reacting polyisocyanates, such as tolylene diisocyanate, hexamethylene diisocyanate, 4,4′-diphenyl methane diisocyanate; and NCO-containing prepolymers obtained by reacting such diisocyanates with active hydrogen atom-containing compounds with hydroxyl group-containing monomers, such as di-methacrylic acid carbamyl esters obtainable by reacting the above-mentioned diisocyanates with hydroxyethyl(meth)acrylate; di(meth)allyl ethers or poly(meth)allyl ethers of polyols such as alkylene glycols, glycerol, polyalkylene glycols, polyoxyalkylene polyols and carbohydrates such as polyethylene glycol diallyl ether, allylated starch, and allylated cellulose; di-allyl or poly-allyl esters of polycarboxylic acids, such as diallyl phthalate and diallyl adipate; and esters of unsaturated monocarboxylic acids or polycarboxylic acids with mono(meth)allyl ester of polyols, such as allyl methacrylate or (meth)acrylic acid ester of polyethylene glycol monoallyl ether. In some embodiments, the crosslinker may be one or more compound consistent with the following formula:

R¹—(—(R²⁰)ₙ—C(O)R³)ₓ,

wherein:

R¹ is a straight-chain or branched-chain C₁-C₁₀ polyalkoxy radical, optionally substituted with one or more oxygen atoms in the backbone, having x valences;

each R² is independently a C₂-C₄ alkylene group;

each R³ is independently a straight-chain or branched-chain C₂-C₁₀ alkenyl moiety;

n is a positive integer from 1-20; and

x is a positive integer from 2-8.

An aqueous phase mixture may be prepared in another vessel (e.g., a vessel that is separate from that used to prepare the hydrophobic phase) that contains water. For example, preparation of neutralized or partially neutralized polymer, base and monomer are added to the water. For preparation of non-neutralized (acid form) polymer, monomer is added to the water without base. It will be appreciated by one of skill in the art that the amount of base used in the vessel is determined by the degree of neutralization of the monomer desired. For neutralized or partially neutralized polymer, a degree of neutralization between about 60% and 100% is preferred. Without wishing to be bound by a theory or mechanism, it is believed that one-hundred percent neutralization minimizes the chance of suspension failure, but the highly charged monomer may not react as rapidly and may not pull hydrophobic crosslinkers into the forming polymer. Considerations in choosing the degree of neutralization may be determined by one of skill in the art and include, for example, the effect of monomer charge (e.g., as determined by ionization of the cation from the neutralized molecules) on reaction rate, partitioning of the monomer and neutralized monomer between oil phase and aqueous phase and/or tendency of the aqueous droplets to coalesce during the reaction. The solubilities of sodium acrylate and sodium methacrylate in water are limited and are lower at lower temperatures (e.g., sodium acrylate is soluble at about 45% at 70° C. but less than 40% at 20° C.). This solubility may establish the lower limit of the amount of water needed in the neutralization step. The upper limit of the amount of water may be based on reactor size, amount of oil phase needed to reliably suspend the aqueous phase as droplets and/or the desired amount of polymer produced per batch.

Bases contemplated for use in methods of making the crosslinked polymers of the present disclosure include, for example, hydroxides, bicarbonates, or carbonates. Use of these bases allows neutralization of the acid monomer without residual anions left in the reaction mixture as the anions react to form water or CO₂. Frequently, sodium bases are chosen in the method of making the crosslinked polymers. However, potassium bases, ammonium bases, and bases of other cations, including calcium bases, are contemplated for use in the present disclosure.

The water used in the reaction may be purified water or water from other sources such as city water or well water. If the water used is not purified water, chelating agents may be needed to control metals, e.g., heavy metal ions, such as iron, calcium, and/or magnesium from destroying the initiator. Chelating agents contemplated for use with the present disclosure include, for example, diethylenetriaminepentaacetic acid pentasodium (Versenex™ 80). The amount of chelating agent added to the reaction mixture may be to determined by one of skill in the art from a determination of the amount of undesirable metal in the water.

Once base is added to the water, the aqueous phase solution may be cooled to remove the heat released from dilution of the base, and one or more classes of monomers may be added, to react with the base, for example, monomers which will be neutralized by the base. As will be appreciated by one of skill in the art, the monomers will be neutralized to the degree dictated by the amount of base in the reaction. The aqueous phase solution may be kept cool (e.g., below 35 to 40° C.) and preferably around 20° C. to prevent formation of prepolymer strands, dimers and/or possible premature polymerization.

Monomers are dissolved in water at concentrations of 10-70 wt % or 20-40 wt % and polymerization may subsequently be initiated by free radicals in the aqueous phase. Monomers may be polymerized either in the acid form (pH 2-4) or as a partially neutralized salt (pH 5-7). For an inverse suspension process, monomers in the acid form may be less desirable due to high solubility in the oil phase. The amount of water used to dissolve the monomer is minimally set so that all of the monomer (e.g., sodium acrylate) is dissolved in the water rather than crystallizing and maximally set so that there is the smallest volume of reaction mixture possible (to minimize the amount of distillation and allow the maximum yield per batch).

Exemplary monomer units contemplated for use in the present disclosure, include, for example, acrylic acid and its salts, methacrylic acid and its salts, crotonic acid and its salts, tiglinic acid and its salts, 2-methyl-2-butenoic acid (Z) and its salts, 3-butenoic acid (vinylacetic acid) and its salts, 1-cyclopentene carboxylic acid, and 2-cyclopentene carboxylic acid and their salts; and unsaturated dicarboxylic acids and their salts, such as maleic acid, fumaric acid, itaconic acid, glutaconic acid, and their salts. Other cross-linked polyelectrolyte superabsorbent polymers may be based on sulfonic acids and their salts, phosphonic acids and their salts, or amines and their salts.

One or more initiators, such as free radical producers, may be added to the aqueous phase just before the aqueous phase is transferred into the oil phase. As will be appreciated by one of skill in the art, the initiator amount and type used in the polymerization reaction depends on oil versus water solubility and whether longer chain lengths are desired. For example, a lower amount of initiator may be used in the polymerization reaction when longer chain lengths are desired.

In some embodiments, the initiator may be a thermally sensitive compound such as a persulfate, 2,2′-azobis(2-amidino-propane)-dihydrochloride, 2,2′-azobis (2-amidino-propane)-dihydrochloride and/or 2,2′-azobis (4-cyanopentanoic acid). Thermally sensitive initiators have the disadvantage that the polymerization does not begin until an elevated temperature is reached. For persulfates, this temperature is approximately 50 to 55° C. Since the reaction is highly exothermic, vigorous removal of the heat of reaction is required to prevent boiling of ° the aqueous phase. It is preferred that the reaction mixture be maintained at approximately 65° C. As will be appreciated by one of skill in the art, thermal initiators have the advantage of allowing control of the start of the reaction when the reaction mixture is adequately sparged of oxygen.

In some embodiments, the initiator may also be a redox pair such as persulfate/bisulfate, persulfate/thiosulfate, persulfate/ascorbate, hydrogen peroxide/ascorbate, sulfur dioxide/tert-butylhydroperoxide, persulfate/erythorbate, tert-butylhydroperoxide/erythorbate and/or tert-butylperbenzoate/erythorbate. These initiators are able to initiate the reaction at room temperature, thereby minimizing the chance of heating the reaction mixture to the boiling point of the aqueous phase as heat is removed through the jacket around the reactor. However, homogeneous mixing may not accomplished by the time the reaction is initiated and there may be rapid polymerization of the surface of the droplets with much slower polymerization within the material.

In some embodiments, the reaction is not started immediately after the mixing of the aqueous phase into the oil phase in the final reactor because the aqueous phase still has an excessive amount of oxygen dissolved in the water. It will be appreciated by one of skill in the art that an excessive amount of oxygen may cause poor reactivity and inadequate mixing may prevent the establishment of uniform droplet sizes. Instead, the final reaction mixture is first sparged with an inert gas for ten to sixty minutes after all reagents (except the redox pair if that initiator system is used) have been placed in the reactor. The reaction may be initiated when a low oxygen content (e.g., below 15 ppm) is measured in the inert gas exiting the reactor.

It will be appreciated by those of skill in the art that with acrylate and methacrylate monomers, polymerization begins in the droplets and progresses to a point where coalescence of the particles becomes more likely (the “sticky phase”). It may be necessary that a second addition of surfactant (e.g., appropriately degassed to remove oxygen) be added during this phase or that the agitation rate be increased. For persulfate thermal initiation, this sticky phase may occur at about 50 to 55° C. For redox initiation systems, the need for additional surfactant may be lessened by the initial surface polymerization, but if additional surfactant is needed, it should be added as soon as an exotherm is noted.

The reaction may be continued for four to six hours after the peak exotherm is seen to allow for maximal consumption of the monomer into the polymer. Following the reaction, the polymeric material may be isolated by either transferring the entire reaction mixture to a centrifuge or filter to remove the fluids or by initially distilling the water and some of the oil phase (e.g., frequently as an azeotrope) until no further removal of water is possible and the distillation temperature rises significantly above 100° C., followed by isolating the polymeric material by either centrifugation or filtering. The isolated crosslinked cation-binding polymeric material is then dried to a desired residual moisture content (e.g., less than 5%).

An exemplary cross-linked cation-binding polymer, polyacrylate, may be formed by copolymerizing an ethylenically unsaturated carboxylic acid with a multifunctional cross-linking monomer. The acid monomer or polymer may be substantially or partially neutralized with an alkali metal salt such as a hydroxide, a carbonate, or a bicarbonate and polymerized by the addition of an initiator. One such exemplary polymer gel is a copolymer of acrylic acid/sodium acrylate and any of a variety of cross-linkers.

The reactants for the synthesis of exemplary cross-linked cation-binding polymers, such as cross-linked polyacrylate, are provided in Table 2 below. These cross-linked cation-binding polymer may be produced as a one-hundred kilogram batch in a five-hundred gallon vessel.

TABLE 2
List of Components Used in the Manufacture
of Cross-linked Polyacrylate Polymer

| | | Amount/batch |
|---|---|---|
| Component | Function | (kg) |
| Acrylic Acid | Monomer | 88 |
| Water | Solvent | 90 |
| 50% Sodium Hydroxide | Neutralization of | 79 |
| | acrylic acid monomer | |
| Naphtha [petroleum], | Continuous phase | As needed |
| hydrotreated heavy, | for Suspension | |
| (Isopar ™ L) | | |
| Fumed silica (Aerosil R972) | Suspending agent | 0.9 |
| | (Surfactant) | |
| Diethylenetriaminepentaacetic | Control of metal | 0.9 |
| Acid Pentasodium | ions in reagents, | |
| (Versenex ™ 80) | solvents, or | |
| Sodium Persulfate | Polymerization | 0.06 |
| | initiator | |
| Trimethylolpropane | Cross-linking agent | 0.3 |
| Triacrylate, (TMPTA) | | |

An exemplary polymerization reaction is shown below.

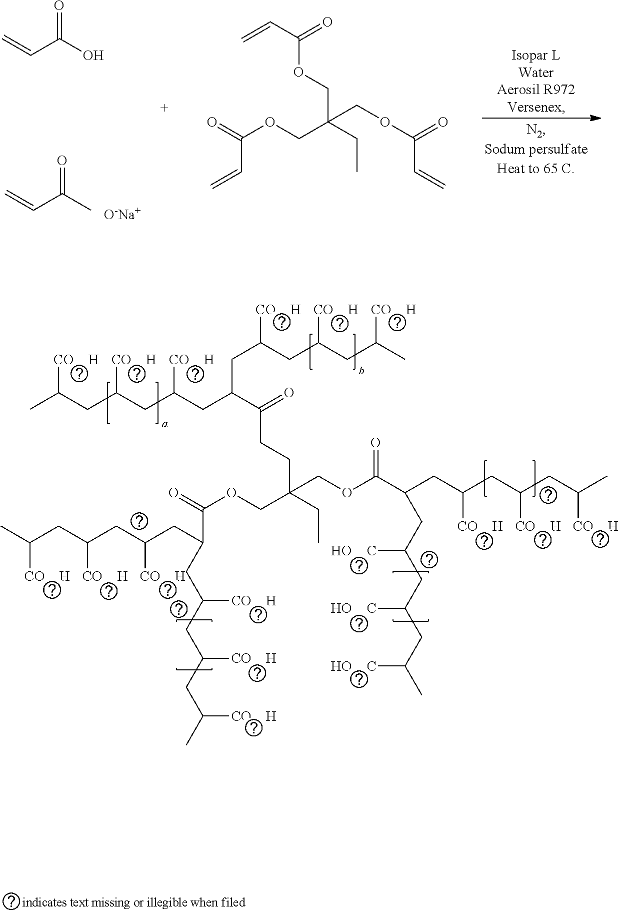

2. Preparation of Crosslinked Cation-Binding Polymers with Hydrogen Counterions

Partially neutralized or fully neutralized crosslinked cation-binding polymers may be acidified by washing the polymer with acid. Suitable acids contemplated for use with the present disclosure, include, for example, hydrochloric acid, acetic acid and phosphoric acid.

Those skilled in the art will recognize that the replacement of the counterions, including cations such as sodium atoms; by hydrogen atoms can be performed with many different acids and different concentrations of acid. However, care must be taken in choice of acid and concentration to avoid damage to the polymer or the cross-linkers. For instance, nitric and sulfuric acids would be avoided.

Acid-washed crosslinked cation-binding polymers may be additionally rinsed with water and then dried in, for example, a vacuum oven or inert atmosphere until less than 5% moisture remains, to produce cross-linked polyacrylic acid which is substantially the free acid form of cross-linked polyacrylic acid. Any particle form of partially or fully to neutralized cross-linked cation-binding polymer may be used as the starting point, for example, granular powders, or bead-form particles, for example, from an inverse suspension process as described above. Optionally, if the intact bead form of partially or fully neutralized cross-linked polyacrylate is used, the acid-washed cross-linked polyelectrolyte polymer may be left in the bead form as recovered from the oven or may be additionally milled to obtain smaller particles of the cross-linked polyelectrolyte polymer, for example, low-sodium cross-linked polyelectrolyte polymer.

Alternatively, crosslinked cation-binding polymers may be prepared from monomers with unneutralized carboxylic acid groups. For example, a crosslinked polyacrylate can be prepared from acrylic acid without first neutralizing with a base. A monomer solution is prepared in a reactor by dissolving an unsaturated carboxylic acid monomer (e.g., acrylic acid) in water. Optionally, a chelating agent (e.g., Versenex™ 80) may be added to control metal ions. A suitable crosslinking agent (e.g., trimethylolpropane triacrylate or diacryl glycerol) is added to the reactor. Choice of crosslinkers is the same as previously described herein. The temperature of the monomer solution is adjusted as desired. A polymerization initiator is added to the reactor. The reactor is then closed and the reaction mixture is bubbled with an inert gas (e.g., nitrogen) and agitated until adequate removal of oxygen is achieved. The reaction is then initiated either by reaching an oxygen concentration where a redox couple (e.g., tertiary butylhydroperoxide/thiosulfate, or hydrogen peroxide/erythorbic acid) produces radicals that are not quenched by oxygen, or by adding heat to cause a temperature dependent initiator (e.g., sodium persulfate) to produce radicals. Alternatively, the monomer solution is deoxygenated prior to the addition of the initiators. The reaction is allowed to proceed through the exothermic heating that occurs during reaction. Reaction heat can be removed and controlled as desired by methods known to those skilled in the art. After about 2 to 6 hours, the reaction is completed and the gel-like mass of reaction product can be removed from the reactor and cut into appropriately sized pieces. After drying, the particles can be separated by size or milled to produce the desired size. Other examples of the polymerization of aqueous acrylic acid solutions with crosslinkers are disclosed in U.S. Pat. No. 4,654,039; U.S. Pat. No. 4,295,987; U.S. Pat. No. 5,145,906; and U.S. Pat. No. 4,861,849, the contents of which are incorporated herein by reference.

Exemplary crosslinked cation-binding polymers, including for example those prepared according to Examples 1-5, generally have a saline holding capacity of greater than about 40 g/g (see, e.g., Examples 8 and 9); and contain less than about 5,000 ppm of sodium, less than about 20 ppm of heavy metals, less than about 500 ppm of residual monomer, less than about 2,000 ppm of residual chloride, and less than about 20 wt. % of soluble polymer. Preferably, acidified polymers useful as crosslinked cation-binding polymers prepared according to this Example have a saline holding capacity of greater than about 80 g/g (see, e.g., Examples 8 and 9); and contain less than about 500 ppm of sodium, less than about 20 ppm of heavy metals, less than about 50 ppm of residual monomer, less than about 1,500 ppm of residual chloride, and less than about 10 wt. % of soluble polymer.

Crosslinked cation-binding polymers prepared according to the method of Examples 1 or 2 using acrylic acid monomers, followed by acidification according to Example 3, or crosslinked cation-binding polymers prepared according to Example 4, are referred to as H—CLP or HCLP. Crosslinked cation-binding polymers comprising calcium or magnesium may be referred to as Ca—CLP or CaCLP, or Mg—CLP or MgCLP, respectively. For example, Ca—CLP may be prepared according to the method of Example 5 or 7.

3. Preparation of Crosslinked Cation-Binding Polymers with Increased Saline Holding Capacity

Partially neutralized or non-neutralized (e.g., acidified) crosslinked cation-binding polymers of the present disclosure, including cross-linked polyacrylate and/or polyacrylic acid polymers, may be disrupted (e.g., milled) to increase their saline holding capacity. Saline holding capacity may be determined, for example, as described in Example 8 and 9.

Crosslinked cation-binding polymer may be disrupted into smaller particles, for example, by milling or grinding. The disrupted polymeric material is preferably washed to remove impurities such as soluble polymer, residual monomer, and/or other impurities. Suitable washing solutions include purified water such as deionized water or distilled water and various alcohols, or mixtures of water and various alcohols. Since the polymer is to be dried, it is desirable to use fluids that will evaporate easily without leaving any residue, such as salts, in the dried polymer. Alternatively, cross-linked cation-binding polymer, including cross-linked polyacrylate polymeric beads, may be disrupted, for example, to reduce impurities, by placing the polymer into purified water or other solvent and agitating the polymer (e.g., stirring with a magnetic stir bar or agitating at 500 rpm overnight). The residual soluble polymer in the crosslinked polymers may thus be reduced or eliminated and the saline holding capacity of the polymeric material (e.g., reported per gram of polymer) increased.

In embodiments where the crosslinked cation-binding polymer is prepared from unneutralized monomers, such as acrylic acid, the bulk polymer may first be cut into pieces and dried (e.g., in a vacuum oven) before milling or grinding.

After milling or grinding of the crosslinked polymer, particles of a certain size, e.g., particles of a desired size or a particle size distribution characterized, for example, by an average size, may be obtained by means known to those of skill in the art, for example, by sieving through sieves such as screens. Screens may be stacked to obtain particles with a range of sizes. Screens are shaken to allow particles to sift through and get caught on the screen with an opening just below their diameter. For example, particles that pass through an 18 Mesh screen and are caught on a 20 Mesh screen are between 850 and 1000 microns in diameter. Screen mesh and the corresponding particle size allowed to pass through the mesh include, 18 mesh, 1000 microns; 20 mesh, 850 microns; 25 mesh, 710 microns; 30 mesh, 600 microns; 35 mesh, 500 microns, 40 mesh, 425 microns; 45 mesh, 35 microns; 50 mesh, 300 microns; 60 mesh, 250 microns; 70 mesh, 212 microns; 80 mesh, 180 microns; 100 mesh, 150 microns; 120 mesh, 125 microns; 140 mesh, 106 microns; 170 mesh, 90 microns; 200 mesh, 75 microns; 230 mesh, 63 microns; and 270 mesh, 53 microns. Thus particles of varying sizes may be obtained through the use of one or more screens.

### Compositions, Formulations, and Dosage Forms

Compositions, formulations, and/or dosage forms, e.g., pharmaceutical compositions, formulations, and/or dosage forms, are disclosed comprising a cross-linked cation-binding polymer comprising monomers containing carboxylic acid groups (e.g., a cross-linked polyacrylic acid polymer) and wherein the polymer further comprises calcium and/or magnesium cations, wherein the calcium cations are counterions to about 15% to about 35% of the carboxylic acid groups in the polymer (alternately, the polymer comprises calcium and/or magnesium counterions to about 15% to about 30%, about 20% to about 30%, or about 25% to about 35% of the carboxylate groups in the polymer). In some embodiments, the polymer comprises calcium and/or magnesium cations that are counterions to about 15%, about 20%, about 25%, about 30%, or about 35% of the carboxylate groups in the polymer. In related embodiments, the calcium and/or magnesium cations are counterions to about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% of the carboxylate groups in the polymer. These polymers, compositions, formulations, and/or dosage forms may be delivered to a subject, including using a wide variety of routes or modes of administration. Preferred routes for administration are oral or intestinal.

In some embodiments, a composition, formulation, or dosage form as disclosed herein comprises a polymer that comprises calcium cations as counterions to about 15% to about 35% of the carboxylate groups on the polymer, and sodium cations as counterions to no more than about 5% of the carboxylate groups on the polymer. In such embodiments, the polymer further comprises hydrogen cations (e.g., protons) as counterions to all or substantially all of the carboxylate groups to which calcium and sodium are not counterions (e.g., “free carboxylates”), for example about 95% of the free carboxylates, about 96% of the free carboxylates, about 97% of the free carboxylates, about 98% of the free carboxylates, about 99% of the free carboxylates, about 99.5% of the free carboxylates, or about 100% of the free carboxylates. In some embodiments, the polymer comprises calcium cations as counterions to about 25% of the carboxylate groups on the polymer, sodium cations as counterions to no more than about 5% of the carboxylate groups on the polymer, and hydrogen cations (e.g., protons) as counterions to all or substantially all of the free carboxylates.

In some embodiments, a composition, formulation, or dosage form as disclosed herein comprises a polymer that comprises magnesium cations as counterions to about 15% to about 35% of the carboxylate groups on the polymer and sodium cations as counterions to no more than about 5% of the carboxylate groups on the polymer, and further comprises hydrogen cations (e.g., protons) as counterions to all or substantially all of the carboxylate groups to which magnesium or sodium are not counterions (e.g., “free carboxylates”), for example about 95% of the free carboxylates, about 96% of the free carboxylates, about 97% of the free carboxylates, about 98% of the free carboxylates, about 99% of the free carboxylates, about 99.5% of the free carboxylates, or about 100% of the free carboxylates. In some embodiments, the polymer comprises calcium cations as counterions to about 25% of the carboxylate groups on the polymer, sodium cations as counterions to no more than about 5% of the carboxylate groups on the polymer, and hydrogen cations (e.g., protons) as counterions to all or substantially all of the free carboxylates.

In some embodiments, a composition, formulation, or dosage form as described herein comprises a crosslinked cation-binding polymer comprising repeat units containing carboxylic acid groups, and wherein the polymer further comprises calcium and/or magnesium cations that are counterions to about 15% to about 35% of the carboxylate groups in the polymer (alternatively, to about 15% to about 30%, about 20% to about 30%, or about 25% to about 35% of the carboxylate groups in the polymer) and further comprises an added base, wherein the base is present in an amount sufficient to provide up to about 0.8 equivalents of base per equivalent of carboxylic acid groups in the polymer. In a related example the composition, formulation, or dosage form contains about 0.05 equivalents, about 0.1 equivalents, about 0.15 equivalents, about 0.2 equivalents, about 0.25 equivalents, about 0.3 equivalents, about 0.35 equivalents, about 0.4 equivalents, about 0.45 equivalents, about 0.5 equivalents, about 0.55 equivalents, about 0.6 equivalents, about 0.65 equivalents, about 0.7 equivalents, about 0.75 equivalents, or about 0.8 equivalents of base per equivalent of carboxylic acid groups in the polymer.

In some embodiments, a composition, formulation, and/or dosage form as described herein comprises a crosslinked cation-binding polymer comprising carboxylic acid-containing monomers and calcium and/or magnesium cations, wherein the calcium and/or magnesium cations are present in an amount sufficient to act as counterions to about 15% to about 35% of the carboxylate groups in the polymer, and an amount of added base sufficient to provide up to about 0.8 equivalents of base per equivalent of carboxylate groups in the polymer (alternatively, about 0.2 to about 0.8, about 0.3 to about 0.6, or about 0.35 to about 0.5 equivalents of base per equivalent of carboxylate groups in the polymer; alternatively, the base is present in an amount sufficient to provide about 0.65 to about 0.75, such as about 0.66, about 0.70, about 0.73, about 0.74 equivalents of base per equivalent of carboxylate groups in the polymer). In related embodiments, the calcium and/or magnesium cations are present in an amount sufficient to act as counterions to about 15% to about 30% of the carboxylate groups in the polymer, and added base is present in an amount sufficient to provide up to about 0.8 equivalents of base per equivalent of carboxylate groups in the polymer (alternatively, about 0.3 to about 0.6 or about 0.35 to about 0.5 equivalents of base per equivalent of carboxylate groups in the polymer). In related embodiments, the calcium and/or magnesium cations are present in an amount sufficient to act as counterions to about 20% to about 30% of the carboxylate groups in the polymer, and added base is present in an amount sufficient to provide up to about 0.8 equivalents of base per equivalent of carboxylate groups in the polymer (alternatively, about 0.2 to about 0.8, 0.3 to about 0.6 or about 0.35 to about 0.5 equivalents of base per equivalent of carboxylate groups in the polymer). In related embodiments, the calcium and/or magnesium cations are present in an amount sufficient to act as counterions to about 25% to about 35% of the carboxylate groups in the polymer, and added base is present in an amount sufficient to provide up to about 0.8 equivalents of base per equivalent of carboxylate groups in the polymer (alternatively, about 0.2 to about 0.8, about 0.3 to about 0.6, or about 0.35 to about 0.5 equivalents of base per equivalent of carboxylate groups in the polymer).

The polymers in any of the compositions, formulations, and/or dosage forms disclosed herein may comprise sodium cations which (if present) are counterions to no more than about 5%, 4%, 3%, 2%, 1%, or 0.5% of the carboxylate groups in the polymer.

In some embodiments, the polymers disclosed herein for inclusion in a composition, formulation, or dosage form, e.g., for administration to an individual, e.g., for use in methods of treatment disclosed herein, are individual particles or particles agglomerated to form a larger particle (for example, flocculated particles), and have a diameter of about 1 to about 10,000 microns (alternatively, about 1 micron to about 50 microns, about 10 microns to about 50 microns, about 10 microns to about 200 microns, about 50 microns to about 100 microns, about 50 microns to about 200 microns, about 50 microns to about 1000 microns, about 500 microns to about 1000 microns, about 1000 to about 5000 microns, or about 5000 microns to about 10,000 microns). In some embodiments, the particles or agglomerated particles have a diameter of about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 7000, about 7500, about 8000, about 8500, about 9000, about 9500, or about 10,000 microns.

In some embodiments, the crosslinked cation-binding polymer disclosed herein for inclusion in a composition, formulation, or dosage form, e.g., for administration to an individual, e.g., for use in methods of treatment disclosed herein is a crosslinked polyacrylate polymer. For example, the polymer may be a polyacrylate polymer crosslinked with about 0.08 mol % to about 0.2 mol % crosslinker, and for example, may comprise an in vitro saline absorption capacity of at least about 20 times its weight (e.g., at least about 20 grams of saline per gram of polymer, or “g/g”), at least about 30 times its weight, at least about 40 times its weight, at least about 50 times its weight, at least about 60 times its weight, at least about 70 times its weight, at least about 80 times its weight, at least about 90 times its weight, at least about 100 times its weight, or more. In some embodiments, the crosslinked polyacrylate polymer is in the form of individual particles or particles that are agglomerated (for example, flocculated) to form a larger particle, wherein the diameter of individual particles or agglomerated particles is about 1 micron to about 10,000 microns (alternatively, about 1 micron to about 10 microns, about 1 micron to about 50 microns, about 10 microns to about 50 microns, about 10 microns to about 200 microns, about 50 microns to about 100 microns, about 50 microns to about 200 microns, about 50 microns to about 1000 microns, about 500 microns to about 1000 microns, about 1000 to about 5000 microns, or about 5000 microns to about 10,000 microns. In one embodiment, the polyacrylate polymer is in the form of small particles that flocculate to form agglomerated particles with a diameter of about 1 micron to about 10 microns.

In some embodiments in which a composition, formulation, or dosage form comprises added base, the added base component is one or more of: an alkali metal hydroxide, an alkali metal acetate, an alkali metal carbonate, an alkali metal bicarbonate, an alkali metal oxide, an alkali earth metal hydroxide, an alkali earth metal acetate, an alkali earth metal carbonate, an alkali earth metal bicarbonate, an alkali earth metal oxide, an organic base, choline, lysine, arginine, histidine, an acetate, a butyrate, a propionate, a lactate, a succinate, a citrate, an isocitrate, a fumarate, a malate, a malonate, an oxaloacetate, a pyruvate, a phosphate, a carbonate, a bicarbonate, a lactate, a benzoate, a sulfate, a lactate, a silicate, an oxide, an oxalate, a hydroxide, an amine, a dihydrogen citrate, calcium bicarbonate, calcium carbonate, calcium oxide, calcium hydroxide, magnesium oxide, magnesium carbonate, magnesium hydrochloride, sodium bicarbonate, and potassium citrate, or a combination thereof. In one embodiment, the added base is calcium carbonate.

In some embodiments, a composition, formulation, or dosage form disclosed herein comprises a polyacrylate polymer that comprises calcium and/or magnesium counterions to about 15% to about 35% of the carboxylate groups in the polymer, for example, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% of the carboxylate groups in the polymer, and wherein sodium cations (if present) are counterions to no more than about 5% of the carboxylate groups in the polyacrylate polymer. In some embodiments, the composition, formulation, or dosage form additionally comprises added base, for example, calcium carbonate base, present in an amount to provide up to about 0.8 equivalents of base per carboxylate group on the polyacrylate polymer, for example, about 0.05 equivalents, 0.1 equivalents, 0.15 equivalents, 0.2 equivalents, 0.25 equivalents, 0.3 equivalents, 0.35 equivalents, 0.4 equivalents, 0.45 equivalents, 0.5 equivalents, 0.55 equivalents, 0.6 equivalents, 0.65 equivalents, 0.7 equivalents, 0.75 equivalents, or 0.8 equivalents of base per equivalent of carboxylate groups in the polyacrylate polymer

In some embodiments, the above compositions, formulations, and/or dosage forms additionally comprise one or more excipients, carriers, or diluents. Compositions for use in accordance with the present disclosure may be formulated in conventional manner using one or more physiologically acceptable carriers comprising excipients and auxiliaries which facilitate processing of the polymer into preparations which may be used pharmaceutically. Proper formulation is dependent upon the route of administration chosen. Such compositions may contain a therapeutically effective amount of polymer and may include a pharmaceutically acceptable carrier. Pharmaceutically acceptable carriers include those approved by a regulatory agency of the Federal or a state government or listed in the U.S. Pharmacopeia or other generally recognized pharmacopeia for use in animals, and more particularly, in humans. Carriers can include an active ingredient in which the disclosed compositions are administered.

In some embodiments, the composition, formulation, or dosage form is in the form of a tablet, a chewable tablet, a capsule, a suspension, an oral suspension, a powder, a gel block, a gel pack, a confection, a chocolate bar, a pudding, a flavored bar, or a sachet. In some embodiments, the composition, formulation, or dosage form contains about 1 g to about 30 g or about 100 g of a disclosed cation-binding polymer. For example and without limitation, the composition, formulation, or dosage form may include about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g, about 25 g, about 26 g, about 27 g, about 28 g, about 29 g, about 30 g, about 35 g, about 40 g, about 45 g, about 50 g, about 55 g, about 60 g, about 65 g, about 70 g, about 75 g, about 80 g, about 85 g, about 90 g, about 95 g, or about 100 g, or more of the cation-binding polymer. Regardless of the amount of polymer present in the composition, formulation, or dosage form, a composition, formulation, or dosage form of the present disclosure may optionally also include up to about 0.8 equivalents of base, for example, a pharmaceutically and/or physiologically acceptable base, per equivalent of carboxylate groups in the polymer, for example, about 0.05 equivalents, about 0.1 equivalents, about 0.15 equivalents, 0.2 equivalents, about 0.25 equivalents, about 0.3 equivalents, about 0.35 equivalents, about 0.4 equivalents, about 0.45 equivalents, about 0.5 equivalents, about 0.55 equivalents, about 0.6 equivalents, about 0.65 equivalents, about 0.7 equivalents, about 0.75 equivalents, or about 0.8 equivalents of base per equivalent of carboxylic acid groups in the polymer. In some embodiments, the base is present in an amount sufficient to provide from about 0.2 equivalents to about 0.8 equivalents of base, for example about 0.2 equivalents, about 0.25 equivalents, about 0.3 equivalents, about 0.35 equivalents, about 0.4 equivalents, about 0.45 equivalents, about 0.5 equivalents, about 0.55 equivalents, about 0.6 equivalents, about 0.65 equivalents, about 0.7 equivalents, about 0.75 equivalents, or about 0.8 equivalents of base per equivalent of carboxylate groups in the polymer. In other embodiments, the base is present in an amount sufficient to provide from about 0.3 equivalents to about 0.6 equivalents of base, for example about 0.3 equivalents, about 0.35 equivalents, about or 0.4 equivalents of base, about 0.45 equivalents of base, about 0.5 equivalents of base, about 0.55 equivalents of base, or about 0.6 equivalents of base per equivalent of carboxylate groups in the polymer. In some embodiments, the base is present in an amount sufficient to provide about 0.5 equivalents of base per equivalent of carboxylate groups in the polymer.

For oral administration, the disclosed polymers may be formulated in a composition, formulation and/or dosage form readily by combining them with pharmaceutically acceptable carriers well known in the art. Such carriers enable the compositions of the disclosure to be formulated, as tablets, chewable tablets, pills, dragees, capsules, liquids, gel packs, gel blocks, syrups, slurries, suspensions, wafers, sachets, powders, dissolving tablets and the like, for oral ingestion by a subject, including a subject to be treated. In some embodiments, the compositions have a coating. In some embodiments, the compositions or capsules containing the compositions have an enteric coating. In other embodiments, the compositions or capsules containing the compositions, do not have an enteric coating.

In some embodiments, a composition, formulation, and/or dosage form as described herein comprises a base and a crosslinked polycarboxylate polymer as described herein (e.g., a cross-linked polyacrylic acid polymer), wherein the polymer further comprises calcium cations, wherein the calcium cations are counterions to about 15% to about 35% of the carboxylic acid groups in the polymer (alternately, counterions to about 15% to about 30%, about 20% to about 30%, or about 25% to about 35% of the carboxylate groups in the polymer), and is administered in an amount sufficient to provide from about 0.01 moles of carboxylate groups to about 0.5 moles or about 1.4 moles of carboxylate groups to the subject per day, for example, about 0.01 moles, about 0.02 moles, about 0.03 moles, about 0.04 moles, about 0.05 moles, about 0.06 moles, about 0.07 moles, about 0.08 moles, about 0.09 moles, about 0.1 moles, about 0.11 moles, about 0.12 moles, about 0.13 moles, about 0.14 moles, about 0.15 moles, about 0.16 moles, about 0.17 moles, about 0.18 moles, about 0.19 moles, about 0.2 moles, about 0.21 moles, about 0.22 moles, about 0.23 moles, about 0.24 moles, about 0.25 moles, about 0.26 moles, about 0.27 moles, about 0.28 moles, about 0.29 moles, about 0.3 moles, about 0.31 moles, about 0.32 moles, about 0.33 moles, about 0.34 moles, about 0.35 moles, about 0.36 moles, about 0.37 moles, about 0.38 moles, about 0.39 moles, about 0.4 moles, about 0.41 moles, about 0.42 moles, about 0.43 moles, about 0.44 moles, about 0.45 moles, about 0.46 moles, about 0.47 moles, about 0.48 moles, about 0.49 moles, about 0.5 moles, about 0.6 moles, about 0.7 moles, about 0.8 moles, about 0.9 moles, about 1.0 moles, about 1.1 moles, about 1.2 moles, about 1.3 moles, or about 1.4 moles of carboxylate groups to the subject per day. In a preferred embodiment, the dosage forms are administered in an amount sufficient to provide from about 0.01 to about 0.25 moles of carboxylate groups per day. In a more preferred embodiment, the dosage forms are administered in an amount sufficient to provide from about 0.1 to about 0.25 moles of carboxylate groups per day.

In some embodiments, the dosage form comprises a base and a crosslinked polycarboxylate polymer as described herein, (e.g., a cross-linked polyacrylic acid polymer), wherein the polymer further comprises calcium and/or magnesium cations, wherein the calcium and/or magnesium cations are counterions to about 15% to about 35% of the carboxylic acid groups in the polymer (alternately, counterions to about 15% to about 30%, about 20% to about 30%, or about 25% to about 35% of the carboxylate groups in the polymer), and is administered in an amount sufficient to provide from about 1 g to about 30 g or about 1 g or up to about 100 g or more of polymer per day, for example, about 1 g per day, about 2 g per day, about 3 g per day, about 4 g per day, about 5 g per day, about 6 g per day, about 7 g per day, about 8 g per day, about 9 g per day, about 10 g per day, about 11 g per day, about 12 g per day, about 13 g per day, about 14 g per day, about 15 g per day, about 16 g per day, about 17 g per day, about 18 g per day, about 19 g per day, about 20 g per day, about 21 g per day, about 22 g per day, about 23 g per day, about 24 g per day, about 25 g per day, about 26 g per day, about 27 g per day, about 28 g per day, about 29 g per day, about 30 g per day, about 35 g per day, about 40 g per day, about 45 g per day, about 50 g per day, about 55 g per day, about 60 g per day, about 65 g per day, about 70 g per day, about 75 g per day, about 80 g per day, about 85 g per day, about 90 g per day, about 95 g per day, or about 100 g of polymer per day or more.

In some embodiments, the dosage form is a sachet and contains a polymer or polymer-containing composition according to the present disclosure in sufficient amount to provide from about 1 g to about 30 g of the polymer. For example, a sachet may contain a composition according to the present disclosure in sufficient amount to provide about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 10.5 g, about 11 g, about 11.5 g, about 12 g, about 12.5 g, about 13 g, about 13.5 g, about 14 g, about 14.5 g, about 15 g, about 15.5 g, about 16 g, about 16.5 g, about 17 g, about 17.5 g, about 18 g, about 18.5 g, about 19 g, about 19.5 g, about 20 g, about 20.5 g, about 21 g, about 21.5 g, about 22 g, about 22.5 g, about 23 g, about 23.5 g, about 24 g, about 24.5 g, about 25 g, about 25.5 g, about 26 g, about 26.5 g, about 27 g, about 27.5 g, about 28 g, about 28.5 g, about 29 g, about 29.5 g, or about 30 g of polymer.

In some embodiments, the dosage form is a capsule containing an amount of a polymer or polymer-containing composition according to the present disclosure sufficient to provide from about 0.1 g to about 1 g of the polymer. For example, a capsule may contain an amount of a composition according to the present disclosure that is sufficient to provide about 0.1 g, about 0.15 g, about 0.2 g, about 0.25 g, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, or about 1 g of polymer.

In some embodiments, the dosage form is a tablet that contains an amount of a polymer or polymer-containing composition according to the present disclosure to provide from about 0.3 g to about 1 g of the polymer. For example, the tablet may contain about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, or about 1 g of polymer. In some embodiment, a disclosed composition is formulated as a tablet that is spherical or substantially spherical.

In some embodiments, the dosage form is a sachet, flavored bar, gel block, gel pack, pudding, or powder that contains an amount of a polymer or polymer-containing composition according to the present disclosure to provide from about 1 g to about 30 g of the polymer. For example, the sachet, flavored bar, gel block, gel pack, pudding, or powder may contain an amount of a composition according to the present disclosure to provide about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 0.19 g, about 20 g, about 21 g, about 22 g, about 23 g, about 24 g, about 25 g, about 26 g, about 27 g, about 28 g, about 29 g, or about 30 g of the polymer.

In some embodiments, the dosage form is a suspension or an oral suspension that contains an amount of a polymer or polymer-containing composition according to the present disclosure to provide from about 1 g to about 30 g of the polymer. For example, the suspension or oral suspension may contain an amount of a composition according to the present disclosure to provide about 2 g, about 3 g, about 4 g, about 5 g, about 6 g, about 7 g, about 8 g, about 9 g, about 10 g, about 11 g, about 12 g, about 13 g, about 14 g, about 15 g, about 16 g, about 17 g, about 18 g, about 19 g, about 20 g, about 21 g, about 22 g, about 23 g, about 24 g, about 25 g, about 26 g, about 27 g, about 28 g, about 29 g, or about 30 g of the polymer.

In some embodiments, compositions, formulations, and/or dosage forms according to the present disclosure further include an additional agent. In related embodiments, the additional agent is one that causes, routinely causes, typically causes, is known to cause, or is suspected of causing an increase in an ion level in at least some subjects upon administration. For example and without limitation, the additional agent may be an agent known to cause an increase in serum potassium levels in at least some subjects upon administration. For example and without limitation, the additional agent may be an agent known to cause an increase in serum sodium levels in at least some subjects upon administration. In related embodiments, the additional agent may be one or more of: a tertiary amine, spironolactone, fluoxetine, pyridinium and its derivatives, metoprolol, quinine, loperamide, chlorpheniramine, chlorpromazine, ephedrine, amitryptyline, imipramine, loxapine, cinnarizine, amiodarone, nortriptyline, a mineralocorticosteroid, propofol, digitalis, fluoride, succinylcholine, eplerenone, an alpha-adrenergic agonist, a RAAS inhibitor, an ACE inhibitor, an angiotensin II receptor blocker, a beta blocker, an aldosterone antagonist, benazepril, captopril, enalapril, fosinopril, lisinopril, moexipril, perindopril, quinapril, ramipril, trandolapril, candesartan, eprosartan, irbesartan, losartan, valsartan, telmisartan, acebutolol, atenolol, betaxolol, bisoprolol, carteolol, nadolol, propranolol, sotalol, timolol, canrenone, aliskiren, aldosterone synthesis inhibitors, VAP antagonists, amiloride, triamterine, a potassium supplement, heparin, a low molecular weight heparin, a non-steroidal anti-inflammatory drug, ketoconazole, trimethoprim, pentamide, a potassium sparing diuretic, amiloride, and/or triamterene. In some embodiments, the additional agent may cause fluid retention and/or maldistribution in at least some subjects upon administration.

The polymer, compositions, formulations, and/or dosage forms of the present disclosure may be administered in combination with other therapeutic agents. The choice of therapeutic agents that may be co-administered with the compositions of the disclosure will depend, in part, on the condition being treated.

Polymers, compositions, formulations, and/or dosage forms of the present disclosure may be administered in combination with a therapeutic agent that causes an increase, or is known to commonly cause an increase, in one or more ions in the subject. By way of example only, the crosslinked cation-binding polymer of the present disclosure may be administered with a therapeutic agent that causes an increase, or is known to commonly cause an increase, in the potassium and/or sodium level of a subject.

### Therapeutic Uses

The disclosed polymers, and compositions, formulations, and/or dosage forms comprising the disclosed polymers may be useful for therapeutic use, including to treat a subject with a disease and/or disorder, for example, to ameliorate, alleviate, or eliminate at least one symptom of the disease or disorder. Additionally or alternatively, the disclosed polymers, compositions comprising the disclosed polymers and/or dosage forms comprising the disclosed polymers may be used prophylactically to prevent a subject from becoming afflicted with a disease and/or disorder. In any of the methods of treatment or prophylaxis described herein, a base may be co-administered along with the polymer, or composition, formulation and/or dosage form comprising the polymer, either simultaneously or sequentially. The base may be included in the same composition, formulation, or dosage form or alternatively may be administered separately from the polymer, or composition, formulation, or dosage form containing the polymer, for example in a separate composition, formulation, or dosage form which is co-administered at the same time or before or after the polymer or composition, formulation, or dosage form that contains the polymer.

In some embodiments, polymers as disclosed herein, and/or compositions, formulations, and/or dosage forms containing the polymers, may be used in methods to treat or prevent fluid accumulation and/or maldistribution, and/or ion (e.g., sodium and/or potassium) accumulation and/or imbalances.

Many medical diseases and disorders may either result from, may cause, or may be associated with imbalances of total body fluid, local fluid accumulation in certain tissues or organs, total body ion stores, intracellular ion stores, serum ion levels, or extracellular ion stores. Ions involved in these imbalances may include sodium, potassium, magnesium, hydrogen, ammonium, chloride, bicarbonate, phosphate, and/or calcium. Various combinations of these imbalances are common. Some diseases, disorders, and states may result in excessive accumulations of potassium, sodium, and/or fluid, in various combinations of these overloads and with overloads sometimes occurring either as total body overload or as localized areas of excessive accumulation.

Fluid imbalances may sometimes result in too little fluid in the body (e.g., dehydration), too much fluid in the body (e.g., fluid overload), localized fluid accumulations, or combinations of these. For example, chronic malabsorptive diarrhea may sometimes result in total body dehydration accompanied by protein-calorie malnutrition with resultant edema (localized fluid overload) of the extremities and/or ascites. Metabolic processes associated with this state may result in excessive sodium stores in the body and depletion of total body potassium. Progression of the pathological mechanisms may also occur as malnutrition increases, and less protein may be available for tissue repair. The lining of the gastrointestinal tract can be sensitive to such a progression, as lack of protein and energy can inhibit the normal rapid turnover of villi, which may result in blunted villus architecture and further inhibition of protein absorption. Attempts to treat the disease state may sometimes exacerbate the progression of the disease. Unless intervention occurs early in the process, full provision of even the normal minimal daily requirements of nutrients may sometimes result in sudden death, possibly due to significant shifts in potassium, hydrogen, sodium, and/or calcium levels in the subject. Fluid removal using diuretics may sometimes cause sudden death, possibly from potassium loss associated with loop diuretics or thiazide diuretics.

Hepatic cirrhosis may sometimes result from various infections (e.g., hepatitis A, hepatitis B, schistomiasis, etc.), various toxins (e.g., ethanol, chloroform, isoflurane, etc), reactions to various drugs (e.g., chronic iron overload, methotrexate, terbinafine, nicotinic acid, acetaminophen, phenothiazines, etc.), immune diseases (e.g., primary biliary cirrhosis, primary sclerosing cholangitis, autoimmune hepatitis, etc.), and various genetic disorders (e.g., Wilson's disease, hemochromatosis, etc.) or congenital abnormalities (e.g., biliary atresia, etc.). Hepatic cirrhosis can restrict blood return from intraperitoneal organs to the heart and simultaneously decreases hepatic synthesis of albumin. Other hepatic diseases, such as hepatocarcinoma, infiltrative diseases, and storage diseases also may sometimes result in compromised venous return to the heart, though albumin synthesis may sometimes remain intact for much longer times. This combination of increased mechanical pressure in the intraperitoneal venous system accompanied by decreased intravascular oncotic pressure can result in ascites while also restricting the systemic blood supply. Decreased systemic vascular volume can decrease renal perfusion, which can trigger fluid and sodium retention, sometimes causing total body sodium overload. Cirrhosis can be progressive with gradually increasing hepatic fibrosis, gradually increasing venous pressure in the intraperitoneal organs, and gradually decreasing oncotic pressure. Thus, early in the disease, loop or thiazide diuretics may have some effect, but increase the risk of both hyponatremia and hypokalemia. The use of aldosterone antagonists such as spironolactone or eplerinone is common, but generally carries a risk of hyperkalemia. Amiloride can be used to remove fluid, but may increase the risk of hyperkalemia and the risk of hypotension. Increasing ascites can result in pressure on intra abdominal organs which can sometimes cause tissue damage as the disease worsens. The ascetic pressure can also restrict the flow of venous blood through the inferior vena cava resulting in pedal edema. Removal of the fluid through repeated paracenteses or through placement of a shunt from the peritoneal space to the superior vena cava, or from the portal vein to the superior vena cava, may become necessary. As the intraperitoneal venous pressure increases, porto-systemic shunts develop to as esophageal varices and hemorrhoidal varices open and widen. These may relieve portal pressure, but worsen hypoalbuminemia, rupture and bleed, and flood the systemic circulation with ammonium ions at toxic levels.

Heart failure may be defined as failure of the heart to adequately pump blood throughout the circulation. There are several classification methods for heart failure. One such classification method is to describe the particular portion of the pumping that is the major site of poor pump performance, but since the pump is moving blood through a closed, circular system, pump failure at any one point affects the flow through the rest of the system. Using this method of classification, heart failure can result from compromised movement of blood into the right atrium (heart failure with backwards failure from the right heart) which may result from conditions including pericardial effusion, pericardial tampanade, tricuspid valvular stenosis, tricuspid valvular insufficiency, or myocardial infiltrative diseases. Heart failure due to right heart backwards failure may result in increased systemic venous pressure and ensuing peripheral edema. Simultaneously, the poor filling of the right atrium may result in poor systemic arterial perfusion, due to low fluid output from the left heart, which may result in poor renal perfusion, reabsorption of sodium and fluid in the renal tubules, and total body fluid and sodium overload even though intravascular fluid volume is low. Similarly, heart failure with forward failure of the right heart may produce reduced ejection into the pulmonary artery with resultant increased pressure opposing complete emptying of the right atrium. This may result from diseases including, for example, pulmonary valvular stenosis, cor pulmonale, or pulmonary fibrosis. Symptoms and abnormalities of fluid and sodium accumulations can resemble those of right heart backward failure. Left heart backward failure may leave extra blood in the pulmonary circulation causing problems such as increased pulmonary venous pressure, pulmonary edema, poor systemic arterial perfusion with resultant renal retention of fluid and sodium. Causes of left heart backwards failure may include, for example, hypertrophic cardiomyopathy, myocardial infiltrative diseases, hypertensive heart disease, mitral stenosis, and mitral insufficiency. Left heart forward failure may result when there is a reduced ejection of blood into the aorta resulting in poor systemic perfusion that can cause renal retention of fluid and sodium and organ damage which can result in metabolic and oxidative stress. Extreme degrees of left heart forward failure frequently may result in left heart backward failure which may be due to diminished space to accept blood from the left atrium. Myocardial infarction with fibrotic replacement of myocardium is the most to common cause of left heart forward failure, but other causes may include, for example, dilated cardiomyopathy, persistent arrhythmias, and aortic valvular stenosis.

Alternatively, heart failure may be classified as systolic heart failure or diastolic heart failure. Generally, systolic heart failure may be associated with the incomplete emptying of the left ventricle which then becomes dilated, leading to worsening of the emptying of the ventricle during contraction. It may be diagnosed by the finding on echocardiography of a low left ventricular ejection fraction, which may result from left ventricular dilatation rather than a fall in the actual amount of blood being ejected from the heart with each contraction. This ventricular dilatation feeds backward and may result in excessive blood in the pulmonary vascular system, pulmonary edema, and possible transudation of fluid from the pulmonary venous system into interstitial spaces, pleural space, and pericardial space. This excessive fluid can cause rales, progressing to orthopnea, progressing to paroxysmal nocturnal dyspnea, and progressing to poor exchange of gases in the lungs with resultant hypoxia and hypercapnea

Stages of heart disease are currently based on the degree of accumulated pulmonary fluid and the associated effects on the patient's ability to breathe. New York Heart Association (NYHA) Class I heart failure may be defined as heart failure where there is no limitation of physical activity and there is no undue fatigue, palpitation or shortness of breath with normal physical activity. NYHA Class II heart failure occurs when there is slight limitation of ordinary physical activity because of fatigue, palpitation, or dyspnea whenever the person is not at rest. NYHA Class III heart failure occurs when the person is comfortable at rest but even less than ordinary physical activity causes fatigue, palpitation, or dyspnea. NYHA Class IV heart failure occurs when the person is not able to carry out any physical activity without discomfort from fatigue, palpitations, or dyspnea, and these symptoms are even present at rest. When awareness of the importance of dietary sodium restriction increased, and when diuresis became available, it became possible to control the dyspnea. The disease was then more commonly referred to as “heart failure” rather than “congestive heart failure.” This allowed the recognition of the second member of this classification system: diastolic heart failure (also called normal ejection fraction heart failure). Diastolic heart failure generally has less ventricular dilatation than systolic heart failure and, therefore, a lower end diastolic volume for use in the denominator of the calculation of ejection fraction. Symptoms may include, for example, fatigue, poor exercise tolerance, and excessive energy expenditure by the heart. However, diastolic heart failure can progress to remodeling of the ventricular architecture with dilatation, hypertrophy, and/or myocyte loss, resulting in systolic heart failure.

Heart failure is a progressive disease. Myocytes can be damaged by increased pressure and dilation of the heart. As pre-load increases, myocytes may be unable to relax completely. As afterload increases, more energy may be required for each contraction. Myocytes may die as a result of this excessive demand, and the replacement of the myocytes eventually cannot keep pace with the death rate. Remodeling of both the size of the ventricle and the wall thickness occurs with both myocytes and fibrous tissue. As the disease progresses, the adrenergic cardiac nervous system responds with excessive release of norepinephrine to improve the ability of the myocyte to contract (improve myocardial contractility). The renin-angiotensin-aldosterone system (RAAS) may be activated to increase renal reabsorption of fluid in an attempt to maintain arterial pressure so that tissue perfusion can remain normal. This may result in more fluid than sodium retention and may lead to hyponatremia even though total body sodium is elevated. Vasopressin, epinephrine, and endothelin-1 increase, causing vasoconstriction, which can sometimes support systemic pressure. If successful, this increase in systemic pressure increases afterload on the myocytes of the left ventricle, increasing calcium levels in the myocytes via increased cyclic AMP. The enhanced calcium entry into the myocytes improves myocyte contractility but impairs their relaxation (i.e., improved inotropy but impaired lusitropy). This contributes to increased myocyte death and cardiac fibrosis. In addition, the increased myocyte intracellular calcium concentrations can enhance the development of cardiac arrhythmias. Even without arrhythmias, cardiac energy expenditure increases as a result of these neurohormonal changes causing increased fatigue, myocyte fatigue, and eventual inability of the neurohormonal changes to sustain the systemic pressure.

Since heart failure is a progressive disease, treatment options at various stages may differ, and may have undesirable side effects on later stages of the disease. Although traditional treatment with loop or thiazide diuretics can counteract the water and sodium retention, at least until more advanced heart failure is present, problems with hypokalemia can occur, which may exacerbate the arrhythmias that may result from myocytes overloaded with intracellular calcium. However, these treatments have little effect on the progression of cardiac fibrosis. Addition to or replacement of these traditional diuretics with agents designed to inhibit the fluid and sodium retention related to the RAAS system can benefit heart failure patients as they not only serve to decrease the body fluid and sodium overload, but also are protective against myocyte damage and cardiac fibrosis. Such agents may include, for example, angiotensin converting enzyme inhibitors (ACE inhibitors) such as captopril, lisinopril, or ramipril; and angiotensin receptor blockers (ARBs) such as losartan, valsartan, telmisartan, eprosartan, or candesartan. RAAS inhibitors may also include aldosterone antagonists such as spironolactone and eplerenone. However, these agents may increase serum potassium, frequently to the point of causing hyperkalemia. Hyperkalemia also increases the risk of arrhythmias and sudden death.

Beta adrenergic receptor blockers (“beta blockers”) have also been shown to improve survival in heat failure patients. By interrupting the increased adrenergic input, these agents reduce the myocyte contractility, allowing them to return to a more physiological state with better relaxation in diastole, less intracellular calcium signaling, slower heart rate, and diminished death rate of myocytes. This decreases, and may even reverse, cardiac remodeling and may return ventricular size to normal. Beta blockers may include, for example, metoprolol, carvedilol, and bisprolol. However, because these beta blockers partially block renin release, they can also increase serum potassium. The increase in serum potassium associated with the use of ACE inhibitors, ARBs, aldosterone inhibitors, and beta blockers may prevent the optimal treatment of patients with these agents. Other medications, such as inotropic agents, vasodilators, and human B-type natriuretic peptides may also be used in various stages of the progression of heart failure, but may be less effective at balancing fluid, sodium, and potassium.

Chronic kidney disease and its progression to End Stage Renal Disease (ESRD) may compromise the ability of the kidney to excrete fluid, potassium, sodium, and many other metabolic wastes. Chronic kidney disease can be caused by many different conditions. These may include, for example: (1) congenital anomalies such as hypoplastic kidney and renal arterial malformations, (2) genetic abnormalities such as polycystic kidney disease, Potter's syndrome, and prune belly syndrome, (3) infectious and immune diseases such as endocarditis, post-streptococcal glomerulonephritis, IgA nephropathy, lupus erythematosis nephritis, anti-glomerular basement membrane disease, E. coli Shiga toxin, and focal segmental glomerulosclerosis, (4) damage by toxins such as hydrocarbon solvent exposure, anti-neoplastic medications, anti-fungal medications such as Amphotericin B, and heavy metal exposure, (5) hypertension, and (6) diabetes. Regardless of the cause of the chronic kidney disease, the impaired ability of the kidney to excrete fluid, sodium, and potassium results in imbalances of these substances within the body.

Chronic kidney disease (CKD) may be graded by the creatinine clearance through the kidney measured in milliliters of blood cleared of creatinine per minute, or graded by the glomerular filtration rate (GFR) in milliliters of fluid filtered through the kidney per minute corrected for the size of the person as determined by body surface area. The normal GFR may be above 90 mL/min/1.73 m² (e.g., 90 milliliters of fluid filtered per minute per 1.73 square meters of body surface area). CKD 1 may be present when there is evidence of kidney damage but the GFR remains above 90 mL/min/1.73 m². CKD 2 may be defined as the presence of kidney disease with GFR between 60 and 89 mL/min/1.73 m². CKD 3 may be present when the GFR is between 30 and 59 mL/min/1.73 m². CKD 4 (severe chronic kidney disease) may be present when the GFR is between 15 and 29 mL/min/1.73 m². CKD 5 is also called ESRD and is present when the GFR is below 15 mL/min/1.73 m². In the early stages of chronic kidney disease, fluid and sodium may be retained in the body due to low filtration. Treatment may include the use of traditional loop or thiazide diuretics. This may result in the same changes described above for heart failure with potassium wasting and the risk of hypokalemia, the risk of hyponatremia, and eventual diuretic failure. RAAS inhibitors may sometimes be administered, but their use may be limited by the potential to induce hyperkalemia. Once the final progression to ESRD is completed, no clinically significant fluid, sodium, potassium, or other metabolic byproducts can be excreted. Patients must be placed on either hemodialysis or peritoneal dialysis to survive. However, both hemodialysis and peritoneal dialysis are imperfect in their removal of fluid and Sodium. Patients must restrict their fluid intake to approximately 1000 milliliters of water daily from all sources in order for hemodialysis to adequately and safely return the total body water to a safe level. Patients must restrict their sodium intake to 1500 mg or less to be able to maintain safe blood pressure and allow the dialysis to adequately remove the ingested sodium. However, these restrictions are quite onerous. Frequently, patients fail to abide by these restrictions, resulting in severe symptoms and possible health risks—including death—to the patient. Even if a patient is compliant, attempts to remove large volumes of fluid during a hemodialysis session can cause intradialytic hypotension with symptoms including cramps, dizziness, fainting, and temporary blindness, and can result in strokes, intestinal infarction, myocardial infarctions, and even death.

Hypertension is a condition that may be characterized by an increased pressure within the vascular system. Different causes of hypertension are known. Salt-sensitive hypertension may result, usually after prolonged high dietary sodium intake, when the kidney reabsorbs an excessive amount of sodium from the glomerular filtrate. Arterial stenosis, particularly renal artery stenosis, may result in hypertension. Endocrine abnormalities may result in excessive corticosteroid or antidiuretic hormone production cause hypertension. Genetic influences that may cause hypertension are known to be present, but are poorly understood. Regardless of the cause of hypertension, the renal perfusion may decrease in an attempt by the macula densa to protect the glomerulus from excessive pressure. The decreased renal perfusion may result in accumulation of fluid and sodium in the body. This fluid and sodium accumulation initially may be perivascular and in interstitial spaces, resulting in minimal early symptoms. At this stage, the Dietary Approaches to Stop Hypertension study (F. M. Sachs, et al., New England Journal of Medicine, vol. 344, pp. 3-10 (2001)) suggests that there may be a sodium overload and a potassium deficiency with fluid balance being less important. However, sodium overload eventually may cause fluid retention and fluid overload. As intravascular fluid volume increases, the left heart may begin to respond to increased pressure with development of heart failure with attendant ventricular enlargement, myocyte changes, neurohormonal alterations, and cardiac fibrosis. Early treatment of hypertension with traditional diuretics, beta blockers, ACE inhibitors, ARBs, or aldosterone inhibitors can prevent this progression, but the diuretics are viewed by patients as unpleasant due to increased urination and urgent urination. RAAS inhibitors and beta blockers may cause cough, dry mucus membranes, male gynecomastia, slow heart rate, and sexual dysfunction. Hyperkalemia from the use of RAAS inhibitors may limit their use, resulting in inadequate therapy. Most patients require multiple agents from the list to control the hypertension, therefore most patients experience side effects. Thus, compliance with available treatment options for hypertension is quite low.

Other diseases and conditions associated with abnormal balances of fluid, sodium, and/or potassium may include hyperkalemia, which may result from excessive release of intracellular potassium as the result of acidosis, diabetic ketoacidosis, thermal burns, electrical burns, hemolysis, gastrointestinal bleeding, crush injuries, tumor response to chemotherapeutic treatment, ingestion of potassium, adrenal insufficiency, and rhabdomyolysis. Hypernatremia, the elevation of the serum sodium level, is rare and usually occurs only with severe restriction of fluid intake, excessive loss of hypotonic fluid (such as the syndrome of inappropriate antidiuretic syndrome or heatstroke), or excessive sodium intake. In both severe fluid intake restriction and excessive hypotonic fluid excretion, the total body stores of sodium may be normal or even low in spite of the elevated serum levels. Total body sodium overload, however, is common to many of the diseases and conditions already mentioned as well as other diseases causing edema such as inflammatory bowel disease. Fluid overload and localized fluid accumulations or maldistribution may be present in such conditions as premenstrual syndrome, chronic venous insufficiency, angioneurotic edema, allergic edema, and lymphedema. Treatments for many of these diseases and conditions are the same as the therapies mentioned above for removing fluid, potassium, and sodium from a patient.

As is evident from this disclosure, there is need for a more effective and safer method to remove fluid, sodium, and/or potassium from patients with malabsorptive diarrhea, protein-calorie malnutrition, ascites, heart failure, chronic kidney disease, end stage renal disease, hypertension, edema, hyperkalemia, and other disorders of fluid and electrolyte metabolism. In many cases, the need for removal of these substances differs at different stages of progression of a disease. Early disease may require removal of primarily fluid or of fluid and sodium while more advanced disease or the use of ACE inhibitors, ARBs, aldosterone antagonists, or beta blockers may require removal of larger amounts of potassium alone or of potassium and fluid. At some stages, removal of all three substances may be needed. However, the medications require treatment with combinations of medications and have disruptive side effects that make it desirable to have a new medication for control of these substances.

Surprisingly, the polymers of the present disclosure, and compositions, formulations, and dosage forms of the present disclosure that comprise crosslinked cation-binding polymers comprising monomers containing carboxylic acid groups (e.g., polyacrylate polymers) and wherein the polymer further comprises calcium and/or magnesium cations, wherein the calcium and/or magnesium cations are counterions to about 15% to about 35% of the carboxylate groups in the polymer (alternatively, counterions to about 15% to about 30%, about 20% to about 30%, or about 25% to about 35% of the carboxylate groups in the polymer, or any of about 15%, about 20%, about 25%, about 30%, or about 35% of the carboxylate groups in the polymer), and wherein sodium cations (if present) are counterions to no more than about 5% of the carboxylate groups in the polymer, are optimized for maintaining the cation binding and/or removal properties of the polymer (e.g., for potassium and sodium) and/or the fluid binding and/or removal properties of the polymer in humans, while, in some embodiments, minimizing or eliminating changes in acid/base balance in an individual to whom the composition, formulation, or dosage form is administered. As such, the polymers and compositions, formulations, and/or dosage forms containing the polymers as described herein are useful for the treatment of a variety of diseases or disorders, including those involving ion (e.g., potassium and/or sodium) and/or fluid imbalances (e.g., overloads).

The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers) and wherein the polymer further comprises calcium and/or magnesium cations, wherein the calcium and/or magnesium cations are counterions to about 15% to about 35% of the carboxylate groups in the polymer (alternatively, counterions to about 15% to about 30%, about 20% to about 30%, or about 25% to about 35% of the carboxylate groups in the polymer, or any of about 15%, about 20%, about 25%, about 30%, or about 35% of the carboxylate groups in the polymer), and wherein sodium cations (if present) are counterions to no more than about 5% of the carboxylate groups in the polymer, may be used in methods for the removal of fluid and/or ions (e.g., potassium and/or sodium) from a subject, i.e., The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers), and wherein the polymer further comprises calcium and/or magnesium cations, wherein the calcium and/or magnesium cations are counterions to about 15% to about 35% of the carboxylate groups in the polymer (alternatively, counterions to about 15% to about 30%, about 20% to about 30%, or about 25% to about 35% of the carboxylate groups in the polymer, or any of about 15%, about 20%, about 25%, about 30%, or about 35% of the carboxylate groups in the polymer), and wherein sodium cations (if present) are counterions to no more than about 5% of the carboxylate groups in the polymer, may be used in methods for the removal of fluid from a subject.

The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers), and wherein the polymer further comprises calcium and/or magnesium cations, wherein the calcium and/or magnesium cations are counterions to about 15% to about 35% of the carboxylate groups in the polymer (alternatively, counterions to about 15% to about 30%, about 20% to about 30%, or about 25% to about 35% of the carboxylate groups in the polymer, or any of about 15%, about 20%, about 25%, about 30%, or about 35% of the carboxylate groups in the polymer), and wherein sodium cations (if present) are counterions to no more than about 5% of the carboxylate groups in the polymer, may also be used in methods for treating diseases or disorders associated with increased retention of fluid and/or ion imbalances.

The disclosed polymers, compositions comprising the disclosed polymers, formulations comprising the disclosed polymers, and/or dosage forms comprising the disclosed polymers which comprise repeat units containing carboxylic acid groups, (e.g., polyacrylate polymers) and wherein the polymer further comprises calcium and/or magnesium cations, wherein the calcium and/or magnesium cations are counterions to about 15% to about 35% of the carboxylate groups in the polymer (alternatively, counterions to about 15% to about 30%, about 20% to about 30%, or about 25% to about 35% of the carboxylate groups in the polymer, or any of about 15%, about 20%, about 25%, about 30%, or about 35% of the carboxylate groups in the polymer), and wherein sodium cations (if present) are counterions to no more than about 5% of the carboxylate groups in the polymer, may also be used in methods to treat end stage renal disease (ESRD), chronic kidney disease (CKD), congestive heart failure (CHF), hyperkalemia, hypernatremia, or hypertension.

The disclosed polymers, compositions comprising the disclosed polymers, formulations comprising the disclosed polymers, and/or dosage forms comprising the disclosed polymers which comprise repeat units containing carboxylic acid groups (e.g., polyacrylate polymers), and wherein the polymer further comprises calcium and/or magnesium cations, wherein the calcium and/or magnesium cations are counterions to about 15% to about 35% of the carboxylate groups in the polymer (alternatively, counterions to about 15% to about 30%, about 20% to about 30%, or about 25% to about 35% of the carboxylate groups in the polymer, or any of about 15%, about 20%, about 25%, about 30%, or about 35% of the carboxylate groups in the polymer), and wherein sodium cations (if present) are counterions to no more than about 5% of the carboxylate groups in the polymer, may be used to remove one or more ions selected from the group consisting of: sodium, potassium, calcium, magnesium, iron, and/or ammonium.

In some embodiments, the polymers, compositions comprising the disclosed polymers, and/or dosage forms comprising the disclosed polymers as disclosed herein may be substantially coated with a coating, e.g., an enteric coating, that allows it to pass through the gut, e.g., upper gastrointestinal tract, and open in the intestine where the polymer may absorb fluid and/or specific ions that are concentrated in that particular portion of the intestine. In other embodiments, the polymers, compositions comprising the disclosed polymers, and/or dosage forms comprising the disclosed polymers disclosed herein do not comprise such a coating. In some embodiments, the absorbent material, i.e., polymer as disclosed herein, may be encapsulated in a capsule. In one embodiment, the capsule may be substantially coated with a coating, e.g., an enteric coating, that allows it to pass through the gut and open in the intestine where the capsule may release the polymer to absorb fluid or specific ions that are concentrated in that particular position of the intestine. In another embodiment, the capsule does not contain such a coating. Individual particles of polymer or groups of particles may be encapsulated or alternatively, larger quantities of beads or particles may be encapsulated together.

In some embodiments, polymers as disclosed herein may be milled to give finer particles in order to increase drug loading of capsules, or to provide better palatability for formulations such as gels, bars, puddings, or sachets. In addition, milled particles or groups of particles, or unmilled polymeric material (e.g., beads) may be coated with various common pharmaceutical coatings. These coatings may or may not have enteric properties but will have the common characteristic that they will separate the polymer from the tissues of the mouth and prevent the polymer from adhering to tissue. For example, such coatings may include, but are not limited to: a single polymer or mixtures thereof, such as may be selected from polymers of ethyl cellulose, polyvinyl acetate, cellulose acetate, polymers such as cellulose phthalate, acrylic based polymers and copolymers or any combination of soluble, insoluble polymers or polymer systems, waxes and wax based coating systems.

In some embodiments, the polymers disclosed herein for administration to an individual or inclusion in a composition, formulation, or dosage form for administration to an individual, e.g., for use in a method of treatment as disclosed herein, are individual particles or particles agglomerated to form a larger particle (for example, flocculated particles), and have a diameter of about 1 to about 10,000 microns (alternatively, about 1 micron to about 50 microns, about 10 microns to about 50 microns, about 10 microns to about 200 microns, about 50 microns to about 100 microns, about 50 microns to about 200 microns, about 50 microns to about 1000 microns, about 500 microns to about 1000 microns, about 1000 to about 5000 microns, or about 5000 microns to about 10,000 microns). In some embodiments, the particles or agglomerated particles have a diameter of about 1, about 5, about 10, about 20, about 30, about 40, about 50, about 60, about 70, about 80, about 90, about 100, about 110, about 120, about 130, about 140, about 150, about 160, about 170, about 180, about 190, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 7000, about 7500, about 8000, about 8500, about 9000, about 9500, or about 10,000 microns. In one embodiment, the particles with a diameter of about 1 micron to about 10 microns.

In certain exemplary embodiments, the crosslinked cation-binding polymer, as described, for example, for administration to an individual or inclusion in a composition, formulation, or dosage form for administration to an individual, e.g., for use in a method of treatment as disclosed herein, is a crosslinked polyacrylate polymer (i.e., derived from acrylic acid monomers or a salt thereof) that comprises calcium and/or magnesium cations, wherein the calcium and/or magnesium cations are counterions to about 15% to about 35% of the carboxylate groups in the polymer, and wherein sodium cations (if present) are counterions to no more than about 5% of the carboxylate groups in the polymer. For example, the polymer may be a polyacrylate polymer crosslinked with about 0.08 mol % to about 0.2 mol % crosslinker, and for example, may comprise an in vitro saline absorption capacity of at least about 20 times its weight (e.g., at least about 20 grams of saline per gram of polymer, or “g/g”), at least about at least about 30 times its weight, at least about 40 times its weight, at least about 50 times its weight, at least about 60 times its weight, at least about 70 times its weight, at least about 80 times its weight, at least about 90 times its weight, at least about 100 times its weight, or more. In some embodiments, the crosslinked polyacrylate polymer comprises individual particles or particles that are agglomerated (for example, flocculated) to form a larger particle, wherein the individual or agglomerated particle diameter is about 1 to about 10,000 microns (alternatively, about 1 micron to about 10 microns, about 1 micron to about 50 microns, about 10 microns to about 50 microns, about 10 microns to about 200 microns, about 50 microns to about 100 microns, about 50 microns to about 200 microns, about 50 microns to about 1000 microns, about 500 microns to about 1000 microns, about 1000 to about 5000 microns, or about 5000 microns to about 10,000 microns. In some embodiments, administration of such a crosslinked polyacrylate polymer, comprising comprises calcium and/or magnesium cations, wherein the calcium and/or magnesium cations are counterions to about 15% to about 35% of the carboxylate groups in the polymer, does not change or does not significantly change acid/base balance in an individual to whom it is administered, for example, as measured by serum total bicarbonate, arterial blood pH, urine pH, and/or urine phosphorous. In some embodiments, a crosslinked polyacrylate polymer, comprising comprises calcium and/or magnesium cations, wherein the calcium and/or magnesium cations are counterions to about 15% to about 35% of the carboxylate groups in the polymer, is administered with an added base (e.g., up to about 0.8 equivalents of added base per equivalents of carboxylate groups in the polymer), and such administration of the polymer and base does not change or does not significantly change acid/base balance in an individual to whom it is administered, for example, as measured by serum total bicarbonate, arterial blood pH, urine pH, and/or urine phosphorous. In some embodiments, such a crosslinked polyacrylate polymer, comprising calcium and/or magnesium cations, wherein the calcium and/or magnesium cations are counterions to about 15% to about 35% of the carboxylate groups in the polymer, may be administered, optionally with added base as described herein, to an individual for removal of fluid and/or ions, for example, sodium and/or potassium cations, wherein such administration does not change or does not significantly change acid/base balance in the individual, for example, as measured by serum total bicarbonate, arterial blood pH, urine pH, and/or urine phosphorous.

In some embodiments, the polymer may be mixed with one or more base(s) in the same composition, formulation, and/or dosage form and may be in contact with fluid within the dosage from, such as suspensions or gels. To prevent interaction of the crosslinked cation-binding polymer and the base component before administration to a subject, pharmaceutical coatings known in the art can be used to coat the polymer, the base, or both to prevent or impede interaction of the polymer and the base. In some embodiments, the pharmaceutical coating may have enteric properties. As example, pharmaceutical coatings may include but are not limited to: a single polymeric coating or mixtures of more than one pharmaceutical coating, such as may be selected from polymers of ethyl cellulose, polyvinyl acetate, cellulose acetate; polymers such as cellulose phthalate, acrylic based polymers and copolymers, or any combination of soluble polymers, insoluble polymers and/or polymer systems, waxes and wax based coating systems. In alternate embodiments, the polymer and base are administered in separate dosage forms.

A subject (e.g., an individual or patient), as disclosed herein, includes a vertebrate, preferably a mammal, more preferably a human. Mammals include, but are not limited to, farm animals (such as cows), sport animals, pets (such as cats, dogs and horses), primates, and rodents (such as mice and rats). For purposes of treatment, prognosis and/or diagnosis, a subject includes any animal such as those classified as a mammal, including humans, domestic and farm animals, and zoo, wild, sports, or pet animals, such as dogs, horses, cats, cows, etc. Preferably, the subject for treatment, prognosis and/or diagnosis is human.

A disease or disorder includes any condition that would benefit from treatment with a composition as disclosed herein. This includes both chronic and acute diseases or disorders, including those pathological conditions which predispose the subject to the disease or disorder in question.

As used herein, treatment or treating refers to clinical intervention in an attempt to alter the natural course of the subject being treated, and can be performed either for prophylaxis (e.g., prevention) or during the course of clinical pathology (e.g., after the subject is identified as having a disease or disorder or the symptoms of a disease or disorder). Desirable effects of treatment include preventing occurrence or recurrence of disease, alleviation of symptoms, diminishment of any direct or indirect pathological consequences of the disease or disorder, decreasing the rate of disease progression, amelioration or palliation of the disorder, and remission or improved prognosis. Terms such as treating/treatment/to treat or alleviating/to alleviate refer to both 1) therapeutic measures that cure, slow down, lessen symptoms of, and/or halt progression of a diagnosed disease or disorder (e.g., a pathologic condition or disorder) and 2) prophylactic or preventative measures that prevent and/or slow the development of a disease or disorder (e.g., a targeted pathologic condition or disorder). Thus, those in need of treatment may include those already with the disease or disorder; those prone to have the disease or disorder; and those in whom the disease or disorder is to be prevented.

An effective amount refers to an amount effective, at dosages and for periods of time necessary, to achieve the desired therapeutic or prophylactic result. A therapeutically effective amount of a composition disclosed herein, may vary according to factors such as the disorder, age, sex, and weight of the subject, and the ability of the composition to elicit a desired response in the individual. A therapeutically effective amount is also one in which any toxic or detrimental effects of the composition are outweighed by the therapeutically beneficial effects. A prophylactically effective amount refers to an amount effective, at dosages and for periods of time necessary, to achieve the desired prophylactic result. Typically but not necessarily, since a prophylactic dose is used in subjects prior to or at an earlier stage of disease, the prophylactically effective amount may be less than the therapeutically effective amount. In some embodiments, a therapeutically effective amount includes administration of about 1 g to about 30 g or up to 100 g or more per day of a disclosed cross-linked polymer to an individual. In some embodiments, a prophylactically effective amount includes administration of about 1 g to about 30 g or up to 100 g or more per day of a disclosed cross-linked polymer to an individual. In various embodiments, base is co-administered at up to about 0.8 equivalents, for example, about 0.05 equivalents, about 0.1 equivalents, about 0.15 equivalents, about 0.2 equivalents, about 0.25 equivalents, about 0.3 equivalents, about 0.35 equivalents, about 0.4, equivalents, about 0.45 equivalents, about 0.5 equivalents, about 0.55 equivalents, about 0.6 equivalents, about 0.65 equivalents, about 0.7 equivalents, about 0.75 equivalents, or about 0.8 equivalents with respect to carboxylic acid groups on the polymer. A therapeutically or prophylactically effective amount of polymer and base may be administered in a single dosage or multiple doses, for example, administered once per day or administered 24 or more times daily, i.e., divided into and administered as 1, 2, 3, 4, or more doses per day, or administered at intervals of 2, 3, 4, 5, or 6 days, weekly, bi-weekly, etc.

Pharmaceutically acceptable includes approved or approvable by a regulatory agency of the Federal or a state government or listed in the U.S. Pharmacopeia or other generally recognized pharmacopeia for use in animals, including humans. A pharmaceutically acceptable salt includes a salt of a compound that is pharmaceutically acceptable and that possesses the desired pharmacological activity of the parent compound. A pharmaceutically acceptable excipient, carrier or adjuvant includes an excipient, carrier or adjuvant that can be administered to a subject, together with at least one composition of the present disclosure, and which does not destroy the pharmacological activity thereof and is nontoxic when administered in doses sufficient to deliver a therapeutic or prophylactic amount of the composition. A pharmaceutically acceptable vehicle includes a diluent, adjuvant, excipient, or carrier with which at least one composition of the present disclosure is administered.

Polymers, or compositions, formulations, and/or dosage forms comprising cross-linked cation binding polymers as disclosed herein can be used either alone or in combination with one or more other agents for administration to a subject (e.g., in a therapy or prophylaxis). As described herein, such combined therapies or prophylaxis include combined administration (where the polymer, composition, formulation, and/or dosage form and one or more agents are included in the same or separate composition, formulation, and/or dosage form) and separate administration, in which case, administration of the polymer, composition, formulation, and/or dosage form disclosed herein can occur prior to, contemporaneous with and/or following, administration of the one or more other agents (e.g., for adjunct therapy or intervention). Thus, co-administered or co-administration includes administration of the polymers, compositions, formulations, and/or dosage forms of the present disclosure before, during and/or after the administration of one or more additional agents or therapies.

In some embodiments, the disclosed polymers, compositions comprising the disclosed polymers, formulations comprising the disclosed polymers, and/or dosage forms comprising the disclosed polymers are useful for treating a disease or disorder. Typically, the disclosed polymers, compositions comprising the disclosed polymers, formulations comprising the disclosed polymer, and/or dosage forms comprising the disclosed polymers are co-administered with a base, as described herein. In some embodiments, the disease or disorder is one or more of: heart failure, a renal insufficiency disease, end stage renal disease, liver cirrhosis, chronic renal insufficiency, chronic kidney disease, fluid overload, fluid maldistribution, edema, pulmonary edema, peripheral edema, angioneurotic edema, lymphedema, nephrotic edema, idiopathic edema, ascites, cirrhotic ascites, chronic diarrhea, excessive interdialytic weight gain, high blood pressure, hyperkalemia, hypernatremia, abnormally high total body sodium, hypercalcemia, tumor lysis syndrome, head trauma, an adrenal disease, Addison's disease, salt-wasting congenital adrenal hyperplasia, hyporeninemic hypoaldosteronism, hypertension, salt-sensitive hypertension, refractory hypertension, hyperparathyroidism, renal tubular disease, rhabdomyolysis, electrical burns, thermal burns, crush injuries, renal failure, acute tubular necrosis, insulin insufficiency, hyperkalemic periodic paralysis, hemolysis, malignant hyperthermia, pulmonary edema secondary to cardiogenic pathophysiology, pulmonary edema with non-cardiogenic origin, drowning, acute glomerulonephritis, aspiration inhalation, neurogenic pulmonary edema, allergic pulmonary edema, high altitude sickness, Adult Respiratory Distress Syndrome, traumatic edema, cardiogenic edema, allergic edema, urticarial edema, acute hemorrhagic edema, papilledema, heatstroke edema, facial edema, eyelid edema, angioedema, cerebral edema, scleral edema, nephritis, nephrosis, nephrotic syndrome, glomerulonephritis, renal vein thrombosis, and/or premenstrual syndrome.

In some embodiments, the disclosed polymers, compositions comprising the disclosed polymers, formulations, comprising the disclosed polymers, and/or dosage forms comprising the disclosed polymers, as disclosed herein, are useful for treating a disease or disorder involving an ion imbalance in a subject by administering to the subject an effective amount of the polymer, composition, formulation, and/or a dosage form (e.g., an effective amount), as disclosed herein. In some embodiments, the polymers, compositions, formulations, and/or dosage forms may be co-administered with a base, as described herein. In some embodiments, the disease or disorder is or includes hyperkalemia. In some embodiments, the disease or disorder is or includes hypernatremia. In some embodiments, the disease or disorder is or includes an abnormally high total body sodium level. In some embodiments, the disease or disorder is or includes an abnormally high potassium level. In some embodiments, the disease or disorder is or includes hypernatremia and hyperkalemia. In some embodiments, the disease or disorder is or includes fluid overload. In some embodiments, the disease or disorder is or includes fluid overload and hyperkalemia. In some embodiments, the disease or disorder is or includes fluid overload and hyperkalemia and abnormally high total body sodium level.

In some embodiments, the disclosed polymers, compositions comprising the disclosed polymers, formulations comprising the disclosed polymers, and/or dosage forms comprising the disclosed polymers as disclosed herein are useful for treating a subject with heart failure by administering to the subject an effective amount of the polymer, composition, formulation, and/or dosage form as disclosed herein. In some embodiments, the polymers, compositions, formulations, and/or dosage forms may be co-administered with a base, as described herein. In some embodiments, the subject has both heart failure and chronic kidney disease. In some related embodiments, the methods further comprise reducing one or more symptoms of a fluid overload state in the subject. Symptoms of a fluid overload state in a subject are known to those skilled in the art, and may include, for example and without limitation, difficulty breathing when lying down, ascites, fatigue, shortness of breath, difficulty breathing on exertion, increased body weight, peripheral edema, and/or pulmonary edema. In some related embodiments, the subject may be on concomitant dialysis therapy. In some further related embodiments, the dialysis therapy may be reduced or discontinued after administration of a disclosed polymer, a composition comprising the disclosed polymer, a formulation comprising the disclosed polymer, and/or a dosage form comprising the disclosed polymer, as disclosed herein. In some related embodiments, the method further comprises identifying the subject as having heart failure before administering the disclosed polymer, composition comprising the disclosed polymer, formulation comprising a disclosed polymer, and/or dosage form comprising the disclosed polymer. In some embodiments, administration of the disclosed polymers, compositions comprising the disclosed polymers, and/or dosage forms comprising the disclosed polymers, as described herein, improves or ameliorates at least one symptom of heart failure, for example, at least one symptom that impacts the subject's quality of life and/or physical function. For example, administration may result in body weight reduction, dyspnea improvement (for example, overall and dyspnea at exertion), six minute walk test improvement, and/or improvement or absence of peripheral edema. In some embodiments, administration of the disclosed polymers, compositions comprising the disclosed polymers, and/or dosage forms comprising the disclosed polymers, as described herein, results in reduction of patient classification by at least one heart failure class, according to the New York Heart Association Class I, II, III, IV functional classification system

In some embodiments, the disclosed polymers, compositions comprising the disclosed polymers, formulations comprising the disclosed polymers, and/or dosage forms comprising the disclosed polymers, as disclosed herein, are useful for treating a subject with end stage renal disease (ESRD) by administering to the subject an effective amount of the polymer, composition, formulation, and/or dosage form, as disclosed herein. In some embodiments, the polymers, compositions, formulations, and/or dosage forms may be co-administered with a base, as described herein. In some related embodiments, the subject is on concomitant dialysis therapy. In some embodiments, the method reduces blood pressure in an ESRD subject on concomitant dialysis therapy, for example, pre-dialysis, post-dialysis, and/or interdialytic systolic and diastolic blood pressure may be reduced. In some embodiments, the method reduces interdialytic weight gain in an ESRD subject on concomitant dialysis therapy. In some embodiments, the subject also has heart failure. In some embodiments, one or more symptoms of intradialytic hypotension are improved after administration of a disclosed polymer, a composition comprising a disclosed polymer, a formulation, comprising a disclosed polymer, and/or a dosage form comprising a disclosed polymer, as disclosed herein. For example and without limitation, incidences of vomiting, fainting and/or drops in blood pressure levels are reduced or eliminated. In some embodiments, the subject experiences one or more of: a reduced frequency of emergency dialysis sessions, a reduced frequency of inadequate dialysis sessions, a reduced frequency of dialysis sessions on low-potassium dialysis bath, and/or reduced frequency or reduced severity of EKG signs during dialysis sessions. In some embodiments, one or more symptom of intradialytic hypotension are reduced or eliminated after administration of a polymer, a composition comprising a disclosed polymer, a composition comprising a disclosed polymer, a formulation comprising a disclosed polymer, and/or a dosage form comprising a disclosed polymer. Symptoms of intradialytic hypotension are known to those skilled in the art and may include, for example, vomiting, fainting, an abrupt decrease in blood pressure, seizures, dizziness, severe abdominal cramping, severe leg or arm muscular cramping, intermittent blindness, infusion, medication, and dialysis session interruption or discontinuation. In some embodiments, ESRD subjects may experience an improvement in physical function as expressed by increase in performance in the 6 Minute Walk Test.

In some embodiments, the disclosed polymers, compositions comprising the disclosed polymers, formulations comprising the disclosed polymers, and/or dosage forms comprising the disclosed polymers, as disclosed herein, are useful for treating a subject having chronic kidney disease. In some embodiments, the methods comprise administering to the subject an effective amount of the polymer, composition, formulation, and/or dosage form. In some embodiments, the polymers, compositions, formulations, and/or dosage forms may be co-administered with a base, as described herein. In some embodiments, the methods further comprise identifying the subject as having a chronic kidney disease before administration of a disclosed polymer, composition comprising the disclosed polymer, formulation comprising the disclosed polymer, and/or dosage form comprising a disclosed polymer, as disclosed herein. In some related embodiments, the methods further comprise reducing one or more symptoms of a fluid overload state in the subject. In some embodiments, a comorbidity of chronic kidney disease is reduced, alleviated, and/or eliminated after administration of a polymer, a composition comprising a disclosed polymer, a formulation comprising a disclosed polymer, and/or a dosage form comprising a disclosed polymer. Comorbidities of chronic kidney disease are known to those skilled in the art and include, for example, fluid overload, edema, pulmonary edema, hypertension, hyperkalemia, excess total body sodium, heart failure, ascites, and/or uremia. In some embodiments, CKD patients may experience prevention of doubling of serum creatinine over the duration of a study (for example, 1 to 2 years), prevention of disease progression to dialysis, and/or prevention of death and CKD related hospitalizations and/or complications.

In some embodiments, the disclosed polymers, compositions comprising the disclosed polymers, formulations comprising the disclosed polymers, and/or dosage forms comprising the polymers as, disclosed herein, are useful for treating a subject having hypertension. In some embodiments, the methods comprise administering to the subject an effective amount of the polymer, composition, formulation, and/or dosage form. In some embodiments, the polymers, compositions, formulations, and/or dosage forms may be co-administered with a base, as described herein. In some embodiments, the methods further comprise identifying that the subject has hypertension before administering a disclosed polymer, composition comprising a disclosed polymer, formulation comprising a disclosed polymer, and/or dosage form comprising a disclosed polymer, as disclosed herein. As used herein, the term hypertension includes the various subtypes of hypertension known to those skilled in the art, for example and without limitation: primary hypertension, secondary hypertension, salt sensitive hypertension, refractory hypertension, and combinations thereof. In some embodiments, the method is effective in reducing the subject's blood pressure. In related embodiments, the method may further comprise determining a blood pressure level before, after, or both before and after administration of the polymer, composition comprising a disclosed polymer, and/or dosage form comprising a disclosed polymer as disclosed herein. For example, the method may further comprise determining the subject's diastolic blood pressure, systolic blood pressure, and/or mean arterial pressure (“MAP”) before, after, or both before and after administration of the disclosed polymer, composition comprising the disclosed polymer, formulation comprising the disclosed polymer, and/or dosage form comprising the disclosed polymer, as disclosed herein. In some embodiments, one or more symptom of a fluid overload state is reduced, improved, or alleviated by administering a disclosed polymer, composition comprising a disclosed polymer, formulation comprising a disclosed polymer, and/or dosage form comprising a disclosed polymer, as disclosed herein. In some related embodiments, the method may further comprise determining a fluid overload state symptom before, after, or both before and after administration of a disclosed polymer, composition comprising a disclosed polymer formulation comprising a disclosed polymer, and/or dosage form comprising a disclosed polymer as disclosed herein. For example, the method may further comprise observing an improvement in the subject's breathing while lying down, ascites, fatigue, shortness of breath, body weight, peripheral edema, and/or pulmonary edema. In some embodiments, the subject is on concomitant diuretic therapy. As used herein, the term diuretic therapy refers to administration of pharmaceutical compositions (e.g., diuretic agents), and non-chemical intervention, such as dialysis or restriction of fluid intake. Diuretic agents are known to those skilled in the art and include, for example, furosemide, bumetanide, torsemide, hydrochlorthiazide, amiloride and/or spironolactone. In some related, embodiments, the diuretic therapy may be reduced or discontinued following administration of the disclosed polymer, composition comprising the disclosed polymer, formulation comprising the disclosed polymer, and/or dosage form comprising the disclosed polymer, as disclosed herein.

In some embodiments, the disclosed polymers, compositions comprising the disclosed polymers, formulations comprising the disclosed polymers, and/or dosage forms comprising the disclosed polymers, as disclosed herein, are useful for treating hyperkalemia in a subject. In some embodiments, the method comprises administering to the subject an effective amount of the polymer, composition, formulation, and/or dosage form as disclosed herein. In some embodiments, the polymers, compositions, formulations, and/or dosage forms may be co-administered with a base, as described herein. In some embodiments, the method further comprises identifying the subject as having hyperkalemia, or as having a risk of developing hyperkalemia, before administering the disclosed polymer, composition comprising a disclosed polymer, formulation, and/or dosage form comprising a disclosed polymer, as disclosed herein. In some embodiments, the method may further comprise determining a potassium ion level in the subject before administering the disclosed polymer, composition comprising the disclosed polymer, formulation comprising the disclosed polymer, and/or dosage form comprising a disclosed polymer, as disclosed herein. In some related embodiments, the potassium ion level may be within a normal range, slightly elevated, or elevated before administering the disclosed polymer, composition comprising the disclosed polymer, formulation comprising the disclosed polymer, and/or dosage form comprising the disclosed polymer, as disclosed herein. In some embodiments, the subject has been prescribed or will be administered a drug known to increase potassium levels. In some embodiments, the subject has already ingested a drug known to increase potassium levels. In some embodiments, the method may further comprise determining a second, reduced potassium ion level in the subject after administration of the disclosed polymer, composition comprising the disclosed polymer, formulation comprising the disclosed polymer, and/or dosage form comprising the disclosed polymer, as disclosed herein. In some embodiments, an acid/base balance associated with the subject does not change, for example, as measured by, serum total bicarbonate, arterial blood pH, urine pH, and/or urine phosphorous, after administration of the disclosed polymer, composition comprising the disclosed polymer, formulation comprising the disclosed polymer, and/or dosage form comprising the disclosed polymer, as disclosed herein.

In some embodiments, the disclosed polymers, compositions comprising the disclosed polymers, formulations comprising the disclosed polymers, and/or dosage forms comprising the disclosed polymers, as disclosed herein, are useful for treating an abnormally high sodium level, e.g., hypernatremia, in a subject. In some embodiments, the method comprises administering to the subject an effective amount of the polymer, composition, formulation, and/or dosage form as disclosed herein. In some embodiments, the disclosed polymers, compositions, formulations, and/or dosage forms may be co-administered with a base, as described herein. In some embodiments, the method further comprises identifying the subject as having an abnormally high sodium level, or as having a risk of developing an abnormally high sodium level, before administering the disclosed polymer, composition comprising the disclosed polymer, formulation comprising the disclosed polymer, and/or dosage form comprising the disclosed polymer, as disclosed herein. In some embodiments, the method may further comprise determining a sodium ion level, e.g., a total body sodium ion level, in the subject before administering the disclosed polymer, composition comprising the disclosed polymer, formulation comprising the disclosed polymer, and/or dosage form comprising the disclosed polymer, as disclosed herein. In some related embodiments, the sodium ion level, e.g., serum sodium ion level, may be within a normal range, slightly elevated, or elevated before administering the disclosed polymer, composition comprising the disclosed polymer, formulation comprising the disclosed polymer, and/or dosage form comprising the disclosed polymer, as disclosed herein. In some embodiments, the method may further comprise determining a second, reduced sodium ion level, e.g., a total body sodium ion level, in the subject after administration of the disclosed polymer, composition comprising the disclosed polymer, formulation comprising the disclosed polymer, and/or dosage form comprising the disclosed polymer, as disclosed herein. In some embodiments, an acid/base balance associated with the subject does not change, for example, as measured by, serum total bicarbonate, arterial blood pH, urine pH, and/or urine phosphorous, after administration of the disclosed polymer, composition comprising the disclosed polymer, formulation comprising the disclosed polymer, and/or dosage form comprising the disclosed polymer, as disclosed herein. In some embodiments, the subject has taken or will take a drug known to increase sodium levels, for example and without limitation: estrogen containing compositions, mineralocorticoids, osmotic diuretics (e.g., glucose or urea), vaptans (e.g., tolvaptan, lixivaptan), lactulose, cathartics (e.g., phenolphthalein), phenyloin, lithium, Amphotericin B, demeclocycline, dopamine, ofloxacin, orlistat, ifosfamide, cyclophosphamide, hyperosmolar radiographic contrast agents (e.g., gastrographin, renographin), cidofovir, ethanol, foscarnet, indinavir, libenzapril, mesalazine, methoxyflurane, pimozide, rifampin, streptozotocin, tenofir, triamterene, and/or choichicine. In some embodiments, administration of the disclosed polymer, compositions comprising the disclosed polymer, formulations comprising the disclosed polymer, and/or dosage forms comprising the disclosed polymer may further comprise increasing a dose of one or more additional agents, for example, an agent known to cause an increase in sodium levels. In some embodiments, the method further comprises increasing a dose of one or more of: an aldosterone antagonist, an angiotensin II receptor blocker, and/or an angiotensin-converting enzyme inhibitor before, concomitantly, and/or after administering a disclosed polymer, a composition comprising a disclosed polymer, a formulation comprising a disclosed polymer, and/or a dosage form comprising a disclosed polymer. In some embodiments, administration of the disclosed polymer, composition comprising a disclosed polymer, formulation comprising a disclosed polymer, and/or dosage form comprising a disclosed polymer may further comprise decreasing a dose or discontinuing administration or co-administration of a diuretic.

In some embodiments, the disclosed polymers, compositions comprising the disclosed polymers, formulations comprising the disclosed polymers, and/or dosage forms comprising the disclosed polymers, as disclosed herein, are useful for treating a subject with a disease or disorder involving fluid overload (e.g., a fluid overload state such as heart failure, end stage renal disease, ascites, renal failure, nephritis, and nephrosis). In some embodiments, the method comprises administering to the subject an effective amount of the polymer, composition, formulation, and/or dosage form as disclosed herein. In some embodiments, the polymers, compositions, formulations, and/or dosage forms may be co-administered with a base, as described herein. In some embodiments, the subject may be on concomitant diuretic therapy. In some embodiments, the method may further comprise identifying a fluid overload state in the subject, or identifying a risk that the subject will develop a fluid overload state before administration of a disclosed polymer, composition comprising a disclosed polymer, formulation comprising a disclosed polymer, and/or dosage form comprising a disclosed polymer. Methods of identifying a fluid overload state or a risk of developing a fluid overload state are known to those skill in the art and may include, for example and without limitation: assessing difficulty breathing when lying down, ascites, fatigue, shortness of breath, increased body weight, peripheral edema, and/or pulmonary edema associated with the subject. In some embodiments, an acid/base balance associated with the subject, for example, as measured by, serum total bicarbonate, arterial pH, urine pH, and/or urine phosphorous, does not change within about one day of administration of the disclosed polymer, composition comprising the disclosed polymer, formulation comprising the disclosed polymer, and/or dosage form comprising the disclosed polymer, as disclosed herein.

In some embodiments, the disclosed polymers, compositions comprising the disclosed polymers, formulations comprising the disclosed polymers, and/or dosage forms comprising the polymers, as disclosed herein, are useful for treating a subject with a disease or disorder involving fluid maldistribution (e.g., a fluid maldistribution state such as pulmonary edema, angioneurotic edema, ascites, high altitude sickness, adult respiratory distress syndrome, uticarial edema, papille edema, facial edema, eyelid edema, cerebral edema, and scleral edema). In some embodiments, the method comprises administering to the subject an effective amount of the polymer, composition, formulation, and/or dosage form, as disclosed herein. In some embodiments, the polymers, compositions, formulations, and/or dosage forms may be co-administered with a base, as described herein. In some embodiments, the method may further comprise identifying a fluid maldistribution state or a risk of developing a fluid maldistribution state in the subject before administering to the subject a disclosed polymer, composition comprising a disclosed polymer, formulation comprising a disclosed polymer, and/or dosage form comprising a disclosed polymer.

In some embodiments, the disclosed polymers, compositions comprising the disclosed polymers, formulations comprising the disclosed polymers, and/or dosage forms comprising the disclosed polymers, as disclosed herein, are useful for treating edema in a subject. In some embodiments, the method comprises administering to the subject an effective amount of the polymer, composition, formulation, and/or dosage form, as disclosed herein. In some embodiments, the polymers, compositions, formulations, and/or dosage forms may be co-administered with a base, as described herein. In some embodiments, the method may further comprise identifying an edematous state or a risk of developing an edematous state in the subject before administering a disclosed polymer, composition comprising a disclosed polymer, formulation comprising a disclosed polymer, and/or dosage form comprising a disclosed, as disclosed herein. In some embodiments, the edematous state is nephritic edema, pulmonary edema, peripheral edema, lymphedema, and/or angioneurotic edema. In some embodiments, the subject is on concomitant diuretic therapy. In some related embodiments, the diuretic therapy may be reduced or discontinued after administration of the disclosed polymer, composition comprising the disclosed polymer, formulation comprising the disclosed polymer, and/or dosage form comprising the disclosed polymer, as disclosed herein. In some embodiments, the method may further comprise, before administering a disclosed polymer, composition comprising a disclosed polymer, formulation comprising a disclosed polymer, and/or dosage form comprising a disclosed polymer, as disclosed herein, determining one or more of: a baseline level of one or more ions (e.g., sodium, potassium, lithium and/or magnesium) in the subject, a baseline total body weight associated with the subject, a baseline total body water level associated with the subject, a baseline total extracellular water level associated with the subject (e.g., a measure of the degree of edema in a particular site as evidenced by depth of pitting or extent of x-ray changes, and/or a baseline total intracellular water level associated with the subject. In some embodiments, the method may further comprise, after administering a disclosed polymer, composition comprising a disclosed polymer, formulation comprising a disclosed polymer, and/or dosage form comprising a disclosed polymer, as disclosed herein, determining one or more of: a second level of one or more ions in the subject, a second total body weight associated with the subject, a second total body water level associated with the subject, a second total extracellular water level associated with the subject, and/or a second total intracellular water level associated with said subject. In some embodiments, the second level is lower than the corresponding baseline level. In some embodiments, an acid/base balance associated with said subject, for example, as measured by, serum total bicarbonate, arterial pH, urine pH, and/or urine phosphorous, does not significantly change within about one day of administration of the disclosed polymer, composition comprising a disclosed polymer, formulation comprising a disclosed polymer, and/or dosage form comprising a disclosed polymer. In some embodiments, a blood pressure level associated with the subject after administration of the disclosed polymer, composition comprising a disclosed polymer, formulation comprising a disclosed polymer, and/or dosage form comprising a disclosed polymer is substantially lower than a baseline blood pressure level associated with the subject determined before administration of the disclosed polymer, composition comprising a disclosed polymer, formulation comprising a disclosed polymer, and/or dosage form comprising a disclosed polymer. In some embodiments, one or more symptoms of edema are reduced and/or eliminated following administration of a disclosed polymer, composition comprising a disclosed polymer, formulation comprising a disclosed polymer, and/or dosage form comprising a disclosed polymer, as disclosed herein. Symptoms of edema are known to those skilled in the art; some non-limiting examples include: difficulty breathing when lying down, shortness of breath, peripheral edema, and leg edema.

In some embodiments, the disclosed polymers, compositions comprising the disclosed polymers, formulations comprising the disclosed polymers, and/or dosage forms comprising the disclosed polymers according to the present disclosure are useful for treating ascites in a subject. In some embodiments, the method comprises administering to the subject an effective amount of the polymer, composition, formulation, and/or a dosage form, as disclosed herein. In some embodiments, the disclosed polymers, compositions, formulations, and/or dosage forms may be co-administered with a base, as described herein. In some embodiments, the method may further comprise identifying an ascitic state or a risk of developing an ascitic state in the subject. In some embodiments, the subject is on concomitant diuretic therapy. In some related embodiments, the diuretic therapy may be reduced or discontinued after administration of the disclosed composition. In some embodiments, the subject may have taken, or will take, a drug known to increase potassium levels.

In some embodiments, the disclosed polymers, compositions comprising the disclosed polymers, formulations comprising the disclosed polymers, and/or dosage forms comprising the disclosed polymers, as disclosed herein, are useful for treating nephrotic syndrome in a subject. In some embodiments, the method comprises administering to said subject an effective amount of the polymer, composition, formulation, and/or dosage form, as disclosed herein. In some embodiments, the disclosed polymers, compositions, formulations, and/or dosage forms may be co-administered with a base, as described herein. In some embodiments, the method further comprises identifying the subject as having nephrotic syndrome, or as having a risk of developing nephrotic syndrome, before administering the disclosed polymer, composition comprising the disclosed polymer, formulation comprising the disclosed polymer, and/or dosage form comprising the disclosed polymer. In some embodiments, the method may further comprise determining one or more of: a level of one or more ions (e.g., sodium, potassium calcium, lithium, and/or magnesium) in the subject, a total body weight associated with the subject, a total body water level associated with the subject, a total extracellular water level associated with the subject, and/or a total intracellular water level associated with the subject before administering the disclosed polymer, composition comprising the disclosed polymer, formulation comprising the disclosed polymer, and/or dosage form comprising the disclosed polymer. In some embodiments, the method may further comprise determining a second, lower level of one or more of: a level of one or more ions in the subject, a total body weight associated with the subject, a total body water level associated with the subject, a total extracellular water level associated with the subject, and/or a total intracellular water level associated with the subject after administering the disclosed polymer, composition comprising the disclosed polymer, formulation comprising the disclosed polymer, and/or dosage form comprising the disclosed polymer. In some embodiments, an acid/base balance associated with the subject, for example, as measured by, serum total bicarbonate, arterial pH, urine pH, and/or urine phosphorous, does not significantly change within about one day of administration of the disclosed polymer, composition comprising the disclosed polymer, formulation comprising the disclosed polymer, and/or dosage form comprising a disclosed polymer. In some embodiments, a blood pressure level associated with the subject after administration of the disclosed polymer, composition comprising the disclosed polymer, formulation comprising the disclosed polymer, and/or dosage form comprising the disclosed polymer is substantially lower than a baseline blood pressure level associated with the subject before the administration(s). In some embodiments, one or more symptoms of fluid overload is alleviated, reduced, or eliminated after administration of the disclosed polymer, composition comprising the disclosed polymer, formulation comprising the disclosed polymer, and/or dosage form comprising the disclosed polymer. In some related embodiments, the symptom may be one or more of: difficulty breathing when lying down, shortness of breath, peripheral edema, and/or leg edema. In some embodiments, the subject may be on concomitant diuretic therapy. In some related embodiments, the diuretic therapy may be reduced or eliminated after administration of the disclosed polymer, composition comprising the disclosed polymer, formulation comprising the disclosed polymer, and/or dosage form comprising the disclosed polymer.

In some embodiments, methods according to the present disclosure may further comprise administering to the subject an additional agent such as mannitol, sorbitol, calcium acetate, sevelamer carbonate (Renvela®), lanthanum carbonate, and/or sevelamer hydrochloride.

In some embodiments, methods according to the present disclosure may further comprise administering to the subject an agent known to increase potassium levels. As used herein, the term “an agent known to increase potassium levels” refers to agents that are known to cause an increase, are suspected of causing an increase, or are correlated with an increase in potassium levels, e.g., serum potassium levels, upon administration. For example and without limitation, agents known to cause an increase in potassium levels may include: a tertiary amine, spironolactone, fluoxetine, pyridinium and its derivatives, metoprolol, quinine, loperamide, chlorpheniramine, chlorpromazine, ephedrine, amitryptyline, imipramine, loxapine, cinnarizine, amiodarone, nortriptyline, a mineralocorticosteroid, propofol, digitalis, fluoride, succinylcholine, eplerenone, an alpha-adrenergic agonist, a RAAS inhibitor, an ACE inhibitor, an angiotensin II receptor blocker, a beta blocker, an aldosterone antagonist, benazepril, captopril, enalapril, fosinopril, lisinopril, moexipril, perindopril, quinapril, ramipril, trandolapril, candesartan, eprosartan, irbesartan, losartan, valsartan, telmisartan, acebutolol, atenolol, betaxolol, bisoprolol, carteolol, nadolol, propranolol, sotalol, timolol, canrenone, aliskiren, aldosterone synthesis inhibitors, and/or VAP antagonists. In some embodiments, administration of the disclosed polymers, compositions comprising the disclosed polymers, formulations comprising the disclosed polymers, and/or dosage forms comprising the disclosed polymers may further comprise increasing a dose of one or more additional agents, for example, an agent known to cause an increase in potassium levels. In some embodiments, administration of the polymers, compositions comprising the disclosed polymers, and/or dosage forms comprising the disclosed polymers may further comprise decreasing a dose or discontinuing administration or co-administration of a diuretic, for example, as a result of having treated fluid overload with a disclosed polymers, compositions comprising the disclosed polymers, formulations comprising the disclosed polymers, and/or dosage forms comprising the disclosed polymers, as disclosed herein.

In some embodiments, methods according to the present disclosure may further comprise administering to the subject an agent known to increase sodium levels. As used herein, the term “an agent known to increase sodium levels” refers to agents that are known to cause an increase, are suspected of causing an increase, or are correlated with an increase in sodium levels upon administration. For example and without limitation, agents known to cause an increase in sodium levels may include: estrogen containing compositions, mineralocorticoids, osmotic diuretics (e.g., glucose or urea), vaptans (e.g., tolvaptan, lixivaptan), lactulose, cathartics (e.g., phenolphthalein), phenyloin, lithium, Amphotericin B, demeclocycline, dopamine, ofloxacin, orlistat, ifosfamide, cyclophosphamide, hyperosmolar radiographic contrast agents (e.g., gastrographin, renographin), cidofovir, ethanol, foscarnet, indinavir, libenzapril, mesalazine, methoxyflurane, pimozide, rifampin, streptozotocin, tenofir, triamterene, and/or cholchicine. In some embodiments, administration of the polymers, compositions comprising the disclosed polymers, and/or dosage forms comprising the disclosed polymers may further comprise increasing a dose of one or more additional agents, for example, an agent known to cause an increase in sodium levels. In some embodiments, administration of the polymers, compositions comprising the disclosed polymers, and/or dosage forms comprising the disclosed polymers may further comprise decreasing a dose or discontinuing administration or co-administration of a diuretic.

In some embodiments, methods according to the present disclosure may further comprise determining a baseline level of one or more ions in a subject before administering a disclosed polymer, composition comprising a disclosed polymer, formulation comprising a disclosed polymer, and/or the dosage form comprising a disclosed polymer, as disclosed herein, and determining a second level of the one or more ions in the subject after administering the disclosed polymer, composition comprising the disclosed polymer, formulation comprising the disclosed polymer, and/or dosage form comprising the disclosed polymer, as disclosed herein. In related embodiments, a baseline level of potassium is determined in a subject. In another embodiment, a baseline level of sodium is determined in a subject. Thereafter, a disclosed polymer, composition comprising a disclosed polymer, formulation comprising a disclosed polymer, and/or dosage form comprising a disclosed polymer, as disclosed herein, is administered to the subject, followed by a determination of a second potassium and/or sodium level. In some embodiments, the second potassium and/or sodium level is lower than the baseline potassium level.

In some embodiments, methods according to the present disclosure may further comprise determining a baseline total body weight associated with a subject before administering a disclosed polymer, composition comprising a disclosed polymer, formulation comprising a disclosed polymer, and/or dosage form comprising a disclosed polymer, as disclosed herein, and determining a second total body weight associated with the subject after administering the disclosed polymer, composition comprising the disclosed polymer, formulation comprising the disclosed polymer, and/or dosage form comprising the disclosed polymer, as disclosed herein. In some embodiments, the second total body weight is lower than the baseline total body weight. Any suitable method for determining the total body weight associated with a subject may be used.

In some embodiments, methods according to the present disclosure may further comprise determining a baseline total water level, e.g., total body water level, associated with a subject before administering a disclosed polymer, composition comprising a disclosed polymer, formulation comprising a disclosed polymer, and/or dosage form comprising a disclosed polymer, as disclosed herein, and determining a second total water level, e.g., total body water level, associated with the subject after administering the disclosed polymer, composition comprising the disclosed polymer, and/or dosage form comprising the disclosed polymer, as disclosed herein. In some embodiments, the second total water level, e.g., total body water level, is lower than the baseline total water level, e.g., total body water level. Any suitable method for determining a total water level associated with a subject may be used, for example, by bioimpedance measurement, or through invasive procedures, such as central vein catheters for measurement of pulmonary wedge pressure.

In some embodiments, methods according to the present disclosure may further comprise determining a baseline total extracellular water level associated with a subject before administering a disclosed polymer, composition comprising a disclosed polymer, formulation comprising a disclosed polymer, and/or dosage form comprising a disclosed polymer, as disclosed herein, and determining a second total extracellular water level associated with the subject after administering the polymer, composition comprising the disclosed polymer, formulation comprising the disclosed polymer, and/or dosage form comprising the disclosed polymer, as disclosed herein. In some embodiments, the second total extracellular water level is lower than the baseline total extracellular water level. Any suitable method for determining a total extracellular water level associated with a subject may be used, for example, by bioimpedance measurement, or through invasive procedures, such as central vein catheters for measurement of pulmonary wedge pressure.

In some embodiments, methods according to the present disclosure may further comprise determining a baseline total intracellular water level associated with a subject before administering a disclosed polymer, composition comprising a disclosed polymer, formulation comprising a disclosed polymer, and/or dosage form comprising a disclosed polymer, as disclosed herein, and determining a second total intracellular water level associated with the subject after administering the disclosed polymer, composition comprising the disclosed polymer, formulation comprising the disclosed polymer, and/or dosage form comprising the disclosed polymer, as disclosed herein. In some embodiments, the second total intracellular water level is lower than the baseline total intracellular water level. Any suitable method for determining a total intracellular water level associated with a subject may be used, for example, by bioimpedance measurement, or through invasive procedures, such as central vein catheters for measurement of pulmonary wedge pressure.

In some embodiments, methods according to the present disclosure may further comprise determining a pH level associated with a subject. Any method known in the art for determining a pH level may be employed. For example and without limitation, a pH level associated with a subject may be determined by determining the subject's pCO₂, serum carbonate, serum pH level, urinary phosphorous level, etc. In some embodiments, methods according to the present disclosure comprise determining a pH level associated with a subject after administering a disclosed polymer, composition comprising a disclosed polymer, formulation comprising a disclosed polymer, and/or dosage form comprising a disclosed polymer, according to the present disclosure. In related embodiments, the pH level is within a normal range for the subject, and/or within a clinically acceptable range for the subject. In some embodiments, a pH level associated with a subject after administering a polymer, composition comprising a polymer, and/or dosage form comprising a polymer according to the present disclosure is closer to a normal level for the subject, closer to a clinically acceptable level, etc., than compared to a baseline pH level associated with the subject before administration of the composition. In some embodiments, a pH level associated with the subject does not significantly change within about 1 day, within about 18 hours, within about 12 hours, within about 6 hours, within about 4 hours, or within about 2 hours of administration of the composition.

In some embodiments, methods according to the present disclosure may further comprise determining an acid/base balance associated with a subject. Any method known in the art for determining an acid/base balance may be employed. In some embodiments, methods according to the present disclosure comprise determining an acid/base balance associated with a subject after administering a composition according to the present disclosure. In related embodiments, the an acid/base balance is within a normal range for the subject, and/or within a clinically acceptable range for the subject. In some embodiments, an acid/base balance associated with a subject after administering a disclosed polymer, composition comprising a disclosed polymer, formulation comprising a disclosed polymer, and/or dosage form comprising a disclosed polymer, according to the present disclosure, is closer to a normal level for the subject, closer to a clinically acceptable level, etc., than compared to a baseline an acid/base balance associated with the subject before administration of the polymer, composition, formulation, and/or dosage form. In some embodiments, an acid/base balance associated with the subject does not change or significantly change within about 1 day, within about 18 hours, within about 12 hours, 10 hours, within about 9 hours, within about 8 hours, within about 7 hours, within about 6 hours, within about 5 hours, within about 4 hours, within about 3 hours, within about 2 hours, or within about 1 hour of administration of the composition.

Methods for determining an ion level in a subject are known to those skilled in the art. Any suitable method for determining an ion level may be used. However, determination of serum sodium levels should be avoided as such levels tend not to fluctuate, even in hypernatremic subjects. If sodium ion levels are desired, another suitable method for determining such levels should preferably be used, such as determining a subject's total body sodium level.

In some embodiments, methods according to the present disclosure may further comprise determining a blood pressure level before, after, or both before and after administration of a disclosed polymer, composition comprising a disclosed polymer, formulation comprising a disclosed polymer, and/or dosage form comprising a disclosed polymer, according to the present disclosure. A subject's blood pressure level may be determined using any suitable method known in the art. For example and without limitation, a subject's blood pressure level may be determined by measuring the subject's systolic blood pressure, the subject's diastolic blood pressure, and/or the subject's mean arterial pressure (“MAP”). In some embodiments, the subject's blood pressure is lower after treatment than before treatment.

In some embodiments, the disclosed polymers, compositions comprising the disclosed polymers, formulations comprising the disclosed polymers, and/or dosage forms comprising the disclosed polymers, according to the present disclosure, are administered as needed to reduce an ion level in a subject, and/or to maintain an acceptable level of one or more ions in a subject, and/or to reduce a fluid overload state or fluid maldistribution state in a subject. In some embodiments, compositions according to the present disclosure are administered at a frequency from 1 time per every 3 days to about 4 times per day. Preferably, the compositions according to the present disclosure are administered from about 1 time per day to about 4 times per day; for example, once or twice per day.

### EXAMPLES

The following examples are for illustrative purposes only and are not to be construed as limiting in any manner.

### Example 1

This example demonstrates the preparation of an exemplary cross-linked polyelectrolyte polymer, such as crosslinked polyacrylic acid partially neutralized with sodium.

An inverse suspension process may be used with the following components: a monomer (e.g., acrylic acid), solvent for the monomer (e.g., hydrophilic, for example, water), base for neutralization of monomer (e.g., NaOH), lipophilic (e.g., hydrophobic) solvent (e.g., Isopar™ L), suspending agent (e.g., fumed silica such as Aerosil R972), chelating agent (e.g., Versenex™-80), polymerization initiator (e.g., sodium persulfate), and cross-linking agent (e.g., TMPTA).

A monomer solution is prepared in a vessel as the aqueous phase by dissolving an unsaturated carboxylic acid monomer (e.g., acrylic acid) in water and neutralizing with an aqueous alkali (e.g., NaOH) to a desired percentage neutralization (e.g., 70% to 95% neutralized). Just before addition of this aqueous, partially neutralized, monomer solution to the reactor, one or more polymerization initiators (e.g., sodium persulfate alone or a redox-couple, such as t-butylhydroperoxide paired with thiosulfate) are added under conditions that do not favor polymerization. Optionally, a chelating agent (e.g., Versenex™-80) can be added to the aqueous mixture ensure control of transition metal ions. An organic phase (e.g., Isopar™ L or toluene or n-heptane or cyclohexane) is placed into the main reactor (not the vessel with the aqueous monomer solution). A hydrophobic suspending agent (e.g., Aerosil R972) is dissolved or dispersed in the organic phase. A crosslinking agent is added. If the crosslinking agent is soluble in the organic phase (e.g., divinylbenzene or 1,1,1-trimethylolpropane triacrylate—also called TMPTA), it is added to the reactor with the organic phase. If the crosslinking agent is water soluble (e.g., highly-ethoxylated trimethylolpropane triacrylate—also called HE-TMPTA—or diacryl glycerol), the crosslinking agent is added to the aqueous phase. The aqueous phase is then added to the organic phase in the reactor, e.g., with mixing, and the reaction mixture is agitated to produce aqueous droplets of the appropriate size in the organic solvent. Simultaneously, oxygen is removed from the reaction mixture by bubbling an inert gas (e.g., nitrogen) through the reaction mixture. After adequate deoxygenation, the reaction will either begin (e.g., in the case of redox couples) or be started by increasing the temperature (e.g., in the case of sodium persulfate). A second addition of hydrophobic suspending agent may be added as the polymerization proceeds, i.e., to further stabilize the particles. Reaction is completed by maintaining an elevated temperature (e.g., 65° C.) for a time adequate to allow removal, i.e., reaction of substantially all of the monomer (e.g., 2 to 4 hours). Water may then be removed by azeotropic distillation and the crosslinked cation-binding polymeric material may be isolated by filtration or centrifugation to remove the remaining organic solvent. The polymeric material may be rinsed with fresh organic solvent and dried to the desired moisture and/or organic solvent content as measured by loss on further drying. In some embodiments, less than 500 ppm of the monomer remains after polymerization. The polymer may be rinsed to remove this residual monomer.

In an exemplary method, acrylic acid (140 g) was added dropwise to a solution of 124.35 g of 50% NaOH and 140 g of deionized water while keeping the temperature below 40° C. to prevent initiation of polymerization. 3.5 g of Versenex™ 80 and 0.70 g of a 10% solution of sodium persulfate were added. Meanwhile, 1200 g of Isopar™ L were charged into the main reactor. 0.80 g Aerosil R972 dissolved in 40 g of Isopar™ L and 0.50 g of TMPTA were added to the main reactor. The aqueous monomer solution was added to the reactor, which was then closed. Agitation was started at 330 RPM and argon was bubbled through the reaction mixture. After 70 minutes of bubbling argon, the reaction was heated rapidly at 4° C. increase per minute. When the temperature reached 50° C., another 0.80 g of Aerosil R972 in 40 g of Isopar™ L (that had been separately bubbled with argon) was added to the reaction mixture. The reaction exotherm heated the mixture to 80° C. over the next 15 minutes while the constant temperature bath was removing heat to keep the reaction mixture at 65° C. The reaction mixture cooled to 70° C. at approximately 60 minutes from the start of heating. The reaction mixture was kept at 65° C. to 70° C. for 4 hours. The reaction mixture was allowed to cool. The resulting crosslinked cation-binding polymer was isolated by filtration and dried in vacuum at 105° C.

### Example 2

This example illustrates the preparation of an exemplary crosslinked polyelectrolyte polymer by an aqueous phase reaction of a partially neutralized carboxylic acid monomer.

A monomer solution is prepared in a reactor by dissolving an unsaturated carboxylic acid monomer (e.g., acrylic acid) in water and neutralizing with an aqueous alkali (e.g., NaOH) to a desired percentage neutralization (e.g., 70 to 95 percent neutralized). Optionally, a chelating agent (e.g., Versenex™ 80) may be added to control metal ions. A suitable crosslinking agent (e.g., 1,1,1-trimethylolpropane triacrylate or diacryl glycerol) is added to the reactor. A polymerization initiator is added to the reactor. The reactor is then closed and the reaction mixture is bubbled with an inert gas (e.g., nitrogen) and agitated until adequate removal of oxygen is achieved. The reaction is then initiated either by reaching an oxygen concentration where a redox couple produces radicals or by adding heat to cause a temperature dependent initiator (e.g., persulfate salts) to produce radicals. The reaction is allowed to proceed through the exothermic heating that occurs during reaction. After 2 to 6 hours, the reaction is completed and the gel-like mass of reaction product can be removed from the reactor and cut into appropriately sized pieces. After drying, the particles can be separated by size or milled to produce the desired size or size distribution.

Thus, in an exemplary method, 140 g of acrylic acid were added dropwise to a solution of 124.35 g of 50% NaOH and 140 g of deionized water while keeping the temperature below 40° C. to prevent initiation of polymerization. Then, 3.5 g of Versenex™ 80 and 0.70 g of a 10% solution of sodium persulfate were added. The final addition was 0.50 g of TMPTA. The reactor was closed and the reaction mixture agitated at 200 RPM while argon was bubbled through the mixture. After 70 minutes of bubbling argon, the reaction was initiated by heating at a rate of a 4° C. temperature rise per minute. After 7 minutes, the reaction reached 55° C. and the entire reaction mixture became a gel. The agitation was stopped, allowing the gel to slowly settle to the bottom of the reactor. The temperature of the heating bath was maintained at 65° C. for another 4 hours. The gel was then cooled, cut into pieces, and dried in a vacuum at 105° C.

### Example 3

This example illustrates the conversion of a partially sodium-substituted crosslinked polycarboxylic polymer prepared, for example, according to Example 1 or 2, to a crosslinked polycarboxylic acid polymer with a reduced degree of sodium substitution (e.g., an acidified polymer).

The polymer is weighed and the relative content of different cations (either from knowledge of the preparation or, more preferably, from elemental analysis of a sample) is used to determine the number of moles of carboxylate present. The polymer is then washed with an excess (e.g., twice the number of moles of carboxylates, or more) of 1 N acid (preferably HCl), either in batches or by column elution. The resulting acidified polymer is rinsed with water to remove any excess of the 1 N acid, and dried in a vacuum at 60° C.

For example, 89.65 g of a polymer produced by the technique of Example 1 were placed into a beaker and stirred with 667 mL of 1 N HCl for 2 hours. The liquid was drained and the polymeric particles were returned to the vessel. A second aliquot of 667 mL of 1 N HCl was added and the mixture was stirred for 1 hour. The liquid was drained and a third rinse with 667 mL of 1 N HCl was performed for 1 hour. The liquid was drained and the polymeric material was placed into 667 mL of deionized water and stirred for 1 hour. The liquid was drained and another 667 mL of deionized water was added. The polymeric material was then stirred for 1 hour before draining the liquid. This water washing was continued until the pH of the rinse water was above 3. The crosslinked cation-binding polymer was then dried in a vacuum at 60° C.

Alternatively, one-hundred grams of a cross-linked polyelectrolyte polymer, such as a partially neutralized cross-linked polyacrylate polymer (e.g., prepared as described in Example 1 above) was placed into a vessel. Next, about 2,250 milliliters of pure (e.g., trace metal or otherwise certified low metal) 1 M HCl was added to the vessel and then the polymer and the acid were stirred gently for two hours. The liquid was removed by decanting or filtration. If desired due to vessel size or for improved mass balance, the 2,250 milliliters of 1M HCl is divided into multiple batches and used sequentially. For instance, 750 milliliters were added, stirred with the polymer, and removed followed by two or more separate additions of 750 milliliters. The polymer was then rinsed with 2,250 milliliters of low metal content water to remove excess acid surrounding the polyelectrolyte such as a polyacrylate. The crosslinked cation-binding polymer was then dried.

Further alternatively, one-hundred grams of a cross-linked polyelectrolyte polymer, such as a cross-linked polyacrylate polymer were placed into a filtration funnel or a column equipped with a bottom filter. The polymer was then rinsed with about 2,250 milliliters of pure (e.g., trace metal or otherwise certified low metal) 1 M HCl for about an hour or more. Next, the polymer was rinsed with 2,250 milliliters of low metal content water. The crosslinked cation-binding polymer was then dried.

Exemplary acidified polymers useful as crosslinked cation-binding polymers prepared according to this Example generally have a saline holding capacity of greater than about 40 g/g (see, e.g., Examples 8 and 9); and contain less than about 5,000 ppm of sodium, less than about 20 ppm of heavy metals, less than about 500 ppm of residual monomer, less than about 2,000 ppm of residual chloride, and less than about 20 wt. % of soluble polymer. Preferably, acidified polymers useful as crosslinked cation-binding polymers prepared according to this Example have a saline holding capacity of greater than about 80 g/g (see, e.g., Examples 5 and 6); and contain less than about 500 ppm of sodium, less than about 20 ppm of heavy metals, less than about 50 ppm of residual monomer, less than about 1,500 ppm of residual chloride, and less than about 10 wt. % of soluble polymer. Crosslinked cation-binding polymers prepared according to the method of Example 1 (using acrylic acid monomers) and acidified to prepare the exemplary acidified polymers of the present Example may be referred to as “H—CLP” or “HCLP”.

### Example 4

This example demonstrates the preparation of substantially metal free (e.g., acid form) cross-linked polyelectrolyte polymers, such as cross-linked polyacrylic acid polymer.

In an exemplary method, substantially metal free (e.g., acid form) cross-linked polyacrylic acid polymer was prepared by placing 140 g of glacial acrylic acid (e.g., not neutralized as in Example 1) into a three to five liter reactor with 2,200 to 2,500 milliliters of dilute acid, such as 1 M HCl. A water soluble cross linking agent, such as 1,3-diglycerate diacrylate, in a ratio chosen to produce the desired saline holding capacity (e.g., 20-fold, 30-fold, 40-fold or more) and an initiator were added to the monomer solution. After sparging the reactor with an inert gas, (e.g., nitrogen) and agitating the reaction mixture, the reaction was started and allowed to proceed for two to four hours until substantially all of the monomer had reacted. The resultant mass of wet polymer was then cut into smaller pieces (e.g., 1-2 cm per side), dried in a vacuum or in an inert atmosphere, and then disrupted (e.g., by milling) to produce particles or powder.

140 g of acrylic acid was placed into a reactor and diluted with 326 g of deionized water followed by addition of 0.50 g of TMPTA and 0.70 g of a 10% solution of sodium persulfate. The reactor was closed and the reaction mixture was agitated at 250 RPM while argon was bubbled through the reaction mixture. After 70 minutes of bubbling argon, the reaction mixture was heated to produce approximately 4° C. increase in temperature per minute. After 7 minutes, the temperature reached approximately 50° C. and the entire reaction mixture became a gel that quickly settled to the bottom of the reactor when the agitation was stopped. Heating at 65° C. was continued for 2 hours and the gel was allowed to cool overnight. The gel was then cut into pieces and dried in a vacuum oven at 60° C. The resultant mass of polymer was then cut into smaller pieces (e.g., 1-2 cm per side), dried in a vacuum or in an inert atmosphere, and then disrupted (e.g., by milling) to produce particles or powder.

Free-acid forms of crosslinked cation-binding polymers prepared according to the present example represent alternative forms of H—CLP.

### Example 5

This example describes the preparation of Ca—CLP from H—CLP.

In an exemplary method, Ca—CLP was prepared by adding 60 L of deionized water to a 100 L jacketed vessel and then heating to 65° C. An appropriate amount of CaO (for example, 390 g of CaO for preparation of polymer containing 25% calcium counterions) was added slowly with stirring and the mixture stirred to create a solution or suspension. 4 kg of HCLP (for example HCLP prepared by a process substantially similar to Example 3 from NaCLP prepared by process substantially similar to Example 1) was then added to the solution and the mixture stirred at 65° C. for four hours. After these four hours, any free water was poured off. The resulting calcium containing polymer (termed “CaCLP” or “Ca—CLP” herein) was then transferred to drying trays and dried in a tray drier at 100° C. until the water content was less than 5%. For example, for preparation of 25% CaCLP, 390 g of CaO was used.

Alternatively, other calcium bases (for example, CaCO₃) or calcium salts (for example, CaCl₂) may be used as the calcium source for preparation of CaCLP from HCLP. For example, polymer partially neutralized with sodium base, as described in Examples 1 and 2, may be hydrated with water and equilibrated with a calcium salt (e.g. CaCl₂) solution to exchange the sodium with calcium. This equilibration may be repeated with fresh solutions of calcium salt to effect more complete exchange with sodium and to remove sodium prior to drying.

### Example 6

The content (e.g., percentage; %) of certain cations (e.g., calcium, sodium, magnesium, and/or potassium) on a polymer may be determined by ICP-AES and ICP-MS, for example, with a ThermoElectron Finnegan Element 2 or a Perkin Elmer Elan 6000 instrument. The percentage of cations that are counterions to the carboxylate groups in the polymer determined in different ICP measurements may vary by ±20% or less. For example, the determination of 15% to 35% calcium cations as counterions to carboxylate groups in the polymer may vary in different measurements by ICP (e.g., 15%±20% to 35%±20%).

For example, the calcium and/or sodium content of a polymer prepared according to Example 5 can be determined by diluting a 250 mg sample of the polymer with 5% nitric acid solution to a total volume of 100 mL. After shaking overnight to extract the calcium and sodium cations from the polymer, an aliquot of the mixture can be diluted with a 1% nitric acid solution as necessary to bring the concentration of the cation within the range of a suitable calibration curve. To ensure complete digestion of the sample, an exemplary method is to fully digest the sample in nitric acid (e.g., until the solution becomes clear and colorless), for example by application of heat; using microwave digestion; using other acids or mixture of acids, hydrogen peroxide, or other reagents; or by other methods known in the art. For example, when using ICP-AES, such as with a ThermoElectron Finnegan Element 2 instrument, a 10-fold dilution is used for sodium determinations and a 100-fold dilution is used for calcium determinations. For example, when using ICP-MS, such as with a Perkin Elmer Elan 6000 or a ThemoElement2 instrument, a 10-fold dilution is used for sodium determinations and a 10,000-fold dilution is used for calcium determinations. The final dilution volume should be 10.0 mL. In order to normalize the results of multiple runs, an internal standard such as scandium or germanium (e.g., about 100 μL of a 10,000 μg/mL solution of 99.999% scandium oxide in 5% nitric acid) was added to the 10-mL diluted samples before analysis.

In an exemplary method, a 250.08 mg sample of a polymer prepared according to Example 5 was placed in a 100-mL polypropylene tube and a 5% nitric acid solution was added until the total volume of the sample was 100 mL. The tube was then shaken overnight to produce “Mixed Sample A.” A 250.11 mg sample of the same polymer used to prepare Mixed Sample A was placed in a 100-mL polypropylene tube and a 5% nitric acid solution was added until the total volume of the sample was 100 mL. The tube is then shaken overnight to produce “Mixed Sample B.” Next, three 0.100-mL aliquots Mixed Sample A were diluted with a 1% nitric acid solution to final volumes of 10.0 mL. As an internal standard, 102 μL, 101 μL, and 100 μL of a 10,000 μg/mL standard solution of 99.999% scandium oxide in 5% nitric acid was added to the three aliquots, respectively. Separately, three 0.100-mL aliquots of Mixed Sample B were similarly diluted with 1% nitric acid to final volumes of 10.0 mL and doped with 100 μL, 99.0 and 100 μL of the standard scandium solution, respectively. Analysis of calcium content proceeded using a ThermoElectron Finnigan Element 2 ICP-AES instrument (equipped with software version 2.42) according to the manufacturer's specifications. The six raw calcium concentration measurements (e.g., 55,449, 55,318, 54,761, 56,079, 56,375, and 55,949 μg/g, respectively) were determined by normalizing the intensity of the raw calcium measurement to the measurement of the internal scandium standard. These six raw calcium concentration measurements were then converted into weight percent values (e.g., 5.54, 5.53, 5.48, 5.61, 5.64, and 5.59 wt. % Ca, respectively) and averaged to provide an overall calcium content of 5.6 wt. %. The percentage of carboxylate groups to which calcium serves as a counterion on a polyacrylate polymer (e.g., the “[x]% Ca—CLP” nomenclature) can be determined from the weight percent calcium measurement (wt. % Ca) by the following equation:

[x]% Ca—CLP=(72.06)(wt. % Ca)/(20.05−(0.19)(wt. % Ca))

For this example analysis, therefore, the polymer would be termed “21% Ca—CLP.” Using Mixed Sample A and Mixed Sample B described in the previous paragraph, sodium content was determined by ICP-AES as follows. Three 1.0-mL aliquots of Mixed Sample A were each diluted to a final volume of 10.0 mL using 1% nitric acid solution. To these were each added 113 μL of a 10,000 μg/mL standard solution of 99.999% scandium oxide in 5% nitric acid. Similarly, three 1.00-mL aliquots of Mixed Sample B were diluted to final volumes of 10.0 mL and were doped with 115 μL, 115 μL, and 116 μL of the standard scandium solution. Analysis of sodium content proceeded using a ThermoElectron Finnigan Element 2 ICP-AES instrument (equipped with software version 2.42) according to the manufacturer's specifications. The six raw sodium concentration measurements (e.g., 327, 328, 328, 381, 381, and 381 μg/g, respectively) were determined by normalizing the intensity of the raw sodium measurement to the measurement of the internal scandium standard. These six raw sodium concentration measurements were then averaged (354 μg/g) wherein:
354 μg/g is equivalent to 0.035 wt %

The percentage of carboxylate groups to which sodium serves as a counterion (e.g., the “[x]% Na—CLP” nomenclature) on a polyacrylate polymer can be determined from the weight percent sodium measurement (wt. % Na) by the following equation:

[x]% Na—CLP=(72.06)(wt. % Na)/(23.0−(0.23)(wt. % Na))

For this example analysis, with an average sodium concentration of 354 ug of sodium per gram of polymer, or 0.035 wt. % sodium, sodium cations are counterions to about 0.11% of the carboxylate groups in the polymer.

The percentage of carboxylate groups to which magnesium serves as a counterion on a polyacrylate polymer (e.g., the “[x]% Mg—CLP” nomenclature) can be determined from the weight percent measurement (wt. % Mg) by the following equation:

[x]% Mg—CLP=(72.06)(wt. % Mg)/(12.15−(0.11(wt. % Mg))

In another exemplary method, the content of certain cations (e.g., calcium, sodium, magnesium, potassium or other cations) on a polymer may be determined by ICP-OES. For example, the calcium content of a polymer prepared according to Example 5 can be determined by diluting a measured mass of polymer with a known volume of a 5% aqueous solution of trace metal grade nitric acid. The sample is then digested by first heating the polymer mixture until gaseous NO₂ is apparent. While continuing to heat, a small measured aliquot of 30-40% hydrogen peroxide is added to the solution. The solution foams and may turn brown. Once the foaming subsides an additional aliquot of hydrogen peroxide is added and repeated until the foaming after hydrogen peroxide addition is minimal, no particulate is visible, and a clear and colorless solution has been prepared. The total volume of hydrogen peroxide is recorded. Additional measured volumes of 5% nitric acid may be added during the digestion process to maintain an adequate volume of liquid. An appropriate volume of the digested polymer sample is diluted to a final volume of 10 mL with the 5% nitric acid solution to bring the concentration of the cation within the range of a suitable calibration curve; serial dilutions in 5% nitric acid can be made with the total dilution recorded. An internal scandium/cesium standard/ionization buffer was prepared from CsNO₃ and a scandium standard and was used in all analyses to normalize results and correct for matrix effects. The internal standard was prepared by adding 50 mg scandium standard (1000 μg/mL) and 1.48 g anhydrous CsNO3 to 1 L of 5% trace metal grade nitric acid. The internal was mixed with the sample online prior to injection into the ICP instrument. Standard solutions for construction of the standard curve were prepared at 0.2, 1, 5 and 25 ug/g Ca in 5% nitric acid. Samples were analyzed by ICP-OES on a Perkin Elmer Optima 5300 DV. Ca concentrations in μg/g were determined from the standard curve with correction for dilution, and converted to weight percent as described above.

### Example 7

The effect of % calcium-CLP on the bioadhesion of hydrated polymer was assessed. For these studies, H—CLP is prepared, for example, as described in Examples 1 and 3.

Calcium oxide was added to each sample in the amount shown in Table 3 to achieve degrees of % calcium-CLP, e.g., 5 to 40%. After equilibration with stirring excess solution was drained from the Ca—CLP. The hydrated polymer was used for the bioadhesiveness determination.

Samples of each calcium loaded bead preparation were placed in glass beakers and dried in a vacuum oven. These samples were analyzed for calcium content using an Inductively Coupled Plasma optical emission spectroscopy (ICP-OES) method, for example, as described in Example 6.

TABLE 3
CaCLP Sample Preparation Summary

| | Weight of | |
|---|---|---|
| Target % Ca | Added CaO, | Actual % Ca |
| Substitution | g | Substitution |
| 0 | 0.0000 | 1 |
| 5 | 0.0782 | 5 |
| 10 | 0.1570 | 11 |
| 15 | 0.2408 | 18 |
| 20 | 0.3173 | 24 |
| 25 | 0.3963 | 28 |
| 30 | 0.4698 | 32 |
| 35 | 0.5492 | 37 |
| 40 | 0.6317 | 41 |

The bioadhesiveness of the polymer samples was assessed using skin and the results are summarized in Table 4. In the hydrated state, 0% Ca—CLP (H—CLP) was bioadhesive to skin. In contrast, 10% to 40% Ca—CLP was not bioadhesive to skin.

TABLE 4
Stickiness of CaCLP in Hydrated Stage
vs. Degree of Ca Substitution

| | Bioadhesiveness in |
|---|---|
| Target % | Hydrated State |
| Ca-CLP | Skin (finger) |
| 0 | Yes |
| 5 | Yes |
| 10 | No |
| 15 | No |
| 20 | No |
| 25 | No |
| 30 | No |
| 35 | No |
| 40 | No |

### Example 8

The saline holding capacity of a cross-linked polyelectrolyte polymer, such as a cross-linked polyacrylate polymer, may be determined by known methods in the art.

In an exemplary method, saline holding capacity for H—CLP was determined with a 0.15 M sodium phosphate buffered solution as follows. A pH seven buffer of sodium phosphate tribasic (Na₃PO₄.12H₂O; MW 380.124) was prepared by dissolving 19.0062 grams in about 950 milliliters pure water and adjusting the pH to a final pH of 7±0.1 with 1N HCl before final dilution to one liter resulting in a solution with a sodium concentration of 0.15 M. Next, an amount of cross-linked cation-binding polyelectrolyte, for example, cross-linked polyacrylate CLP particles (e.g., HCLP prepared according to Examples 1-4) (e.g., 0.1±0.025 grams), were transferred to a tared filter tube and the mass of the polymer was recorded as in W1. Next, the tube was returned to the balance to record the weight of the tube plus the sample as W2. An excess (e.g., more than seventy times the mass of polymer) amount of the pH 7.0 buffer (e.g., ten milliliters) was then transferred to the tube containing the CLP sample. The tube was then placed on a flat bed shaker with shaking for two, four or six hours. After shaking, all excess fluid was removed from the tube (e.g., no visible fluid in the tube). Last, the tube and sample were weighed and recorded as W3. The saline holding capacity (SHC) was calculated by dividing the mass of the fluid absorbed by the mass of the dry crosslinked polyacrylate polymer, for example, SHC (g/g)=(W3−W2)/(W1). According to the present disclosure, cross-linked cation-binding polymers, including polyacrylate CLP particles prepared according to the methods disclosed herein, had a saline holding capacity of 20 g/g, 30 g/g, 40 g/g, or more. Alternatively stated, such cross-linked cation-binding polymers, including where the polyelectrolyte is polyacrylate, can absorb 20-fold, 30-fold, 40-fold, or more of their mass in a saline solution.

### Example 9

The saline holding capacity of a cross-linked polyelectrolyte polymer, such as a cross-linked polyacrylate polymer, may be determined by known methods in the art.

In an exemplary method, a saline absorption capacity for salts of cross-linked cation-binding polyelectrolyte, for example cross-linked polyacrylate salts of CLP is determined by first rinsing the polymer with hydrochloric acid to convert the salt form to the acid form. The saline absorption capacity of the acid form is then determined.

For example the saline absorption capacity of CaCLP particles can be determined as follows. Phosphate equilibration buffer of 50 mM phosphate, 154 mM NaCl was prepared by dissolving 19.5 g trisodium phosphate dodecahydrate (Na₃PO₄.12H₂O, molecular weight 380.12) in approximately 950 mL of deionized water with pH adjustment to 7.0 with 1N HCl and then diluting to a final volume of 1000 mL with deionized water. A disposable polypropylene chromatography tube was weighed and then 0.1 g of CaCLP CLP particles were transferred to the tube and reweighed. 10 mL 1N HCl was added to the tube containing the sample and the tube was placed on a flat bed shaker and shaken for 30 minutes. The fluid was drained from the tube by gravity. Another 10 mL of 1N HCl was transferred into the tube and shaken for another 30 minutes. The fluid was drained from the tube by gravity. Another 10 mL of 1N HCl was transferred to the tube and shaken for 60 minutes. The fluid was drained from the tube by gravity. 10 mL of deionized water was transferred to the tube and the water immediately suctioned off using a vacuum flask and vacuum pump or a faucet-mounted water aspirator, until there was no visible fluid in the tube. This water rinse step was repeated two more times. 10 milliliters of the pH 7.0 phosphate buffer was transferred to the tube and shaken for 15 minutes. Using a vacuum flask and vacuum pump, or a faucet-mounted water aspirator, the fluid that was not absorbed into the CLP particles was suctioned off so that there was no visible fluid in the tube. 10 mL of the pH 7.0 phosphate buffer was transferred to the tube and the polymer was permitted to swell for 30 minutes then fluid suctioned off until there was no visible fluid in the tube. This was repeated twice with 15 minute swell times and then with a 3 hour swell time so that the total swelling time was four hours. After suctioning off the fluid the tube with swollen polymer was weighed.

The amount of phosphate buffered saline absorbed by the polymer was determined by subtracting the original weight of the tube with dry polymer from the weight of the tube with swollen polymer.

The saline absorption capacity was determined by dividing the amount of saline absorbed by the polymer by the weight of the polymer used in the test (g/g CLP).

### Example 10

This example describes the effects of H—CLP, e.g., prepared as described in Examples 1 and 3, and CaCLP (prepared by addition of calcium counterions to CLP during manufacture, e.g., as described in Examples 5 and 7) on fecal and urinary ion excretion and fecal mass in rats.

In an exemplary study, Ca—CLP with calcium added as counterions during manufacturing at levels as described in Table 5 were prepared using the methods described in Example 5 and 7 including drying in a vacuum oven. Each of these CaCLP polymers were tested in groups of six rats to determine the effect of the percent calcium on the CaCLP on the fecal excretion of Na and K and on the mass of feces excreted.

TABLE 5
CLP Forms Manufactured

| Group | CLP Form |
|---|---|
| 1 | None (control) |
| 2 | H-CLP |
| 3 | 6.9% CaCLP |
| 4 | 10.5% CaCLP |
| 5 | 34.1% CaCLP |
| 6 | 31.1% CaCLP |
| 7 | 41.6% CaCLP |
| 8 | 62% CaCLP |
| 9 | 71% CaCLP |
| 10 | 76% CaCLP |

Ca—CLP was prepared as described in Examples 5 and 7 with drying in a vacuum oven. The mixture was stirred and left to react overnight at room temperature. The mixture was then placed into a vacuum oven and heated at approximately 60° C.

Ca—CLP or H—CLP was mixed at a level of 5% into pulverized LabDiet 5012 and the mixture was processed through a food blender several times until the food/CLP powder was uniform in color and size. Daily measurements of rat weight and 24-hour food intake, water intake, urine output, and fecal output were recorded. Dosing started on Day 1. On Days 4, 5 and 6 24-hour feces and urine were collected for ICP-AES analysis of fecal Na, fecal K and urine P. Samples were digested for ICP-AES analysis by placing the sample in flask, adding an aqueous solution of 5% trace metal grade concentrated nitric acid, and heating to boiling. 30% hydrogen peroxide was then added in small aliquots until the solution was clear and vigorous foaming from addition of hydrogen peroxide had ceased. The digested samples were analyzed by ICP/AES (inductively coupled plasma atomic emission spectroscopy) for fecal sodium, fecal potassium, and urinary phosphate. Changes in fecal sodium and potassium excretion levels from control (rats on rat chow and no polymer) were calculated (i.e., control fecal sodium was subtracted from fecal sodium in the treatment groups).

As shown in Table 6, fecal sodium excretion was relatively independent of the percentage of calcium counterions on the CLP until a decrease between 34-42%. With and an approximately linear decrease from 42 to 71% while Fecal K decreased approximately linearly between 0-40% Ca—CLP and then remained relatively constant.

The effect of different amounts of calcium counterion on Ca—CLP on daily urinary phosphorus (P) excretion and fecal mass are also shown in Table 6 as change from control ((i.e., control urinary phosphorus excretion levels were subtracted from urinary phosphorus levels from treatment groups).

Urinary phosphorus declined in an approximately linearly with was percent calcium counterion on Ca—CLP. Urinary excretion of phosphorus is a measure of the acid/base status of the rat with increasing urine phosphorus correlating with a shift to a more acidic state.

The effect of the percent calcium counterion on Ca—CLP on fecal weight was determined and the change from control daily fecal weights are shown in Table 8 (i.e., control fecal weight was subtracted from fecal weights in treatment groups). Fecal weight was approximately constant between 0 and 31% Ca—CLP. Between 31 and 42% CaCLP the fecal weight dropped significantly and then became approximately constant to 71% CaCLP.

TABLE 6
Change From Control in Daily Fecal Sodium, Fecal
Potassium, Urinary Phosphorous, and Fecal Weight
in Rats Administered H-CLP or Ca-CLP

| | Δ Fecal Na | Δ Fecal K | Δ Urinary | Δ Fecal Fluid |
|---|---|---|---|---|
| | Excretion | Excretion | P Excretion | Excretion |
| CLP Form | (mg/d) | (mg/d) | (mg/d) | (g/day) |
| H-CLP | 30.8 | 84.2 | 29.5 | 6.0 |
| 6.9% CaCLP | 28.9 | 78.7 | 22.6 | 5.9 |
| 10.5% CaCLP | 41.0 | 62.3 | 28.8 | 6.3 |
| 31.1% CaCLP | 40.9 | 29.3 | 12.1 | 6.0 |
| 34.1% CaCLP | 32.7 | 16.6 | 8.9 | 3.5 |
| 41.6% CaCLP | 19.6 | 6.8 | 4.0 | 1.4 |
| 62% CaCLP | 13.6 | 5.8 | 5.6 | 1.6 |
| 71% CaCLP | 13.2 | 8.9 | 1.2 | 1.8 |

### Example 11

This example describes the effects of H—CLP (e.g., prepared as described in Examples 1 and 3) and CaCLP (e.g., prepared by addition of calcium counterions to CLP during manufacturing as described in Examples 5 and 7) with and without added CaCO₃ on fecal and urinary ion excretion and fecal mass in rats.

In this exemplary study, the effect of Ca—CLP or H—CLP with or without calcium carbonate base (administered as TUMS®) on the fecal excretion of Na and K, fecal mass, and urinary excretion of P was studied in rats comparing CLP neutralization by calcium added during manufacturing (6.9% CaCLP or 25% CaCLP) and by CaCO₃ mixed into the feed as Tums (0 to 0.75 equivalents). 6.9% CaCLP and 25% CaCLP were manufactured using the method of Example 9 with vacuum drying of the CLP particles.

Multiple groups of 6 rats were fed diets containing CLP (H—CLP, 6.9% Ca—CLP, or 25% CaCLP) mixed with TUMS® (0 to 0.75 equivalents of calcium carbonate) as 5 w/w % of their daily diet. Each group received a different treatment as described in Table 5. The diet was prepared by mixing Ca—CLP or H—CLP, TUMS® where required, and pulverized LabDiet 5012 and then processing the mixture with a food blender several times until the powder was uniform in color and size.

Daily measurements of rat weight, food intake, water intake, urine output, and fecal output were recorded. Dosing started on Day 1. On days 4, 5, and 6 24-hour feces and urine were collected for ICP-AES analysis of fecal Na, fecal K and urine P. Samples were digested for ICP by placing each sample into a flask, adding an aqueous solution of 5% trace metal grade concentrated nitric acid, and heating to boiling. 30% hydrogen peroxide was then added in small aliquots until the solutions were clear and the vigorous foaming after additions of hydrogen peroxide had ceased. The digested samples were analyzed by ICP for fecal sodium, fecal potassium, and urinary phosphate. Changes in excretion over control (rats on rat chow and no polymer) fecal sodium, and potassium excretion levels were calculated and are shown in Table 7 below (i.e., control fecal sodium and potassium excretion levels were subtracted from fecal sodium and potassium levels from treatment groups).

As shown in Table 7, Administration of CLP with and without base increased fecal excretion of both sodium and potassium compared to control for all formulations tested. Increasing the degree of CLP neutralization had no significant effect on fecal sodium excretion whereas fecal potassium excretion decreased approximately linearly with increases in neutralization. Neutralization of CLP by addition of calcium counterions at 6.9% and 25% during manufacture and/or by addition of CaCO3 had similar effect on fecal sodium and potassium excretion and on urinary P excretion.

As shown in Table 7, co-administration of HCLP or CaCLP with base decreased urinary phosphorous levels. Urinary excretion of phosphorus is a measure of the acid/base status of the rat with increasing urine phosphorus correlating with a shift to a more acidic state. When H—CLP was administered without base high urinary phosphorous values were observed. When H—CLP or Ca—CLP was administered with increasing amounts of CaCO₃ base the urinary excretion of phosphorus was decreased and in the range of 65 to 72% base the urinary phosphorus was not different from controls. Neutralization of CLP by addition of calcium counterions at 6.9% and 25% during manufacture and/or by addition of CaCO3 had similar effect on urinary P excretion.

TABLE 7
Effect of Added CaCO₃ on Daily Fecal Sodium and
Potassium Excretion and Urinary Phosphorous Excretion
in Rats Administered HCLP or CaCLP

| | Base | Total CLP | | | |
|---|---|---|---|---|---|
| | added | Carboxyl | | | |
| | as CaCO3 | Neutrali- | Fecal Na | Fecal K | Urinary P |
| CLP | (Equiva- | zation¹ | Excretion | Excretion | Excretion |
| Form | lent Ratio) | (%) | (mg/d) | (mg/d) | (mg/d) |
| H-CLP | 0 | 0 | 27.5 | 83.0 | 27.7 |
| H-CLP | 0.5 | 50 | 26.9 | 35.3 | 8.5 |
| H-CLP | .75 | 75 | 27.6 | 23.9 | −1.9 |
| H-CLP | .62 | 62 | 27.5 | 35.8 | 4.5 |
| 6.9% CaCLP | 0 | 6.9 | 28.9 | 78.7 | 22.6 |
| 6.9% CaCLP | .65 | 71.9 | 31.1 | 18.8 | −2.8 |
| 25% CaCLP | 0 | 25 | 22.7 | 53.3 | 13.9 |
| 25% CaCLP | .4 | 65 | 23.2 | 33.9 | 0.9 |
| 25% CaCLP | .50 | 75 | 20.8 | 22.3 | −5.3 |

¹Total carboxyl neutralization is the sum of the percent of Ca counterions added during manufacturing and the equivalents of base mixed into the feed.

The addition of CaCO₃ to the rat feed decreases the fecal excretion of sodium and potassium. At an equivalent total CLP neutralization ratio fecal sodium and potassium excretion and urinary P excretion were similar regardless of whether the neutralization was from calcium base added during manufacture or calcium base added as CaCO₃ (Tums®).

A comparison of the data in Examples 10 and 11 show that for low levels of total neutralization (0 to about 30-35%), base added as CaCO₃ or base added as counterions during manufacture had similar effects on fecal sodium and potassium excretion. At higher levels of calcium counterions added during manufacture (from approximately 30-35% CaCLP to 71% CaCLP), fecal excretion of sodium and potassium decreased to a greater extent than for the same total neutralization of CLP obtained with HCLP, 6.9% CaCLP or 25% CaCLP with added CaCO₃. The effect of neutralization of urinary phosphorus excretion did not depend whether the calcium counterions were added during manufacture or base was mixed into the formulation as CaCO₃.

Changes in fecal weight compared to control are shown in Table 8 (control fecal weight was subtracted from fecal weight in the treatment groups).

TABLE 8
Effect of Added CaCO₃ on Daily Fecal Excretion
in Rats Administered 0-25% CaCLP

| | Base added | Total CLP | |
|---|---|---|---|
| | as CaCO3 | Carboxyl | Δ Fecal Fluid |
| | (Equivalent | Neutralization | Excretion |
| CLP Form | Ratio) | Potential (%)¹ | (g/day) |
| H-CLP | 0 | 0 | 6.8 |
| H-CLP | 0.5 | 50 | 4.5 |
| H-CLP | .75 | 75 | 3.7 |
| H-CLP | .62 | 62 | 3.8 |
| 6.9% CaCLP | 0 | 6.9 | 6.4 |
| 6.9% CaCLP | .65 | 71.9 | 3.3 |
| 25% CaCLP | 0 | 25 | 5.8 |
| 25% CaCLP | .4 | 65 | 3.0 |
| 25% CaCLP | .50 | 75 | 1.7 |

¹Total carboxyl neutralization is the sum of the percent of Ca counterions added during manufacturing and the equivalents of base mixed into the feed.

A comparison of the data in Examples 10 and 11 is provided in Table 9 for ease of comparison. At low levels of reacted calcium (0 to about 30-35%) on the polymer, base added as CaCO3 or calcium counterions added during manufacturing had similar effects on fecal sodium and potassium excretion. At higher levels of calcium counterions added during manufacturing (about 30-35% to 75%) fecal excretion of sodium and potassium were decreased compared to mixtures of CaCO₃ with low levels of reacted Ca+CaCO₃ or H—CLP. Base added as CaCO3 or calcium counterions added during manufacturing had similar effects on urinary P excretion.

TABLE 9
Comparison of Fecal Na and K Excretion from Tables
8 and 9 for for Total CLP Carboxyl Neutralization
Between 31 and 75% Neutralization

| | Base added | Total CLP | | |
|---|---|---|---|---|
| | as CaCO3 | Carboxyl | Fecal Na | Fecal K |
| | (Equivalent | Neutralization¹ | Excretion | Excretion |
| CLP Form | Ratio) | (%) | (mg/d) | (mg/d) |
| 31.1% CaCLP | 0 | 31.1 | 32.8 | 16.6 |
| 34.1% CaCLP | 0 | 34.1 | 40.9 | 29.3 |
| 41.6% CaCLP | 0 | 41.6 | 19.6 | 6.8 |
| H-CLP | 0.5 | 50 | 26.9 | 35.3 |
| 62% CaCLP | 0 | 62 | 13.6 | 5.8 |
| H-CLP | 0.62 | 62.5 | 27.5 | 35.8 |
| 25% CaCLP | 0.4 | 65 | 23.2 | 33.9 |
| 71% CaCLP | 0 | 71 | 13.2 | 8.9 |
| 6.9% CaCLP | 0.65 | 71.9 | 31.1 | 18.8 |
| H-CLP | 0.75 | 75 | 27.6 | 23.9 |
| 25% CaCLP | 0.5 | 75 | 20.8 | 22.3 |

¹Total carboxyl neutralization is the sum of the percent of Ca counterions added during manufacturing and the equivalents of base mixed into the feed.

### Example 12

An open-label, multiple-dose escalation clinical trial was performed in twenty-five healthy human subjects divided into five groups (Table 10). One control group received no treatment, one group received 7.5 g H—CLP/day with meals, one group received 15 g H—CLP/day with meals, one received 15 g H—CLP/day one hour before meals, and one group received 25 g H—CLP/day with meals. Subjects remained in the clinical research unit for the duration of the study.

H—CLP was prepared according to Examples 1 and 3. The H—CLP polymer was milled to break up the bead structure and reduce the particle size. The milled H—CLP was then filled into capsules with 0.7 g per capsule.

The objectives of the clinical trial included (1) determination of the safety, tolerability and efficacy of H—CLP to remove, i.e., altered fecal excretion of, sodium, calcium, magnesium, potassium, iron, copper, zinc and/or phosphorous; (2) to determine whether administration of H—CLP altered the amount of fluid absorbed, i.e., altered fecal weight, per gram of H—CLP administered; (3) to determine whether administration of H—CLP altered measures of acidosis, including serum total bicarbonate, urine pH, and urine phosphorous; and (4) to determine whether administration of H—CLP altered serum potassium levels. For all outcomes, treated groups were compared to the control group.

The primary endpoints included net sodium balance compared among treated and control groups. Secondary endpoints included change in stool weight compared among treated and control groups; net balance of calcium, magnesium, potassium, iron, copper, zinc and phosphorous compared among treated and control groups; fluid consumed and excreted in the treated groups compared with the control group; and safety and tolerability based upon review of vital signs, clinical safety labs and adverse events.

H—CLP was administered with water, 4 times a day for a total of 9 days (a total of 36 consecutive doses). For each dose group of five subjects, H—CLP was administered one hour before or just after each of 4 standardized meals or snacks as shown in Table 10. Doses were given at the scheduled time (+/−10 minutes) for each subject.

TABLE 10
Dose Groups and Feeding Status at Dose Administration

| | Number of | Dose H-CLP | Timing of | Duration of |
|---|---|---|---|---|
| Group | Subjects | (g/day) | Dosing | Dosing (d) |
| Control | 5 | 0 | | 9 |
| A | 5 | 7.5 | Just after each | 9 |
| | | | meal or snack | |
| B | 5 | 15 | Just after each | 9 |
| | | | meal or snack | |
| C | 5 | 15 | One hour before | 9 |
| | | | each meal or | |
| | | | snack | |
| D | 5 | 25 | Just after each | 9 |
| | | | meal or snack | |

Diet was controlled with all participants having identical meals. Each day all meals and snacks representing one subject were homogenized and the sodium, potassium, calcium, phosphorus, iron, copper, zinc and magnesium content determined. All meals provided to the subjects were controlled for the number of calories, level of sodium (5000 mg per day +/−100 mg), fiber content (10-15 g per day), fat content and approximate recommended Dietary Reference Intakes. Subjects were requested to consume all of their meals. Meals that were not fully consumed were collected for an entire twenty-four hour period, weighed and frozen for possible metal analysis.

Subjects fasted for at least eight hours at screening and four hours at admission prior to the collection of blood and urine samples for clinical laboratory tests. Fasting was not required prior to urine and blood, samples taken during the study. Water ad libitum was allowed during the periods of fasting.

Stool weight, fecal electrolytes and fluid balance were determined daily. Serum samples were collected daily and the concentration of sodium, potassium, magnesium, calcium, phosphorus and carbon dioxide determined. All urine specimens were collected and volume recorded. An aliquot of a daily afternoon urine sample was analyzed for pH and osmolality. Urine samples were pooled for each 24-hour period and an aliquot sampled for sodium, potassium, calcium, phosphorous and magnesium analysis.

All feces eliminated after consumption of the first controlled meal were collected as individual samples in tared collection containers. The color and consistency of the stool were noted, the sample weighed, then frozen and stored at or below −20° C. All fecal collections were analyzed for sodium, potassium, magnesium, calcium, phosphorous, iron, zinc and copper content. Fecal weights for all samples eliminated in each 24-hour period were added together to determine the total fecal weight per subject per day.

Daily fecal and urine weight, urine osmolality and pH, and daily fecal and urine content and concentrations of sodium, calcium, magnesium, potassium and phosphorus (plus copper, iron and zinc only in the stool) were determined for each subject and each treatment group. Daily fluid balance (fluid intake-output) and daily net balance of sodium, magnesium, calcium, potassium and phosphorus were calculated based on the analysis of diet, urine and stool samples for each patient and each group.

Daily parameters were compared for each H—CLP dose group and the control group. A steady state effect of dosing with H—CLP administered 4 times daily was reached after 4 days of dosing. Daily parameters were also averaged for days 5-9 for each group and treatment groups compared to the control group.

Fecal metal excretion (e.g., sodium, potassium, magnesium and calcium) for doses of H—CLP between 0 and 25 g are shown in Tables 11 to 14 below. Daily excretion of sodium, potassium, magnesium and calcium for the control group are shown in Table 11. The average daily value of metal cation excretion on days 1 to 9 for the treatment groups are compared to the average value for the control group and are shown for 7.5 g of H—CLP daily (Group A, Table 12), for 15 g of H—CLP daily taken immediately after meal (Group B, Table 13), and for 25 g of H—CLP daily (Group D, Table 14). Fasting before administration of H—CLP did not significantly affect ion excretion.

TABLE 11
Fecal Metal Excretion (mg/day)-0 grams H-CLP (Control Group)

| | Sodium | Potassium | Magnesium | Calcium |
|---|---|---|---|---|
| | Excretion | Excretion | Excretion | Excretion |
| Day | (mg/day) | (mg/day) | (mg/day) | (mg/day) |
| 1 | 33.5 | 280.1 | 141.2 | 554.9 |
| 2 | 70.5 | 906.5 | 342.1 | 1663.4 |
| 3 | 12.1 | 239.6 | 112.1 | 691.2 |
| 4 | 114.8 | 728.7 | 292.6 | 2005.6 |
| 5 | 21.5 | 394.4 | 149.1 | 1134.1 |
| 6 | 32.8 | 453.3 | 182.2 | 1351.7 |
| 7 | 151.5 | 680.2 | 289.2 | 2003.1 |
| 8 | 44.9 | 289.4 | 120.2 | 1059.0 |
| 9 | 45.5 | 259.0 | 109.0 | 866.0 |

TABLE 12
Changes in Fecal Metal Excretion Over Control (mg/day) for
Subjects Administered 7.5 grams of H-CLP Daily (Group A)

| | Δ Sodium | Δ Potassium | Δ Magnesium | Δ Calcium |
|---|---|---|---|---|
| | Excretion | Excretion | Excretion | Excretion |
| Day | (mg/day) | (mg/day) | (mg/day) | (mg/day) |
| 1 | 22.5 | 313.6 | 130.3 | 742.7 |
| 2 | 62.7 | 147.1 | −17.5 | 147.2 |
| 3 | 348.6 | 1188.1 | 127.1 | 758.0 |
| 4 | 473.0 | 1554.0 | −17.7 | −130.4 |
| 5 | 362.1 | 981.7 | 2.2 | −71.2 |
| 6 | 365.3 | 1182.3 | 27.3 | 105.2 |
| 7 | 531.6 | 1223.3 | −22.4 | −445.6 |
| 8 | 524.5 | 1763.4 | 159.6 | 728.3 |
| 9 | 298.0 | 1104.9 | 72.6 | 247.9 |

TABLE 13
Changes in Fecal Metal Excretion Over Control (mg/day) for
Subjects Administered 15 grams of H-CLP Daily (Group B)

| | Δ Sodium | Δ Potassium | Δ Magnesium | Δ Calcium |
|---|---|---|---|---|
| | Excretion | Excretion | Excretion | Excretion |
| Day | (mg/day) | (mg/day) | (mg/day) | (mg/day) |
| 1 | −16.2 | 254.2 | 78.2 | 390.3 |
| 2 | 70.4 | 222.2 | −102.2 | −541.3 |
| 3 | 338.5 | 1442.6 | 66.9 | 240.5 |
| 4 | 565.9 | 1195.0 | −96.9 | −829.6 |
| 5 | 1032.2 | 2531.8 | 78.3 | 167.6 |
| 6 | 1158.3 | 1744.8 | 49.9 | −29.0 |
| 7 | 1003.5 | 1422.0 | −26.5 | −519.2 |
| 8 | 1103.0 | 1555.7 | 103.5 | 342.3 |
| 9 | 808.2 | 1888.7 | 108.3 | 350.8 |

TABLE 14
Changes in Fecal Metal Excretion Over Control (mg/day) for
Subjects Administered 25 grams of H-CLP Daily (Group D)

| | Δ Sodium | Δ Potassium | Δ Magnesium | Δ Calcium |
|---|---|---|---|---|
| | Excretion | Excretion | Excretion | Excretion |
| Day | (mg/day) | (mg/day) | (mg/day) | (mg/day) |
| 1 | 86.9 | 302.9 | 80.3 | 470.6 |
| 2 | 779.8 | 347.7 | −142.0 | −693.1 |
| 3 | 723.5 | 1314.9 | 13.6 | 46.8 |
| 4 | 1947.1 | 2956.3 | −38.3 | −593.6 |
| 5 | 1763.2 | 3644.0 | 43.7 | −63.5 |
| 6 | 1905.8 | 4872.7 | 130.0 | 617.3 |
| 7 | 2489.5 | 4631.2 | 34.0 | −248.4 |
| 8 | 2529.0 | 3631.2 | 191.9 | 598.6 |
| 9 | 1641.6 | 2248.8 | 84.5 | 189.6 |

For each treatment group the amount of Na and K excreted in the feces increased between days 1 to 4 and then became fairly constant on days 5 to 9. The net change from the control group in the average daily fecal sodium and potassium content for days 5-9 was determined for each treatment group and shown in Table 15.

TABLE 15
Change in Daily Average of Fecal Sodium and Potassium Excretion
and Serum Potassium Compared to Control for Days 5-9

| Dose | Dose | Na | K | Serum K |
|---|---|---|---|---|
| (g) | Administration | (mg/day) | (mg/day) | (mmol/L) |
| 7.5 | With meals | 417 | 1228 | −0.5 |
| 15 | With meals | 981 | 1825 | −0.8 |
| 15 | One hour prior | 1034 | 1749 | −0.9 |
| | to meals | | | |
| 25 | With meals | 2046 | 3668 | −1.5 |

The administration of HCLP results in a dose dependent increase in the fecal excretion of sodium and potassium.

Serum potassium levels were also evaluated daily. The change in average serum potassium for the treatment groups from the average for the control group on Days 5 to 9 values are shown in Table 15. Serum potassium decreased from control values in all treatment groups.

Measures of acidosis included total serum CO2 and urine phosphate. The average change from control in these parameters for Days 5-9 are shown in Table 16.

TABLE 16
Average Change from Control in Acid/Base
Parameters for Days 5-9

| | | | Total serum | Urine | Fecal |
|---|---|---|---|---|---|
| Dose | Time of | Urine | CO2 | Phosphate | Phosphate |
| (g) | Administration | pH | (mmol/L) | (mg/day) | (mg/day) |
| 7.5 | With meal | −1.3 | −2.3 | 255 | −181 |
| 15 | With meal | −1.21 | −4.4 | 341 | −365 |
| (fed) | | | | | |
| 15 | One hour prior | −0.78 | −4.4 | 389 | −363 |
| (fasted) | to meal | | | | |
| 25 | With meal | −0.79 | −8.8 | 341 | −305 |

For all doses of HCLP there was an apparent acidosis as measured by these parameters. The decrease from control in total serum CO2 and serum phosphate were dose dependent.

Administration of HCLP led to an increase in fecal weight in a dose dependent manner as shown in Table 17. This increase in fecal weight was not associated with diarrhea but is expected to be due to water entrapped in the superabsorbent polymer.

TABLE 17
Average Change from Control in Fecal Weight for Days 5-9

| Dose | Time of | Fecal Wt |
|---|---|---|
| (g) | Administration | (g) |
| 7.5 | With meal | 121 |
| 15 | With meal | 173 |
| 15 | One hr prior | 162 |
| | to meal | |
| 25 | With meal | 360 |

Administration of HCLP led to a decrease in serum phosphate, a dose dependent increase in fecal excretion of sodium and potassium and a dose dependent increase in fecal weight.

Administration of HCLP also caused acidosis.

### Example 13

An open-label clinical trial was performed in twelve healthy human subjects. Each patient received an equivalent of 15 g H—CLP/day as either 25% CaCLP (n=6) or 60% CaCLP (n=6), divided into three doses, administered one hour prior to meals. Subjects remained in the clinical research unit for the duration of the study.

25% CaCLP and 60% CaCLP were prepared according to Example 5. After cation exchange to load the polyacrylate with calcium, the polymer was milled to break up the bead structure and reduce the particle size. The CaCLP powder was mixed into pudding immediately prior to dosing. The subjects were required to eat the entire pudding aliquot.

The clinical trial evaluated whether administration of CaCLP when compared to a baseline period (1) altered fecal excretion of sodium, potassium, or phosphorous (2) altered measures of acidosis including serum total bicarbonate, urine pH and urine phosphorus, (3) altered serum potassium levels and (4) altered fecal weight.

After a 5 day baseline period, CaCLP was administered in pudding, 3 times a day for a total of 7 days (a total of 14 doses). For 25% CaCLP the dose was 16 g (5.33 g tid). For 60% CaCLP the dose was 18 g (6 g tid). A dose of 16 g of 25% CaCLP and 18 g 60% CaCLP each delivered an equivalent number of moles of cation exchange carboxyl groups as 15 g of H—CLP (208 mEq).

Diet was controlled with all participants having identical meals. Subjects were requested to consume all of their meals.

Subjects fasted for at least eight hours at screening and four hours at admission prior to the collection of blood and urine samples for clinical laboratory tests. Fasting was not required prior to collection of urine and blood samples taken during the study. Water ad libitum was allowed during the periods of fasting.

Twenty four hour daily stool and urine samples were collected daily and evaluated for stool weight, fecal electrolytes, urine pH, and urine phosphorus. Daily serum samples were evaluated for serum potassium and total bicarbonate. Fecal samples were evaluated by ICP for the concentration of sodium, potassium, calcium and magnesium. All urine specimens were collected and volume recorded. Urine samples were pooled for each 24-hour period and an aliquot sampled for sodium, potassium, calcium, phosphorous and magnesium analysis.

Daily parameters for the treatment period were compared to baseline, with daily parameters for days 3-6 averaged and compared to the average for treatment days 10-13. The average change from baseline in fecal excretion of sodium and potassium are shown in Table 18. An increase in percent calcium counterion from 25 to 60% resulted in a decrease in fecal sodium and potassium excretion and a larger drop in serum potassium.

TABLE 18
Average change from baseline in fecal sodium,
fecal potassium and serum potassium

| Ca | | | |
|---|---|---|---|
| Counterion | Fecal Na | Fecal K | Serum K |
| (%) | (mg/d) | (mg/d) | (mmol/L) |
| 25 | 1005 | 2129 | −0.15 |
| 25% CaCLP | | | |
| 60 | 169 | 1103 | −0.43 |
| 60% CaCLP | | | |

Measures of acidosis included urine pH, total serum CO2, and urine phosphate. The average change from baseline in these parameters for Days 7-13 are shown in Table 20. The change from baseline in serum bicarbonate and urine pH were similar for 25% CaCLP and 60% CaCLP. The urinary phosphorus excretion decreased by a factor of 10 with an increase from 25% to 60% calcium counterion. This was paralleled by a decrease in fecal phosphorus excretion by a factor of 10 between 25% and 60% calcium counterion.

TABLE 19
Average Change from Baseline in
Acidosis Parameters for Days 7-13

| | | | Urinary | Fecal |
|---|---|---|---|---|
| Ca | Total serum | | Phosphorus | Phosphorus |
| Counterion | CO2 | Urine | Excretion | Excretion |
| (%) | (mmol/) | pH | (mg/day) | (mg/day) |
| 25 | −0.9 | −0.48 | 277 | −223 |
| 25% CaCLP | | | | |
| 60 | −0.7 | −0.63 | 27 | −19 |
| 60% CaCLP | | | | |

Administration of CaCLP led to an increase in fecal weight as shown in Table 18. This increase in fecal weight was not associated with diarrhea but is expected to be due to water entrapped in the superabsorbent polymer. An increase in the percent of calcium counterion from 25 to 60% decreased the fecal weight.

TABLE 20
Average Change from Baseline in Fecal Weight for Days 7-13

| Ca | |
|---|---|
| Counterion | Fecal Wt |
| (%) | (g) |
| 25 | 197 |
| 25% CaCLP | |
| 60 | 87 |
| 60% CaCLP | |

### Example 14

An open-label clinical trial was performed in twenty four healthy human subjects. Each patient received an equivalent of 15 g H—CLP/day as either 25% CaCLP or 50% CaCLP, divided into three doses, administered one hour prior to meals. Subjects remained in the clinical research unit for the duration of the study.

TABLE 21
Dose Groups

| | Number of | CLP | Dose | Dose of | Dose of |
|---|---|---|---|---|---|
| Group | Subjects | formulation¹ | Administration | CLP (g)² | CaCO₃ (g) |
| 1 | 6 | H-CLP + 75% CaCO₃ | Capsules with | 15 | 0 |
| | | | water | | |
| 2 | 6 | 25% CaCLP + | Powder mixed | 16 | 5.2 |
| | | 50% CaCO₃ | in pudding | | |
| 3 | 6 | 25% CaCLP + | Capsules with | 16 | 3.6 |
| | | 35% CaCO₃ | water | | |
| 4 | 6 | 50% CaCLP | Powder mixed | 17 | 0 |
| | | | in pudding | | |

¹25% CaCLP is H-CLP with 25% of the carboxyl groups reacted with calcium base; 50% CaCO3 is CaCO3 added to the formulation at a mass that will neutralize 50% of the carboxyl groups in an equivalent dose of H-CLP.
²All CLP doses gave an equivalent dose of carboxyl groups as 15 g of H-CLP

25% CaCLP and 50% CaCLP were prepared according to Example 5. After cation exchange to load the polyacrylate with calcium, the polymer was milled to break up the bead structure and reduce the particle size. The CaCLP powder was mixed into pudding immediately prior to dosing. The subjects were required to eat the entire pudding aliquot.

The clinical trial evaluated whether administration of CLP when compared to a baseline period (1) altered fecal excretion of sodium, potassium or phosphorus (2) altered measures of acidosis including serum total bicarbonate, urine pH and urine phosphorus, (3) altered serum potassium levels, and (4) altered fecal weight.

After a 5 day baseline period, CLP was administered in capsules with water or in pudding, twice a day (before breakfast and before dinner) for a total of 7 days (a total of 14 doses) as shown in Table 21. Groups 1 and 3 had the CLP formulation administered in capsules with water and Groups 2 and 4 had the CLP formulation mixed into pudding immediately prior to administration. All groups were administered an equivalent number of moles of cation exchange carboxyl groups as 15 g of H—CLP (208 mEq).

Diet was controlled with all participants having identical meals. Subjects were requested to consume all of their meals.

Subjects fasted for at least eight hours at screening and four hours at admission prior to the collection of blood and urine samples for clinical laboratory tests. Fasting was not required prior to urine and blood samples taken during the study. Water ad libitum was allowed during the periods of fasting.

Twenty four hour daily stool and urine samples were collected daily and evaluated for stool weight, fecal electrolytes, urine pH, and urine phosphorus. Daily serum samples were evaluated for serum potassium and total bicarbonate. Fecal samples were evaluated by ICP for the concentration of sodium, potassium, calcium, and magnesium. All urine specimens were collected and volume recorded. Urine samples were pooled for each 24-hour period and an aliquot sampled for sodium, potassium, calcium, phosphorous, and magnesium analysis.

Daily parameters for the treatment period were compared to baseline, with daily parameters for days 3-6 averaged and compared to the average for treatment days 10-13. The average change from baseline in fecal excretion of sodium and potassium are shown in Table 22. All groups had an increase in fecal sodium and potassium excretion compared to baseline.

TABLE 22
Average change from baseline in fecal sodium,
fecal potassium and serum potassium

| | | Fecal | | |
|---|---|---|---|---|
| | CLP | Na | Fecal K | Serum K |
| Group | formulation^{1,2} | (mg/d) | (mg/d) | (mmol/L) |
| 1 | H-CLP + 75% CaCO3 | 534 | 1258 | −0.2 |
| 2 | 25% CaCLP + | 342 | 1415 | −0.2 |
| | 50% CaCO3 | | | |
| 3 | 25% CaCLP + | 671 | 1907 | 0.0 |
| | 35% CaCO3 | | | |
| 4 | 50% CaCLP | 419 | 1518 | −0.1 |

¹25% CaCLP is H-CLP with 25% of the carboxyl groups reacted with calcium base; 50% CaCO3 is CaCO3 added to the formulation at a mass that will neutralize 50% of the carboxyl groups in an equivalent dose of H-CLP.
²All CLP doses gave an equivalent dose of carboxyl groups as 15 g of H-CLP

Measures of acidosis included urine pH, total serum CO2 and urine phosphate. The average change from baseline in these parameters for Days 7-13 are shown in Table 23.

TABLE 23
Average Change from Baseline in Acidosis Parameters for Days 7-13

| | | | | Urinary | Fecal |
|---|---|---|---|---|---|
| | | Total serum | | Phosphorus | Phosphorus |
| | CLP | CO2 | Urine | Excretion | Excretion |
| Group | formulation^{1,2} | (mmol/) | pH | (mg/day) | (mg/day) |
| 1 | H-CLP + 75% CaCO3 | −0.3 | −0.3 | 110 | −101 |
| 2 | 25% CaCLP + | 0.7 | −0.2 | 148 | −160 |
| | 50% CaCO3 | | | | |
| 3 | 25% CaCLP + | −0.9 | −0.9 | 115 | −127 |
| | 35% CaCO3 | | | | |
| 4 | 50% CaCLP | −1.0 | −0.4 | 150 | −81 |

¹25% CaCLP is H-CLP with 25% of the carboxyl groups reacted with calcium base; 50% CaCO3 is CaCO3 added to the formulation at a mass that will neutralize 50% of the carboxyl groups in an equivalent dose of H-CLP.
²All CLP doses gave an equivalent dose of carboxyl groups as 15 g of H-CLP

Administration of CLP led to an increase in fecal weight as shown in Table 24. This increase in fecal weight was not associated with diarrhea but is expected to be due to water entrapped in the superabsorbent polymer.

TABLE 24
Average Change from Baseline in Fecal Weight for Days 7-13

| | CLP | Fecal Wt |
|---|---|---|
| Group | formulation^{1,2} | (g) |
| 1 | H-CLP + 75% CaCO3 | 159 |
| 2 | 25% CaCLP + | 95 |
| | 50% CaCO3 | |
| 3 | 25% CaCLP + | 197 |
| | 35% CaCO3 | |
| 4 | 50% CaCLP | 102 |

¹25% CaCLP is H-CLP with 25% of the carboxyl groups reacted with calcium base; 50% CaCO3 is CaCO3 added to the formulation at a mass that will neutralize 50% of the carboxyl groups in an equivalent dose of H-CLP.
²All CLP doses gave an equivalent dose of carboxyl groups as 15 g of H-CLP

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary only, with the true scope and spirit of the disclosure being indicated by the following claims. All references, patents, and patent applications referred to in this application are herein incorporated by reference in their entireties.

What is claimed is:

1. A crosslinked cation-binding polymer comprising:
a. monomers comprising carboxylate groups; and
b. calcium and/or magnesium cations,
wherein the calcium and/or magnesium cations are counterions to about 15% to about 35% of the carboxylate groups in said polymer, and
wherein the polymer comprises no more than about 5% sodium cations as counterions to the carboxylate groups in said polymer.

2. The polymer of claim 1, wherein said calcium and/or magnesium cations are counterions to about 20% to about 30% of the carboxylate groups in said polymer.

3. The polymer of claim 1, wherein said calcium and/or magnesium cations are counterions to about 21% of the carboxylate groups in said polymer.

4. The polymer of claim 1, wherein said calcium and/or magnesium cations are counterions to about 22% of the carboxylate groups in said polymer.

5. The polymer of claim 1, wherein said calcium and/or magnesium cations are counterions to about 25% of the carboxylate groups in said polymer.

6. The polymer of claim 1, wherein said calcium and/or magnesium cations are counterions to about 29% of the carboxylate groups in said polymer.

7. The polymer of claim 1, wherein said calcium and/or magnesium cations consist of calcium cations.

8. The polymer of claim 1, wherein counterions that are not calcium, magnesium, or sodium are hydrogen.

9. The polymer of claim 1, wherein the counterions to said carboxylate groups consist of said calcium and/or magnesium counterions, and hydrogen counterions.

10. The polymer of claim 1, wherein said monomer is acrylic acid, an acrylic acid derivative, or a salt thereof.

11. The polymer of claim 10, wherein said monomer is acrylic acid or a salt thereof.

12. The polymer of claim 1, wherein said polymer is crosslinked with a crosslinker selected from diethelyeneglycol diacrylate (diacryl glycerol), triallylamine, tetraallyloxyethane, allylmethacrylate, 1,1,1-trimethylolpropane triacrylate (TMPTA), and divinylbenzene.

13. The polymer of claim 12, wherein said crosslinked polymer is derived from acrylic acid monomers and TMPTA.

14. The polymer of claim 1, wherein said polymer has an in vitro saline absorption capacity of at least about 20 times its weight.

15.-16. (canceled)

17. A composition comprising:
a. the polymer of claim 1; and
b. a base,
wherein said base is present in an amount sufficient to provide up to about 0.8 equivalents of base per equivalent of carboxylic acid groups in said polymer.

18.-29. (canceled)

30. A composition comprising:
a. a crosslinked polyacrylate polymer comprising acrylic acid repeat units that comprise carboxylic acid groups, and calcium and/or magnesium cations, wherein said calcium cations are counterions to about 15% to about 35% of the carboxylate groups in said polymer, and wherein the polymer comprises no more than about 5% sodium cations as counterions to the carboxylate groups in said polymer; and
b. calcium carbonate,
wherein said calcium carbonate is present in an amount sufficient to provide up to about 0.8 equivalents of base per equivalent of carboxylic acid groups in said polymer.

31.-46. (canceled)

47. A dosage form comprising:
a. the polymer of claim 1; and
b. a base,
wherein said base is present in an amount sufficient to provide up to about 0.8 equivalents of base per equivalent of carboxylic acid groups in said polymer.

48.-171. (canceled)

172. A method of treating a disease or disorder in a subject in need thereof, said method comprising administering to said subject the polymer of claim 1.

173. A method of treating a disease or disorder in a subject in need thereof, said method comprising:
a. identifying a disease or a disorder in said subject, or identifying a risk that said subject will develop a disease or disorder; and
b. administering to said subject an effective amount of the polymer of claim 1.

174. The method of claim 172, wherein said disease or disorder is one or more of: heart failure, a renal insufficiency disease, end stage renal disease, liver cirrhosis, chronic renal insufficiency, chronic kidney disease, fluid overload, fluid maldistribution, edema, pulmonary edema, peripheral edema, angioneurotic edema, lymphedema, nephrotic edema, idiopathic edema, ascites, cirrhotic ascites, chronic diarrhea, excessive interdialytic weight gain, high blood pressure, hyperkalemia, hypernatremia, abnormally high total body sodium, hypercalcemia, tumor lysis syndrome, head trauma, an adrenal disease, Addison's disease, salt-wasting congenital adrenal hyperplasia, hyporeninemic hypoaldosteronism, hypertension, salt-sensitive hypertension, refractory hypertension, hyperparathyroidism, renal tubular disease, rhabdomyolysis, electrical burns, thermal burns, crush injuries, renal failure, acute tubular necrosis, insulin insufficiency, hyperkalemic periodic paralysis, hemolysis, malignant hyperthermia, pulmonary edema secondary to cardiogenic pathophysiology, pulmonary edema with non-cardiogenic origin, drowning, acute glomerulonephritis, aspiration inhalation, neurogenic pulmonary edema, allergic pulmonary edema, high altitude sickness, Adult Respiratory Distress Syndrome, traumatic edema, cardiogenic edema, allergic edema, urticarial edema, acute hemorrhagic edema, papilledema, heatstroke edema, facial edema, eyelid edema, angioedema, cerebral edema, scleral edema, nephritis, nephrosis, nephrotic syndrome, glomerulonephritis, renal vein thrombosis, and/or premenstrual syndrome.

175.-177. (canceled)
